(19) United States
(12) Patent Application Publication
(10) Pub. No.: US 2004/0087571 A1
(43) Pub. Date: May 6, 2004

(54) Methods of using CCR1 antagonists as immunomodulatory agents

(75) Inventors: Matthew F. Brown, Stonington, CT (US); Anderson S. Gaweco, Stonington, CT (US); Ronald P. Gladue, Stonington, CT (US); John C. Kath, Waterford, CT (US); Christopher S. Poss, Baltic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/687,015

(22) Filed: Oct. 16, 2003

Related U.S. Application Data
(60) Provisional application No. 60/422,579, filed on Oct. 30, 2002.

(51) Int. Cl. A61K 31/397; A61K 31/53; A61K 31/50; A61K 31/4965; A61K 31/505

(52) U.S. Cl. 514/210.17; 514/246; 514/247; 514/255.06; 514/256; 514/354; 514/423

(57) ABSTRACT

The present invention relates to methods of using CCR1 antagonists as immunomodulatory agents. In particular, the present invention relates to methods of using heteroaryl-hexanoic acid amide derivatives of the formula (I)

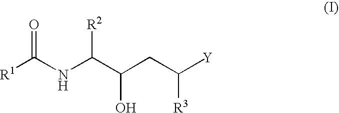

wherein R¹, R², R³, and Y are as described in the specification.

### PRIORITY CLAIM

The present application claims priority to U.S. Patent Application Serial No. 60/422,579, filed Oct. 30, 2002, which is incorporated herein in its entirety.

### BACKGROUND OF THE INVENTION

The present invention relates to methods of using CCR1 antagonists as immunomodulatory agents, in particular methods of using heteroaryl-hexanoic acid amide derivatives.

Compounds of heteroaryl-hexanoic acid amides and their methods of manufacture are disclosed in commonly assigned U.S. Pat. No. 6,403,587B1, filed Feb. 5, 1998, U.S. patent application Ser. No. 09/403,218, filed Jan. 18, 1999, U.S. patent application Ser. No. 09/774,871, filed Feb. 4, 2000, PCT Publication No. WO98/38167, PCT Publication No. WO99/40061, and PCT Publication No. WO01/57023, all of which are incorporated herein by reference in their entireties for all purposes.

### SUMMARY OF THE INVENTION

One aspect of the present invention relates to methods of treating or preventing a disorder or condition selected from the group consisting of fibrosis, Alzheimer's disease, conditions associated with leptin production, sequelae associated with cancer, cancer metastasis, diseases or conditions related to production of cytokines at inflammatory sites, and tissue damage caused by inflammation induced by infectious agents; wherein the method comprises administering to a mammal in need of such treatment or prevention a pharmaceutically effective amount of the compound of formula (I)

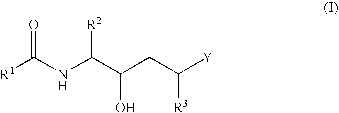

wherein R¹ is (C₂-C₉)heteroaryl optionally substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

R² is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein m is zero, one, two, three or four; wherein each of said phenyl, naphthyl, (C₃-C₁₀)cycloalkyl and (C₂-C₉)heteroaryl moieties of said phenyl-(CH₂)ₘ—, naphthyl-(CH₂)ₘ—, (C₃-C₁₀)cycloalkyl-(CH₂)ₘ— and (C₂-C₉)heteroaryl-(CH₂)ₘ— groups may optionally be substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N(C═O)—, H₂N(C═O)—(C₁-C₆)alkyl, (C₁-C₆)alkyl-HN(C═O)—(C₁-C₆)alkyl, [(C₁-C₆)alkyl]₂N(C═O)—(C₁-C₆)alkyl, H(O═C)—NH—, (C₁-C₆)alkyl(C═O)—NH, (C₁-C₆)alkyl(C═O)[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, or (C₂-C₉)heteroaryl;

R³ is hydrogen, (C₁-C₁₀)alkyl, (C₃-C₁₀)cycloalkyl-(CH₂)ₙ—, (C₂-C₉)heterocycloalkyl-(CH₂)ₙ—, (C₂-C₉)heteroaryl-(CH₂)ₙ— or aryl-(CH₂)ₙ—; wherein n is zero, one, two, three, four, five or six;

wherein the (C₁-C₁₀)alkyl moiety of said R³ (C₁-C₁₀)alkyl group may optionally be substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and wherein any of the carbon-carbon single bonds of said (C₁-C₁₀)alkyl may optionally be replaced by a carbon-carbon double bond;

wherein the (C₃-C₁₀)cycloalkyl moiety of said R³ (C₃-C₁₀)cycloalkyl-(CH₂)ₙ— group may optionally be substituted by one to three substitutents, wherein each substituent is independently hydrogen, halo, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

wherein the (C₂-C₉)heterocycloalkyl moiety of said R³ (C₂-C₉)heterocycloalkyl(CH₂)ₙ— group comprises nitrogen, sulfur, oxygen, >S(═O), >SO₂ or >NR⁶, wherein said (C₂-C₉)heterocycloalkyl moiety of said (C₂-C₉)heterocycloalkyl-(CH₂)ₙ— group may optionally be substituted on any of the ring carbon atoms capable of forming an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

wherein the (C₂-C₉)heteroaryl moiety of said R³ (C₂-C₉)heteroaryl-(CH₂)ₙ— group comprises nitrogen, sulfur or oxygen wherein said (C₂-C₉)heteroaryl moiety of said (C₂-C₉)heteroaryl-(CH₂)ₙ— group may optionally be substituted on any of the ring carbon atoms capable of forming an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl)₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and

wherein said aryl moiety of said R³ aryl-(CH₂)ₙ— group is optionally substituted phenyl or naphthyl, wherein said phenyl and naphthyl may optionally be substituted with from one to three substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O(C₁-C₆)alkyl, H(O═C)—, H(O═C)—(C₁-C₆)alkyl, (C₁-C₆)alkyl(O═C)—, (C₁-C₆)alkyl(O═C)(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

or R³ and the carbon to which it is attached form a five to seven membered carbocyclic ring, wherein any of the carbon atoms of said five membered carbocyclic ring may optionally be substituted with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O(C₁-C₆)alkyl, H(O═C)—, H(O═C)—(C₁-C₆)alkyl, (C₁-C₆)alkyl(O═C)—, (C₁-C₆)alkyl(O═C)(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; wherein one of the carbon-carbon bonds of said five to seven membered carbocyclic ring may optionally be fused to an optionally substituted phenyl ring, wherein said phenyl substitutents may be hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—N H—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

Y is (C₂-C₉)heteroaryl, (C₂-C₉)heterocycloalkyl, R⁵(R)⁶N-sulfonyl or a group of the formula

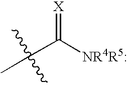

X is O, S, or NR¹²;

R⁴ is hydrogen, (C₁-C₆)alkyl, hydroxy, (C₁-C₆)alkoxy, hydroxy(C₁-C₆)alkyl, (C₁-C₆)alkoxy(C═O)—, (C₃-C₁₀)cycloalkyl-(CH₂)ₚ—, (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ—, wherein p is zero, one, two, three or four; wherein said (C₂-C₉)heterocycloalkyl, (C₂-C₉)heteroaryl, phenyl and naphthyl groups of said (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ— may be optionally substituted on any of the ring atoms capable of supporting an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C), (C₁-C₆)alkyl(O═C)—(C₁-C₆)alkyl, NO₂, amino, (C₁-C₆)alkylamino, [(C₁-C₆)alkyl]2 amino, amino(C₁-C₆)alkyl, (C₁-C₆)alkylamino (C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

or R⁴ and R⁵ together with the nitrogen atom to which they are attached form a (C₂-C₉)heterocycloalkyl group wherein any of the ring atoms of said (C₂Cg)heterocycloalkyl group may optionally be substituted with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C), (C₁-C₆)alkyl(O═C)—(C₁-C₆)alkyl, NO₂, amino, (C₁-C₆)alkylamino, [(C₁-C₆)alkyl]₂ amino, amino(C₁-C₆)alkyl, (C₁-C₆)alkylamino (C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—N H, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

R⁵ is hydrogen, (C₁-C₆)alkyl or amino;

R⁶ is hydrogen, (C₁-C₆)alkyl, (C₁-C₆)alkoxy-(CH₂)_{g}—, (C₁-C₆)alkoxy(C═O)(CH₂)_{g}—, (C₁-C₆)alkyl-(SO₂)—(CH₂)_{g}—, (C₆-C₁₀)aryloxy-(CH₂)_{g}—, (C₆-C₁₀)aryloxy(C═O)(CH₂)_{g}—, or (C₆-C₁₀)aryl-(SO₂)—(CH₂)_{g}—, wherein g is an integer from zero to four; and

R¹² is hydrogen, CN, (C═O)-(C₁-C₉)alkyl, or (SO₂)—(C₁-Cg)alkyl;

with the proviso that when either R⁴ or R⁵ is hydrogen, and the other of R⁴ or R⁵ is (C₁-C₆)alkyl, R² is (C₃-C₁₀)cycloalkyl or isopropyl and R³ is (C₃-C₅)alkyl, phenyl, methylvinyl, dimethylvinyl, halovinyl, hydroxy(C₁-C₃)alkyl or amino(C₁-C₄)alkyl then R¹ must be other than indol-5-yl, 6-azaindol-2-yl, 2,3-dichloro-pyrol-5-yl, 4-hydroxyquinolin-3-yl, 2-hydroxyquinoxalin-3-yl, 6-azaindolin-3-yl, or optionally substituted indol-2 or 3-yl;

or a pharmaceutically acceptable form thereof.

In one preferred embodiment, the compound of formula I has the formula Ia

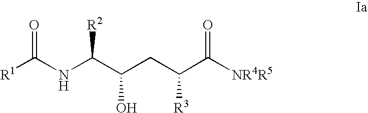

wherein R¹, R², R³, R⁴ and R⁵ are as described above.

In another preferred embodiment, R¹ is optionally substituted pyrazolo[3,4b]pyridinyl, cinnolinyl, pyridinyl, 6,7-dihydro-5H-[1]pyrindinyl, benzothiazolyl, indolyl, pyrazinyl, benzoimidazolyl, benzofuranyl, benzo[b]thiophenyl, naphthalenyl, quinoxalinyl, isoquinolinyl, 5,6,7,8-tetrahydro-quinolin-3-yl or quinolinyl. More preferably, R¹ is optionally substituted pyrazolo[3,4-b]pyridin-5-yl, cinnolin-4-yl, pyridin-2-yl, 6,7-dihydro-5H-[1]pyrindin-3-yl, benzothiazol-2-yl, indol-2-yl, pyrazin-2-yl, benzoimidazol-2-yl, benzofuran-2-yl, benzo[b]thiophen-2-yl, naphthalen-2-yl, quinoxalin-2-yl, quinoxalin-6-yl, isoquinolin-1-yl, isoquinolin-3-yl, isoquinolin-4-yl, 5,6,7,8-tetrahydro-quinolin-3-yl, quinolin-2-yl, quinolin-3-yl, quinolin-4-yl or quinolin-6-yl. More preferably, R¹ is optionally substituted quinoxalin-2-yl, quinoxalin-6-yl, quinolin-2-yl, quinolin-3-yl, quinolin-4-yl or quinolin-6-yl.

In another preferred embodiment, R² is optionally substituted benzyl.

Still another preferred embodiment includes compounds wherein R³ is optionally substituted (C₁-C₁₀)alkyl or (C₃-C₁₀)cycloalkyl-(CH₂)ₙ—, more preferably, R³ is optionally substituted n-butyl, t-butyl, isobutyl, n-pentyl, 2-methyl-pentyl, cyclopentyl, or cyclohexyl, more preferably, R³ is substituted by fluoro or hydroxy, more preferably, R³ is 4,4-difluoro-cyclohexylmethyl, 2-fluoro-2-methyl-butyl, isobutyl, or 1-hydroxy-cyclohexyl.

In another preferred embodiment, the compound is:

quinoxaline-2-carboxylic acid 4(R)-carbamoyl-1 (S)-(3-chloro-benzyl)-2(S),7-dihydroxy-7-methyl-octyl]-amide;

7,8-difluoro-quinoline-3-carboxylic acid (1S)-benzyl-4(R)-carbamoyl-2(S),7-dihydroxy-7-methyl-octyl)-amide;

6,7,8-trifluoro-quinoline-3-carboxylic acid (1(S)-benzyl-4(R)-carbamoyl-2(S),7-dihydroxy-7-methyl-octyl)-amide;

quinoxaline-2-carboxylic acid [4(R)-carbamoyl-1 (S)-(3-fluoro-benzyl)-2(S),7-dihydroxy-7-methyl-octyl]-amide;

quinoxaline-2-carboxylic acid (1 (S)-benzyl-2(S),7-dihydroxy-4(R)-hydroxycarbamoyl-7-methyl-octyl)-amide;

quinoxaline-2-carboxylic acid [4(R)-carbamoyl-1 (S)-(2-chloro-benzyl)-2(S),7-dihydroxy-7-methyl-octyl]-amide;

quinoxaline-2-carboxylic acid [1 (S)-(2-fluoro-benzyl)-2(S),7-dihydroxy-4(R)-hydroxycarbamoyl-7-methyl-octyl]-amide;

quinoxaline-2-carboxylic acid [4(R)-carbamoyl-1 (S)-(2-fluoro-benzyl)-2(S),7-dihydroxy-7-methyl-octyl]-amide;

quinoxaline-2-carboxylic acid [1 (S)-(3,4-difluoro-benzyl)-2(S),7-dihydroxy-4(R)-hydroxycarbamoyl-7-methyl-octyl]-amide;

quinoxaline-2-carboxylic acid [4(R)-carbamoyl-1 (S)-(3,4-difluoro-benzyl)₂(S),7-dihydroxy-7-methyl-octyl]-amide; or

quinoxaline-2-carboxylic acid (4(R)-carbamoyl-2(S),7-dihydroxy-7-methyl-1 (S)-naphthalen-1-ylmethyl-octyl)-amide.

In a further preferred embodiment, the method comprises administering a pharmaceutically effective amount of a composition comprising the compound of formula I or Ia and a pharmaceutically acceptable carrier.

Another preferred embodiment includes the methods described above wherein the disorder or condition is selected from the group consisting of pulmonary fibrosis, fibrosis associated with end-stage renal disease, fibrosis caused by radiation, tubulointerstitial fibrosis, subepithelial fibrosis, scleroderma, hepatic fibrosis, primary and secondary biliary cirrhosis, obesity, cachexia, anorexia, type II diabetes, hyperlipidemia and hypergonadism, sequelae associated with multiple myeloma, breast cancer, joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith, viral induced encephalomyelitis or demyelination, gastrointestinal inflammation, bacterial meningitis, cytomegalovirus, adenoviruses, Herpes viruses, fungal meningitis, lyme disease, and malaria.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

In this specification and in the claims that follow, reference will be made to a number of terms that shall be defined to have the following meanings:

Unless otherwise indicated, “alkyl” groups referred to herein, as well as the alkyl moieties of other groups referred to herein (e.g., alkoxy), may be linear or branched, saturated (e.g. alkanes) or unsaturated (e.g. alkenes and alkynes) and they may also be cyclic (e.g., cyclopropyl, cyclobutyl, cyclopentyl, cyclohexyl or cycloheptyl) or be linear or branched and contain cyclic moieties. Such alkyl and alkoxy groups may be optionally substituted with one, two or three halogen and/or hydroxy atoms, preferably fluorine atoms.

Unless otherwise indicated, “halogen,” “halide,” and “halo” includes fluorine, chlorine, bromine, and iodine.

“(C₃-C₁₀)cycloalkyl” when used herein refers to cycloalkyl groups containing zero, one or two levels of unsaturation such as cyclopropyl, cyclobutyl, cyclopentyl, cyclopentenyl, cyclohexyl, cyclohexenyl, 1,3-cyclohexadiene, cycloheptyl, cycloheptenyl, bicyclo[3.2.1]octane, norbornanyl, and the like.

“(C₂-C₉)heterocycloalkyl” when used herein refers to pyrrolidinyl, tetrahydrofuranyl, dihydrofuranyl, tetrahydropyranyl, pyranyl, thiopyranyl, aziridinyl, oxiranyl, methylenedioxyl, chromenyl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, chromanyl, and the like. One of ordinary skill in the art will understand that the connection of said (C₂-C₉)heterocycloalkyl rings is through a carbon or a sp³ hybridized nitrogen heteroatom.

“(C₂-C₉)heteroaryl” when used herein refers to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl, benzoxazinyl, and the like. One of ordinary skill in the art will understand that the connection of said (C₂-C₉)heterocycloalkyl rings is through a carbon atom or a sp³ hybridized nitrogen heteroatom.

“Aryl” when used herein refers to phenyl or naphthyl.

The symbol “-” when used between two groups of a substituent shall mean a chemical bond.

By “pharmaceutically acceptable” is meant a material that is not biologically or otherwise undesirable, i.e., the material may be administered to an individual along with the selected compound without causing any substantially undesirable biological effects or interacting in a deleterious manner with any of the other components of the pharmaceutical composition in which it is contained.

“Pharmaceutically acceptable forms” when used herein refers to any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs.

The term “subject” is meant an individual. Preferably, the subject is a mammal such as a primate, and more preferably, a human. Thus, the “subject” can include domesticated animals, livestock, and Iaboratory animals.

In general, “effective amount” or “effective dose” means the amount needed to achieve the desired result or results (treating or preventing the disorder or condition). One of ordinary skill in the art will recognize that the potency and, therefore, an “effective amount” can vary for the various compounds used in the invention. One skilled in the art can readily assess the potency of the compounds.

Unless otherwise noted, numerical values described and claimed herein are approximate. Variation within the values may be attributed to equipment calibration, equipment errors, purity of the materials, among other factors. Additionally, variation may be possible, while still obtaining the same result.

Compounds of the formulas I and Ia may be prepared using any suitable method. Furthermore, the reaction Schemes 1-10 described herein for the compounds of formula I and Ia may also be used. Unless otherwise indicated, the substituents of all structural formulas in the reaction schemes and discussion that follow are the same as that defined above.

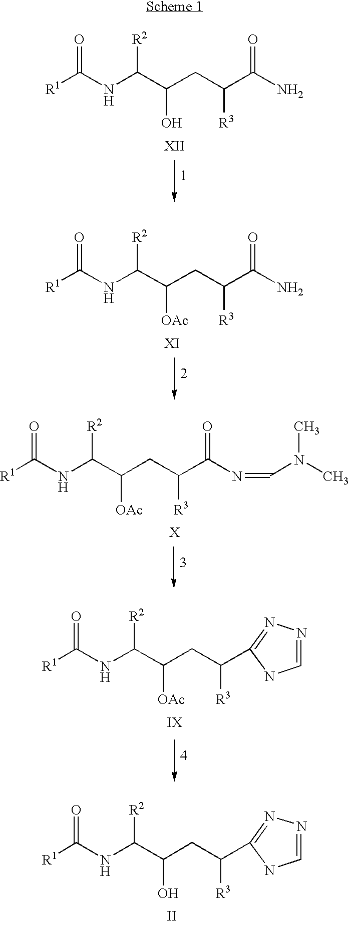

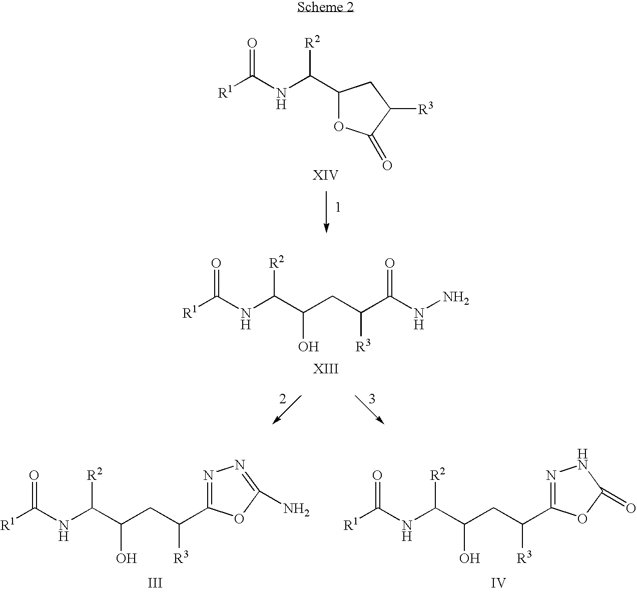

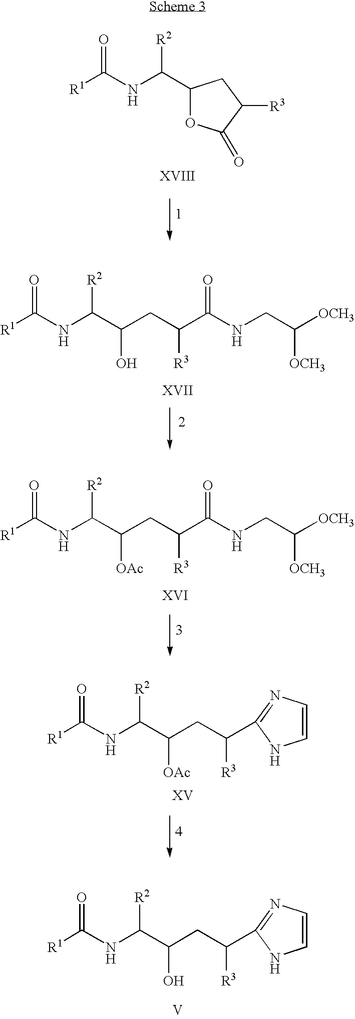

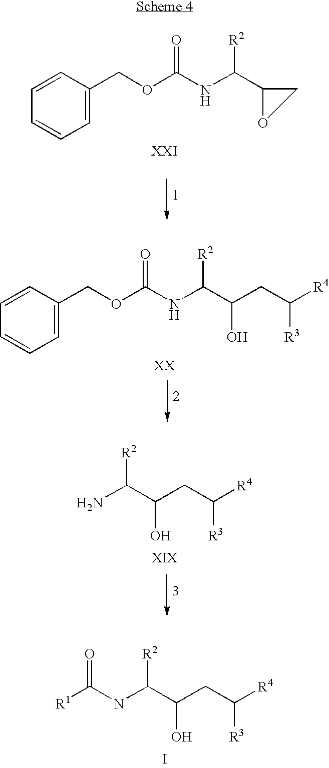

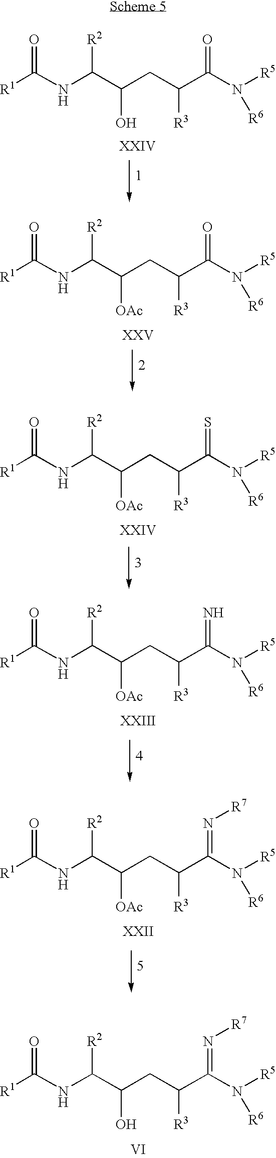

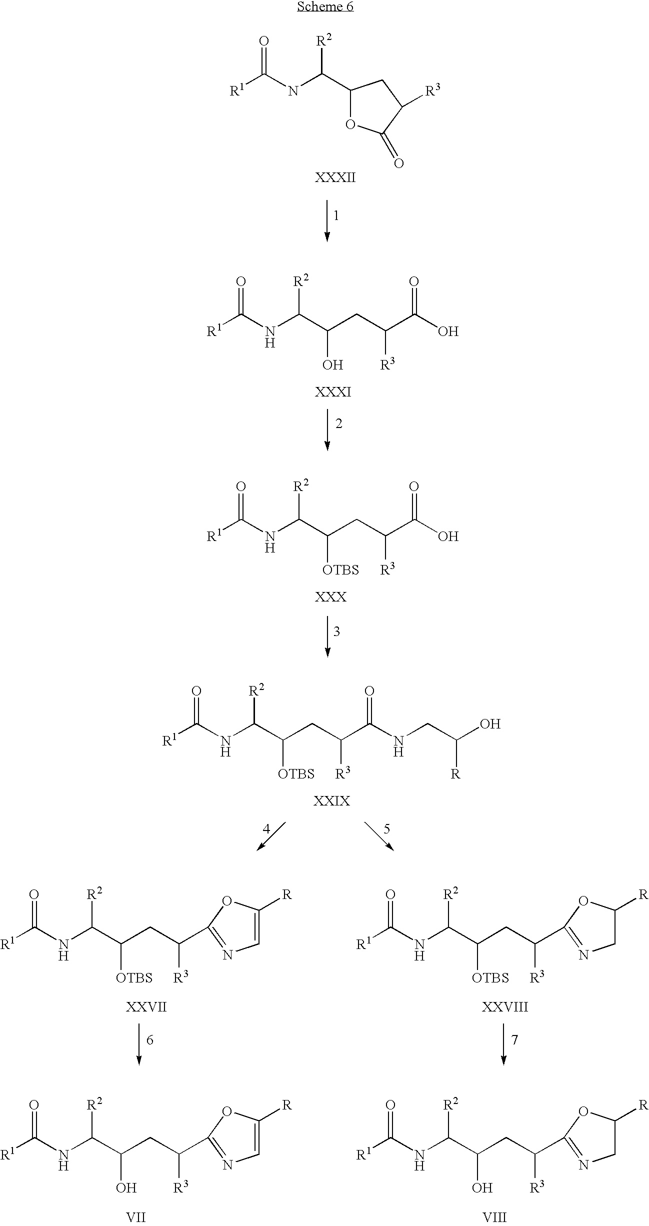

In reaction 1, of Scheme 1, the alcohol compound of formula XII is converted to the corresponding acetate compound of formula XI by reacting XII with acetic anhydride in the presence of 4-dimethylaminopyridine (DMAP) and pyridine. The reaction 1 stirred at a temperature between about 0° C. to about room temperature, preferably about 0° C., for a time period between about 1 hour to about 3 hours, preferably about 2 hours.

In reaction 2 of Scheme 1, the compound of formula XI is converted to the corresponding compound of formula X by reacting XI with N,N-dimethylformamide dimethyl acetal in the presence of a polar protic solvent, such as methanol. The reaction is stirred at a temperature between about 40° C. to about 60° C., preferably about 50° C., for a time period between about 30 minutes to about 2 hours, preferably about 1 hour.

In reaction 3 of Scheme 1, the compound of formula X is converted to the corresponding triazole compound of formula IX by reacting X with hydrazine in the presence of acetic acid. The reaction is stirred at a temperature between about 40° C. to about 60° C., preferably about 50° C., for a time period between about 30 minutes to about 2 hours, preferably about 1 hour.

In reaction 4 of Scheme 1, the compound of formula IX is converted to the corresponding compound of formula II by deprotecting 1× with potassium carbonate in the presence of methanol at room temperature overnight.

In reaction 1 of Scheme 2, the lactone compound of formula XIV is converted to the corresponding hydrazide compound of formula XII by reacting XIV with hydrazine in a polar protic solvent, such as methanol. The reaction is stirred at room temperature overnight.

In reaction 2 of Scheme 2, the hydrazine compound of formula XIII is converted to the corresponding 1,2,4-oxadiazole compound of formula III by reacting XII with cyanogen bromide in the presence of dioxane and water. The reaction is heated to reflux for a time period between about 30 minutes to about 2 hours, preferably about 1 hour.

In reaction 3 of Scheme 2, the hydrazide compound of formula XII is converted to the corresponding compound of formula IV by reacting XII with CDI in the presence of a base, such as triethylamine, and a polar aprotic solvent, such as tetrahydrofuran. The reaction is stirred at room temperature for a time period between about 10 hours to about 20 hours, preferably overnight.

In reaction 1 of Scheme 3, the lactone compound of formula XVIII is converted to the corresponding compound of formula XVII by reacting XVIII with aminoacetaldehyde dimethyl acetal in the presence of dioxane. The reaction is stirred overnight at a temperature between about 30° C. to about 70° C., preferably about 50° C.

In reaction 2 of Scheme 3, the alcohol compound of formula XVII is converted to the corresponding acetate compound of formula XVI according to the procedure described above in reaction 1 of Scheme 1.

In reaction 3 of Scheme 3, the compound of formula XVI is converted to the corresponding imidazole compound of formula XV by reacting XVI with ammonium acetate in the presence of acetic acid. The reaction is stirred at a temperature between about 105° C. to about 125° C., preferably about 115° C., for a time period between about 3 hours to about 5 hours, preferably about 4 hours.

In reaction 4 of Scheme 3, the compound of formula XV is converted to the corresponding compound of formula V according to the procedure described above in reaction 4 of Scheme 1.

In reaction 1 of Scheme 4, the epoxide compound of formula XXI is converted to the corresponding compound of formula XX by reacting XXI with a compound of the formula, CHR³(R)⁴, in the presence of a base, such as n-butyllithium, and a polar aprotic solvent, such as tetrahydrofuran. The reaction is carried out at a temperature between about −78° C. to about 0° C., preferably about −78° C., for a time period between about 1 hours to about 4 hours, preferably about 2 hours.

In reaction 2 of Scheme 4, the compound of formula XX is converted to the corresponding compound of formula XIX by removal of the carbobenzyloxy protecting group through hydrogenation of XX in the presence of palladium on carbon and a polar protic solvent, such as ethanol. The reaction is carried out at a temperature between about 0° C. to room temperature, preferably room temperature, for a time period between about 1 hour to about 24 hours, preferably about 15 hours.

In reaction 3 of Scheme 4, the compound of formula XIX is converted to the corresponding compound of formula I by reacting XIX with a compound of the formula, R¹—CO—Cl, in the presence of a base, such as triethylamine, and a polar aprotic solvent, such as methylene chloride. The reaction is carried out at a temperature between about −20° C. to about 40° C., preferably about 0° C., for a time period between about 1 hour to about 24 hours, preferably about 2 hours.

In reaction 1 of Scheme 5, the compound of formula XXVI is converted to the corresponding compound of formula XXV according to the procedure described above in reaction 1 of Scheme 1.

In reaction 2 of Scheme 5, the amide compound of formula XXV is converted to the thioacetamide compound of formula XXIV by reacting XXV with Lawesson's Reagent, [2,4-bis(4-methoxyphenyl)-1,3-dithia-2,4-diphosphetane-2,4-disulfide], in the presence of a polar aprotic solvent, such as tetrahydrofuran. The reaction is carried out at a temperature between about 0° C. to about 60° C., preferably about 25° C., for a time period between about 1 hour to about 24 hours, preferably about 5 hours.

In reaction 3 of Scheme 5, the thioacetamide compound of formula XXIV is converted to the corresponding compound of formula XXIII by first treating XXIV with methyl iodide, followed by reacting the compound so formed with ammonia in methyl alcohol. The reaction is carried out at a temperature between about 0° C. to about 60° C., preferably about 25° C., for a time period between about 1 hour to about 24 hours, preferably about 15 hours.

In reaction 4 of Scheme 5, the compound of formula XXIII is converted to the corresponding compound of formula XXII by reacting XXII with (a) R⁸ sulfonyl chloride when R⁷ is R⁸S(O)₂; (b) cyanogen bromide when R⁷ is cyano; (c) L-N═C═O when R⁷ is an amide and L is a leaving group; or (d) an acyl chloride compound of the formula, R⁸—CO—Cl, when R⁷ is R⁸C(O).

In reaction 5 of Scheme 5, the compound of formula XXII is converted to the corresponding compound of formula VI according to the procedure described above in reaction 1 of Scheme 1. In reaction 1 of Scheme 6, the lactone of formula XXXII is converted to the corresponding compound of formula XXXI by reacting XXXII with a base, such as lithium hydroxide, in the presence of a mixture of water and a polar aprotic solvent, such as tetrahydrofuran. The reaction is carried out at a temperature between about 0° C. to about 60° C., preferably about 25° C., for a time period between about 1 hour to about 24 hours, preferably about 2 hours.

In reaction 2 of Scheme 6, the compound of formula XXXI is converted to the corresponding compound of formula XXX by reacting XXXI with tert-butyldimethylsilyl chloride in the presence of imidazole and polar protic solvent, such as dimethylformamide. The reaction is carried out at a temperature between about 0° C. to about 60° C., preferably about 25° C., for a time period between about 1 day to 7 days, preferably 1 day.

In reaction 3 of Scheme 6, the compound of formula XXX is converted to the corresponding compound of formula XXIX by reacting XXX with a compound of the formula

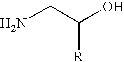

in the presence of 1-hydroxybenzotriazole hydrate, 1-(3-dimethylaminopropyl)-3-ethylcarbodiimide and a polar aprotic solvent, such as methylene chloride. The reaction is carried out at a temperature between about 0° C. to about 30° C., 10 preferably about 25° C., for a time period between about 1 hour to about 24 hours, preferably about 25 hours.

In reaction 4 of Scheme 6, the compound of formula XXIX is converted to the corresponding oxazole compound of the formula XXVII by first oxidizing XXIX with the Dess-Martin periodinane oxidation reagent of the formula

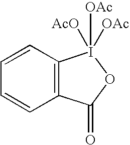

followed by treating the compound so formed with triphenylphosphine, triethylamine, hexachloroethane and a polar aprotic solvent, such as methylene chloride. The reaction is carried out at a temperature between about 0° C. to about 40° C., preferably about 25° C., for a time period between about 5 hours to about 24 hours, preferably about 15 hours.

In reaction 5 of Scheme 6, the compound of formula XXIX is converted to the corresponding oxazoline compound of formula XXVIII by treating XXIX with triphenylphosphine, hexachloroethane, triethylamine and a polar aprotic solvent, such as methylene chloride. The reaction is carried out at a temperature between about 0° C. to about 40° C., preferably about 25° C., for a time period between about 5 hours to about 24 hours, preferably about 15 hours.

In reaction 6 of Scheme 6, the compound of formula XXVII is converted to the corresponding compound of formula VII by treating XXVII with tert-butyl ammonium fluoride. The reaction is carried out at a temperature between about 0° C. to about 40° C., preferably about 25° C., for a time period between about 1 hour to about 24 hours, preferably about 2 hours.

In reaction 7 of Scheme 6, the compound of formula XXVIII is converted to the corresponding compound of formula VII according to the procedure described above in reaction 6 of Scheme 6.

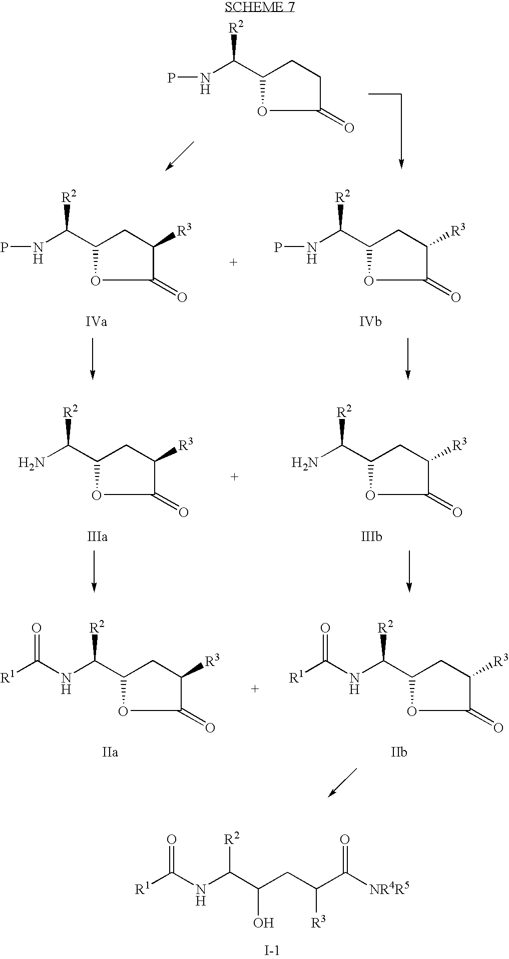

Scheme 7 refers to the preparation of compounds of the formula I having the exact stereochemistry

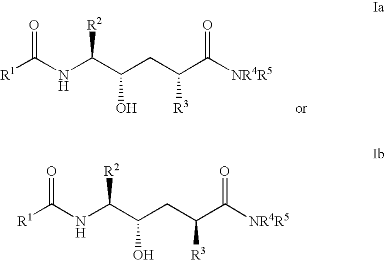

Compounds of the formula Ia and Ib, or any of the intermediates thereof, can be separated by column chromatography according to methods well known to those of ordinary skill in the art, to yield pure compounds of the formula Ia and Ib.

Referring to Scheme 7, compounds of the formula I-1, wherein either or both R⁴ or R⁵ are other than hydrogen, are prepared from compounds of the formula II (i.e. IIa and IIb) by reaction with a compound of the formula R⁴(R)⁵NH in a polar solvent at a temperature from about 0° C. to about 100° C., preferably the boiling point of the solvent used, i.e. 65° C. when methanol is the solvent. Suitable solvents include, alcohols, such as methanol, ethanol, or butanols or ethers such as glyme or dioxane (an acid catalyst is preferably used with an ether solvent). Preferably the solvent is dioxane.

Alternatively, compounds of formula I-1, wherein either or both R⁴ and R⁵ are hydrogen, can be prepared from compounds of formula II, (i.e. IIa and IIb) by reaction with ammonia or another volatile amine in a polar solvent at a temperature from about −10° C. to about 35° C., preferably at about 30° C. Suitable solvents include, alcohols, such as methanol, ethanol, or butanols; or ethers such as glyme or dioxane (an acid catalyst may be used with an ether solvent). Preferably the solvent is methanol.

Compounds of formula II are prepared by coupling a compound of formula III (i.e. IIIa and IIIb) with an acid of the formula RlCO₂H. Such a coupling reaction is generally conducted at a temperature of about −30° C. to about 80° C., preferably about 0° C. to about 25° C. Examples of suitable coupling reagents which activate the carboxylic acid functionality are dicyclohexylcarbodiimide/hydroxybenzotriazole (DCC/HBT), N-3-dimethylaminopropyl-N′-ethylcarbodiimide (EDC)/HBT, 2-ethyoxy-1-ethoxycarbonyl-1,2-dihydroquinoline (EEDQ), carbonyl diimidazole (CDI)/dimethylaminopyridine (DMAP), and diethylphosphorylcyanide. The coupling is conducted in an inert solvent, preferably an aprotic solvent, such as acetonitirile, dichloromethane, chloroform, and dimethylformamide. The preferred solvent is dichloromethane.

For a discussion of other conditions used for amide coupling see HoubenWeyl, Vol. XV, part 11, E. Wunsch, Ed., George Theime Veriag, 1974, Stuttgart, and those described in M. Bodanszky. Principles of Peptide Synthesis, Springer-Verlag, Berlin (1984) and The Peptides, Analysis, Synthesis and Biology (ed. E. Gross and J. Meienhofer), Vois 1-5. (Academic Press, New York) 1979-1983.

The compounds of formula III, wherein R³ is (C₁-C₁₀)alkyl, (C₃-C₁₀)cycloalkyl(CH₂)ₙ, (C₂-C₉)heterocycloalkyl-(CH₂)ₙ—, (C₂-C₉)heteroaryl-(CH₂)ₙ—, or aryl-(CH₂)ₙ— can be prepared by deprotection of compounds of the formula IV (i.e. IVa and IVb). Suitable protecting groups, of the formula P, include carbobenzyloxy, t-butoxy carbonyl or 9-fluorenyl-methylenoxy carbonyl.

For example:

(a) If the protecting group, P, of the compound of the formula IV is carbobenzyloxy, the latter may be removed by hydrogenation with a nobel metal catalyst such as palladium or palladium hydroxide on carbon in the presence of hydrogen. The hydrogenation is generally conducted at a temperature of about 0° C. to about 100° C., preferably about 20° C. to 50° C.

(b) If the protecting group, P, is t-butoxycarbonyl group, such group may be removed by acidolysis. Acidolysis may be conducted with HCl in dioxane or with trifluoracetic acid in methylene chloride at a temperature of about −30° C. to about 70° C., preferably about −50° C. to about 35° C.

(c) If the protecting group, P, is 9-fluorenylmethylenoxycarbonyl, such group may be removed by treatment with an amine base, preferably piperidine. This reaction may be run in piperidine as solvent at 10° C. to about 100° C., preferably at 25° C.

Compounds of the formula II, wherein R³ is substituted (C₁-C₁₀)alkyl, (C₃-C₁₀)cycloalkyl-(CH₂)ₙ— or (C₂-C₉)heterocycloalkyl-(CH₂)ₙ— may be prepared from compounds of the formula IV, wherein R³ is (C₁-C₁₀)alkyl, (C₃-C₁₀)cycloalkyl-(CH₂)ₙ— or (C₂-C₉)heterocycloalkyl-(CH₂)ₙ—, wherein one of the carbon-carbon single bonds is replaced by a carbon-carbon double bond, by methods well known to those of ordinary skill in the art. Specifically, one example of introduction of substitution into the R³ group, a compound of formula II, wherein R³ is (C₁-C₁₀)alkyl substituted by one to three fluoro groups can be prepared from compounds of the formula IV, wherein R³ is (C₁-C₁₀)alkyl, wherein one of the carbon-carbon single bonds of said (C₁-C₁₀)alkyl has been replaced by a carbon-carbon double bond, by reaction with hydrogen fluoride in pyridine (i.e. pyridinium poly(hydrogen fluoride), in a reaction inert solvent. Suitable solvents include cyclohexane, toluene or benzene, preferably benzene. The aforesaid reaction is run at a temperature from about −78° C. to about 35° C. Preferably, this reaction is carried out in benzene at about 25° C.

Compounds of the formula IV, wherein R³ is (C₁-C₁₀)alkyl, (C₃-C₁₀)cycloalkyl(CH₂)ᵣ—, (C₂-C₉)heterocycloalkyl-(CH₂)ₙ—, (C₂-C₉)heteroaryl-(CH₂)ₙ— or aryl-(CH₂)ₙ—, wherein n is other than zero, can be prepared by reaction of a compound of formula V with a compound of the formula R³-L, wherein L is a leaving group, in the presence of a strong base in an aprotic polar solvent. Suitable leaving groups include chloro, fluoro, bromo, iodo, mesylate, triflate or tosylate. Preferably, the leaving group is a triflate, iodide or bromide. Triflates may be easily prepared according to the method of Beard, et al., J. Org Chem., 38, 3673 (1973). Suitable bases include lithium dialkyl amides such as lithium N-isopropyl-N-cyclohexylamide or potassium hydride. Suitable solvents include ethers (such as THF, glyme or dioxane) benzene or toluene, preferably THF. The aforesaid reaction is conducted at about −78° C. to about 0° C., preferably at about −78° C.

Alternatively, compounds of the formula IV, wherein R³ is (C₁-C₁₀)alkyl, (C₃-C₁₀)cycloalkyl-(CH₂)ₙ— or (C₂-C₉)heterocycloalkyl-(CH₂)ₙ— can be prepared by reaction of a compound of formula V with an aldehyde or ketone precursor of R³ in an aldol condensation. For example, a compound of the formula V can be reacted with a compound of the formula R³(═O) in the presence of a base, to form an aldol intermediate of the formula

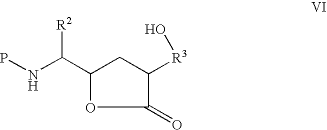

which may be isolated and taken on to final product or converted directly in the same reaction step to a compound of the formula IV by the loss of water. The degree of completion for the conversion of compounds of the formula II to the aldol product of formula I may be assessed using one or more analytical techniques, such as thin layer chromatography (tic) or mass spectrometry. In some instances it may be possible or desirable to isolate the intermediate of formula VI. In such case, the compound of formula VI may be converted into the compound of formula IV by the elimination of water using techniques which are familiar to those skilled in the art, for example, by heating to the reflux temperature a solution of the compound of formula VI in a solvent such as benzene, toluene or xylene, in the presence of a catalytic amount of phosphorous pentoxide, benzene- or p-toluene-sulfonic acid with provision for the removal of the water generated, preferably (methoxycarbonylsulfamoyl)-triethylammonium hydroxide (Burgess reagent). Such water removal techniques may involve the use of molecular sieves or a Dean-Stark trap to isolate the water created as an azeotrope with the solvent.

The aldol reaction is typically carried out in a polar solvent such as DMSO, DMF, tetrahydrofuran (THF), methanol or ethanol, at a temperature from about −78° C. to about 80° C. Preferably, this reaction is carried out in THF at about −78° C. Suitable bases for use in the aldol formation step include potassium carbonate (K₂CO₃), sodium carbonate (Na₂CO₃), sodium hydride (NaH), sodium methoxide, potassium-tert.-butoxide, lithium diisopropylamide, pyrrolidine and piperidine. Lithium diisopropylamide is preferred. Aldol condensations are described in “Modern Synthetic Reactions,” Herbert 0. House, 2d. Edition, W. A. Benjamin, Menlo Park, Calif., 629-682 (1972), J. Org. Chem., 49, 2455 (1984), and Tetrahedron, 38 (20), 3059 (1982).

Compounds of the formula IV wherein R³ is unsaturated can be converted to saturated analogues by hydrogenating the compounds containing a carbon-carbon double bond, using standard techniques that are well known to those skilled in the art. For example, reduction of the double bond may be effected with hydrogen gas (H₂), using catalysts such as palladium on carbon (Pd/C), palladium on barium sulfate (Pd/BaSO₄), platinum on carbon (Pt/C), or tris(triphenylphosphine) rhodium chloride (Wilkinson's catalyst), in an appropriate solvent such as methanol, ethanol, THF, dioxane or ethyl acetate, at a pressure from about 1 to about 5 atmospheres and a temperature from about 10° C. to about 60° C., as described in Catalytic Hydrogenation in Organic Synthesis, Paul Rylander, Academic Press Inc., San Diego, 31-63 (1979). The following conditions are preferred: Pd on carbon, methanol at 25° C. and 50 psi of hydrogen gas pressure. This method also provides for introduction of hydrogen isotopes (i.e., deuterium, tritium) by replacing ¹H₂ with ²H₂ or ³H₂ in the above procedure.

An alternative procedure employing the use of reagents such as ammonium formate and Pd/C in methanol at the reflux temperature under an inert atmosphere (I, nitrogen or argon gas) is also effective in reducing the carbon-carbon double bond of compounds of the formula I. Another alternative method involves selective reduction of the carbon-carbon bond. This can be accomplished using samarium and iodine or samarium iodide (SmI₂) in methanol or ethanol at about room temperature, as described by R. Yanada et. al., Synlett., 443-4 (1995).

Compounds of the formula V can be prepared by methods well known to those of ordinary skill in the art or are commercially available. Specifically, compounds of the formula Va and Vb (shown below) can be prepared by the method of Fray et al., (J. Org. Chem., 51, 4828-4833 (1986)) using an (S)-aldehyde of the formula

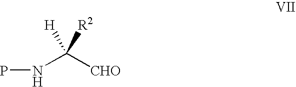

Compounds of the formula VII are prepared by reducing amino acids or amino esters to alcohols (Stanfield et al., J. Org. Chem. 46, 4799-4800 (1981), Soai et al., Bull. Chem. Soc. Jpn., 57, 2327 (1984)) followed by oxidation of the alcohols to aldehydes of the formula VII (Luly et al., J. Org. Chem., 53 (26), 6109-6112 (1988) and Denis et al., J. Org. Chem., 56 (24), 6939-6942 (1991).). Un-natural amino acids can be prepared according to the method of Myers et al., Tet. Lett. 36, (1995) and Myers et al. J. Am. Chem. Soc., 117, 8488-8489 (1995).

Alternatively, compounds of the formula V can also be made by the method of DeCamp et al., (Tetrahedron Lett., 32, 1867 (1991)).

Compounds of the formula Ia may be made by the method shown in Schemes 8 and 9.

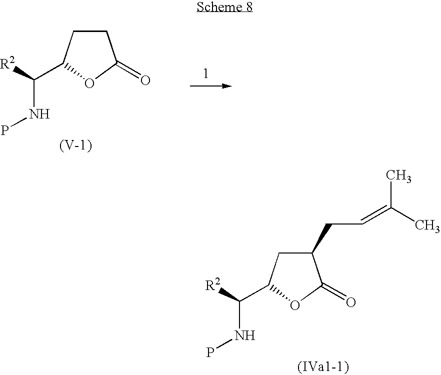

In step 1 of Scheme 8, the compound of the formula (IVa1-1) may be formed by reacting 4-halo-2-methyl-2-butene and a compound of the formula (v-1) in the presence of a base. Exemplary bases include lithium dialkyl amides such as lithium n-isopropyl-n-cyclohexylamide, lithium bis(trimethylsilyl)amide, lithium diisopropylamide, and potassium hydride. Suitable solvents include aprotic polar solvents such as ethers (such as tetrahydrofuran, glyme or dioxane), benzene, or toluene, preferably tetrahydrofuran. The aforesaid reaction is conducted at a temperature from about −78° C. to about 0° C., preferably at about −78° C. In one embodiment, alkylation of the lactone (v-1) is accomplished by reacting the lactone (v-1) with lithium bis(trimethylsilyl)amide and dimethylallyl bromide in tetrahydrofuran at a temperature from about −78° C. to about −50° C. Reaction times range from several hours or if an additive such as dimethyl imidazolidinone is present, the reaction may be complete in minutes.

Compounds of formula (IVa1-1) may be used to produce compounds of the formula (Ia-1) according to Scheme 9:

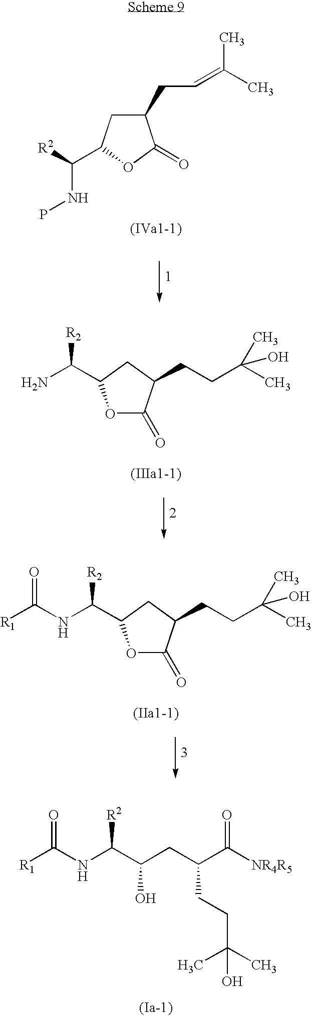

In step 1 of Scheme 9, a compound of the formula (IIIa1-1) is formed by reacting a compound of the formula (IVa1-1) with phosphoric acid. Preferably, this reaction occurs in any suitable solvent, such as non-alcoholic solvents. Two preferred solvents include tetrahydrofuran and dichloromethane. The reaction may take place at any suitable temperature, preferably from about −25° C. to about 120° C., more preferably from about 15° C. to about 40° C. Reaction time is dependent on temperature and batch size, amount other factors, but typically reaction time is from about 2 hours to about 14 hours.

Step 2 of Scheme 9 depicts coupling a compound IIIa1-1 with a compound having the formula R₁—CO—X to form a compound having the formula (IIa1-1). This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI), and diethylphosphorylcyanide. The coupling is conducted in an inert solvent, preferably an aprotic solvent, such as tetrahydrofuran, acetonitrile, dichloromethane, chloroform, or N,N-dimethylformamide. One preferred solvent is tetrahydrofuran. In one embodiment, quinoxaline acid is combined with CDI in anhydrous tetrahydrofuran and heated to provide the acyl imidazole. Compound IIIa1-1 is added to the acyl imidazole at room temperature to form the compound IIa1-1.

Step 3 of Scheme 9 includes reacting the compound of formula llal-1 with an amine having a formula NHR₄(R)₅ to form a compound of the formula (Ia-1). In one embodiment, the amine is ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa1-1 is dissolved in methanol which has been saturated with ammonia gas. In another embodiment, the compound IIa1-1 in methanol is treated with ammonium hydroxide in tetrahydrofuran at room temperature.

Scheme 10 represents an alternative method to form compounds of formula Ia-1 from compounds of formula IVa1-1.

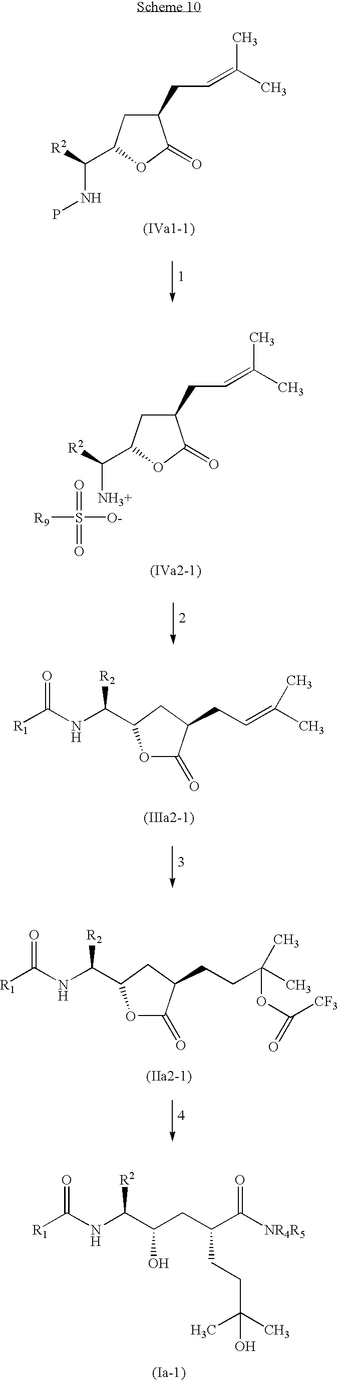

In step 1 of Scheme 10, a compound of the formula (IVa1-1) is reacted with a 5 compound of the formula R₉—SO₂—OH to form a compound of the formula (IVa2-1). Any suitable acidic deprotection reaction may be performed. In one example, an excess of p-toluenesulfonic acid hydrate in ethyl acetate is introduced to the compound IVa1-1 at room temperature. Suitable solvents include ethyl acetate, alcohols, tetrahydrofuran, and mixtures thereof. The reaction may proceed at ambient or elevated temperatures. Typically, the reaction is substantially complete within two and twelve hours. The resulting compound IVa2-1 may be crystallized and separated from the reaction mixture, and may be further purified to remove impurities by recrystallization from hot ethyl acetate.

In step 2 of Scheme 10, the compound IVa2-1 may be coupled with a compound having the formula R₁—CO—X to form a compound of the formula (IIIa2-1). This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI)/dimethylaminopyridine (DMAP), and diethylphosphorylcyanide. The coupling is conducted in an inert solvent, preferably an aprotic solvent, such as acetonitrile, dichloromethane, chloroform, or N,N-dimethylformamide. One preferred solvent is methylene chloride. In one embodiment, quinoxaline acid is combined with methylene chloride, oxalyl chloride and a catalytic amount of N,N-dimethylformamide to form an acid chloride complex. The compound IVa2-1 is added to the acid chloride complex followed by triethylamine at a temperature from about 0° C. to about 25° C. to form the compound IIIa2-1.

Step 3 of Scheme 10 includes reacting a compound IIIa2-1 with trifluoroacetic acid to produce a compound of the formula (IIa2-1). In one embodiment, the hydration with trifluoroacetic acid occurs in methylene chloride solution at room temperature. The hydration may take several hours to complete at room temperature. A catalytic amount of sulfuric acid can be added to the reaction solution to increase the rate of reaction.

Step 4 of Scheme 10 includes reacting the compound of formula IIa2-1 with an amine having a formula NHR₄(R)₅ to form a compound of the formula (Ia-1). In one embodiment, the amine is ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa2-1 is dissolved in methanol which has been saturated with ammonia gas. In another embodiment, the compound IIa2-1 in methanol is treated with ammonium hydroxide in tetrahydrofuran at room temperature.

Unless indicated otherwise, the pressure of each of the above reactions is not critical. Generally, the reactions will be conducted at a pressure of about one to about three atmospheres, preferably at ambient pressure (about one atmosphere).

The compounds of the formula I and Ia which are basic in nature are capable of forming a wide variety of different salts with various inorganic and organic acids. Although such salts must be pharmaceutically acceptable for administration to animals, it is often desirable in practice to initially isolate a compound of the formula I and Ia from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1,1′-methylene-bis-(2-hydroxy-3-naphthoate)] salts.

Those compounds of the formula I and Ia which are also acidic in nature, are capable of forming base salts with various pharmacologically acceptable cations. The chemical bases which are used as reagents to prepare the pharmaceutically acceptable base salts of this invention are those which form non-toxic base salts with the herein described acidic compounds of formula I and Ia. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. These salts are all prepared by conventional techniques by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Compounds of the formula I and Ia and their pharmaceutically acceptable forms (hereinafter also referred to, collectively, as “the active compounds”) are potent and selective inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment and prevention of the following disorders and conditions: autoimmune diseases (such as rheumatoid arthritis, Takayasu arthritis, psoriatic arthritis, juvenile arthritis, ankylosing spondylitis, type I diabetes (recent onset), lupus, inflammatory bowel disease, Chrohn's disease, optic neuritis, psoriasis, neuroimmunologic disease (multiple sclerosis (MS) primary progressive MS, secondary progressive MS, chronic progressive MS, progressive relapsing MS, relapsing remitting MS, worsening MS), polymyalgia rheumatica, uveitis, thyroiditis and vasculitis); fibrosis (such as pulmonary fibrosis (for example idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic inflammatory conditions including ocular inflammation, stenosis, lung inflammation (such as chronic bronchitis, chronic obstructive pulmonary disease, adult Respiratory Distress Syndrome, Respiratory Distress Syndrome of infancy, immune complex alveolitis), vascular inflammation resulting from tissue transplant or during restenosis (including, but not limited to, restenosis following angioplasty and/or stent insertion) and other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and chronic transplant rejection (including xeno-transplantation); HIV infectivity (coreceptor usage); granulomatous diseases (including sarcoidosis, leprosy and tuberculosis); Alzheimer's disease; chronic fatigue syndrome; pain; atherosclerosis; conditions associated with leptin production (such as obesity, cachexia, anorexia, type II diabetes, hyperlipidemia and hypergonadism); and sequelae associated with certain cancers such as multiple myeloma. This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the invention can be assessed according to procedures know to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

### Chemotaxis Assay

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1a (Peprotech, Inc., P.O. Box 275, Rocky Hill N.J.) or other test agonists, were placed into the lower chambers of the Boyden chamber. A polycarbonate filter was then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of >90% to be considered a valid assay.

All of the compounds of the invention that were tested had IC 50 of less than 25 μM, in the Chemotaxis assay.

The compositions of the present invention may be formulated in a conventional manner using one or more pharmaceutically acceptable carriers. Thus, the active compounds of the invention may be formulated for oral, buccal, intranasal, topical, transdermal, parenteral (e.g., intravenous, intramuscular or subcutaneous) ocular or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner. Moreover, quick dissolve tablets may be formulated for sublingual absorption.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch to provide for dry powder inhalation.

A proposed dose of the active compounds of the invention for oral, parenteral, nasal, or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents may be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, all of which are incorporated herein in their entireties for all purposes.

The compounds of the invention may also be utilized in combination therapy with other therapeutic agents such as those that inhibit immune cell activation and/or cytokine secretion or action (i.e. Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-Ig), BMS-224818 (CTLA4-Ig), RG-1046 (CTLA4-Ig), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, Anti-interleukin-6 receptor monoclonal antibody (MRA)), NSAIDS (aspirin, acetaminophen, naproxen, ibuprofen, ketoprofen, diclofenac and piroxicam), COX-2 inhibitors (Celecoxib, Valdecoxib, Rofecoxib, Parecoxib, Etoricoxib, L-745337, COX-189, BMS-347070, S-2474, JTE-522, CS-502, P-54, DFP), Glatiramer acetate, Interferon beta 1-a, Interferon beta 1-b, Mitoxantrone, Pimecrolimus, or agents that inhibit cell recruitment mechanisms (e.g. inhibitors of integrin upregulation or function) or alter leukocyte trafficking.

### EXPERIMENTAL

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Melting points are uncorrected. NMR data are reported in parts per million (8) and are referenced to the deuterium lock signal from the sample solvent (deuterochloroform unless otherwise specified). Chromatography refers to column chromatography performed using 32-63 mm silica gel and executed under nitrogen pressure (flash chromatography) conditions. Low Resolution Mass Spectra (LRMS) were recorded on either a Hewlett Packard 5989®, utilizing chemical ionization (ammonium), or a Fisons (or Micro Mass) Atmospheric Pressure Chemical Ionization (APCI) platform which uses a 50/50 mixture of acetonitrile/water with 0.1% formic acid as the ionizing agent. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein Information system GmbH (ISBN 3-89536-976-4). Note that all numbers provided herein are approximate, but effort have been made to ensure accuracy with respect to numbers (e.g., amounts, temperature, etc.); however some errors and deviations should be accounted for.

### Example 1

### Quinoline-3-carboxylic Acid (1(s)-cyclohexylmethyl-2(s)-hydroxy-6-methyl-4(r)-methylcarbamoyl-heptyl-6-enyl)-amide

### Method A

### Quinoline-3-carboxylic Acid {1-[4-(2-methylpropen-2-yl)-5-oxo-tetrahydrofuran-2-yl]-2-cyclohexyl-ethyl}-amide

To a solution of 1-{4-(2-methylpropen-2-yl)-[5-oxo-tetrahydrofuran-2-yl]-2-cyclohexyl-ethyl}-carbamic acid tert-butyl ester (302 mg, 0.83 mmol)(prepared according to the method of Fray, supra, except that (S)-2-(tert-butoxycarbonylamino)₃-cyclohexyl-1-propionaldehyde is the starting material aldehyde) in 15 mL of methylene chloride was added 1.5 mL of trifluoroacetic acid. The mixture was stirred at room temperature under a nitrogen atmosphere for 2 hours at which time the solvent was removed by azeotropic distillation under reduced pressure, using toluene as a cosolvent during the distillation. The resulting crude oil was dissolved in methylene chloride (5 mL) and quinoline-3-carboxylic acid (219 mg, 1.26 mmol), hydroxybenzotriazole (HOBT)(188 mg, 1.39 mmol), triethylamine (0.25 mL, 1.80 mmol) and N-3-dimethylaminopropyl-N′-ethylcarbodiimide (EDC)(248 mg, 1.29 mmol) was added. The resulting mixture was stirred at room temperature for 16 hours. The solution was transferred to a separatory funnel with 15 mL of methylene chloride and washed with 10% citric acid, saturated sodium bicarbonate and brine. The organic layer was dried over sodium sulfate and the solvent removed in vacuo. The remaining crude oil was purified by silica gel chromatography eluting with 3:1 hexanes:ethyl acetate to provide quinoline-3-carboxylic acid {1{S)-[4(R)-(2-methylpropen-2-yl)-5-oxo-tetrahydrofuran-2(S)-yl]-2-cyclohexyl-ethyl}-amide as a white foam (236 mg, 67%).

LRMS: 421 (MH+); ¹H NMR (300 MHz, CDCl₃): δ 0.90-1.89 (m, 13H), 1.63 (s, 3H), 2.03-2.14 (m, 2H), 2.38 (m, 2H), 2.48 (d, 1H, J=14.6 Hz), 2.73 (m, 1H), 4.63 (m, 2H), 4.69 (s, 1H), 4.79 (s, 1H), 6.9 (brs, 1H), 7.59 (t, 1H, J=7.8 Hz), 7.77 (t, 1H, J=8.4 Hz), 7.88 (d, 1H, J=8.3 Hz), 8.08 (d, 1H, J=8.4 Hz), 8.67 (s, 1H), 9.37 (d, 1H, J=2.1 Hz).

### Method B

### Quinoline-3-carboxylic Acid (1(s)-cyclohexylmethyl-2(s)-hydroxy-6-methyl-4(r)-methylcarbamoyl-heptyl-enyl)-amide

Methylamine was bubbled into a solution of the product from Method A (55 mg, 0.129 mmol) in methanol (2.5 mL). The solution was stirred for 2 hours at room temperature and the solvent was removed under reduced pressure to provide the title compound (57 mg, 98%) as a pure white solid.

LRMS: 453 (MH⁺), 421, 283, 173; ¹H NMR (300 MHz, CDCl₃): δ 0.82-1.87 (m, 13H), 1.65 (s, 3H), 2.13 (dd, 1H, J=14.1, 8.7 Hz), 2.38 (d, 1H, J=14.2 Hz), 2.71 (d, 3H, J=4.7 Hz), 2.74 (m, 1H), 3.77 (d, 1H, J=8.7), 4.23 (br, 1H), 4.69 (s, 1H), 4.72 (s, 1H), 5.03 (brs, 1H), 6.60 (q, 1H, J=4.7 Hz), 7.24 (d, 1H, J=9.3), 7.54 (t, 1H, J=7.1), 7.73 (t, 1H, J=7.1 Hz), 7.81 (d, 1H, J=7.1 Hz), 8.04 (d, 1H, J=8.4), 8.61 (d, 1H, J=1.9), 9.33 (s, 1H).

### Example 2

### Quinoxaline-2-carboxylic Acid (1(s)-benzyl-4(r)-benzylcarbamoyl-7-fluoro-2(s)-hydroxy-7-methyl-octyl)-amide Allylic Alkylation

### Method C

### {1(s)-[4(r)-(3-methyl-but-2-enyl)-5-oxo-tetrahydro-furan-2(s)-yl]-2-phenyl-ethyl}-carbamic Acid Tert-Butyl Ester

To a flame dried round bottom flask under a nitrogen atmosphere was' added tetrahydrofuran (40 mL) followed by 1,1,1,3,3,3-hexamethyldisilazane (8 mL, 37.8 mmol). The mixture was cooled to 0° C. and n-butyl lithium (14.5 mL of a 2.5 M solution in hexanes, 36.0 mmol) was added. The mixture was stirred for 15 minutes, then cooled to −78° C. in dry ice/acetone bath. {1(S)-[5-Oxo-tetrahydro-furan-2(S)-yl]-2-phenyl-ethyl}-carbamic acid tert-butyl ester (5 g, 16.4 mmol) (prepared by the method of Fray, J. Org. Chem., (51) 4828 (1986)) dissolved in tetrahydrofuran (50 mL) was added dropwise via syringe and stirring continued for 30 minutes. A solution of 4-bromo-2-methyl-2-butene (2.07 mL, 18.0 mmol) in 40 mL of THF was added dropwise via syringe. Stirring was continued for 3 hours during which time the temperature rose to −60° C. The mixture was quenched by slow addition of saturated, aqueous ammonium chloride (25 mL). Upon warming to room temperature, the solution was diluted with ether (300 mL) and transferred to a separatory funnel. The organic phase was washed with saturated aqueous citric acid (2×100 mL), saturated aqueous sodium bicarbonate (NaHCO₃)(2×100 mL), and 100 mL brine. The organic layer was dried over magnesium sulfate (MgSO₄) and the solvent removed under reduced pressure. Thin layer chromatography in 1:2 hexane/diethyl ether (Et₂O) revealed product with an R_{f} of 0.8. The resulting crude oil was chromatographed on silica gel (225 g) eluting with 2:1 hexanes/diethyl ether to provide 4.73 g (77%) of the title compound. TLC: 1:2 Hexanes/Et₂O R_{f}: 0.8. ¹H NMR (400 MHz, CDCl₃): δ 7.27 ppm (5H, m), 5.02 (1H, b), 4.52 (1H, d, J=9.3 Hz), 4.42 (1H, t, J=7.1 Hz), 3.98 (1H, dt, J=8.5, 7.8 Hz), 2.93 (2H, m), 2.88 (1H, b), 2.68 (1H, m), 2.41 (1H, m), 2.24 (1H, m), 1.92 (1H, m), 1.65 (3H, s), 1.58 (3H, s), 1.37 (9H, s).

### Method D

### 5(s)-(1(s)-amino-2-phenyl-ethyl)-3(r)-(3-fluoro-3-methyl-butyl)-dihydro-furan-2-one

To a solution of product from Method C (9.81 g, 26.3 mmol) in dry benzene (300 mL) was added HF-pyridine (88 mL). The resulting solution was stirred at ambient temperature for 4 hours, then transferred to a 4 L beaker. To this was added ice, and the pH was slowly adjusted to 8-9 by addition of 2 M aqueous sodium hydroxide (NaOH_{aq}). The mixture was extracted with ethyl acetate (EtOAc) and the organics dried over magnesium sulfate, and then filtered and concentrated. Chromatography on silica gel yielded the title compound (5.68 g, 74%).

### Method E

### Quinoxaline-2-carboxylic Acid {1(s)-[4(r)-(3-fluoro-3-methyl-butyl)-5-oxotetrahydro-furan-2(s)-yl]-2-phenyl-ethyl}-amide

To a solution of quinoxaline carboxylic acid (5.05 g, 29.0 mmol) in methylene chloride (100 mL) was added dimethylaminopyridine (DMAP) (3.55 g, 29.0 mmol) and EDCl (5.55 g, 29.0 mmol). The solution was stirred 10 minutes, then the product from Method D, above, (5.68 g, 19.4 mmol) was added in one portion. The solution was stirred for 12 hours, then diluted with diethyl ether and washed with saturated aqueous brine. The organics were dried over magnesium sulfate, and then filtered and concentrated. The crude product was purified by silica gel chromatography to yield the title compound (5.62 g, 64%).

### Method F

### Quinoxaline-2-carboxylic Acid (1(s)-benzyl-4(r)-benzylcarbamoyl-7-fluoro-2(s)-hydroxy-7-methyl-octyl)-amide

To a solution of the product from Method E (0.10 g, 0.22 mmol) in dioxane (2 mL) was added glacial acetic acid (0.038 mL, 0.66 mmol) and benzylamine (approx. 1 mL, excess). The resulting solution was warmed to reflux for 1 hour, cooled to ambient temperature and diluted with water. The solution was extracted with ethyl acetate and the combined organics were dried over magnesium sulfate (MgSO₄), filtered and concentrated. Chromatography on silica gel, followed by recrystallization from methylene chloride/hexanes gave the title compound (0.068 g, 56%). m.p. 183-184° C.

### Example 3

### Method F′

### Quinoxaline-2-carboxylic Acid (1-benzyl-7-fluoro-2-hydroxy-4-hydroxycarbamoyl-7-methyl-octyl)-amide

Hydroxylamine hydrochloride (1.55 g, 22.4 mmol) and KOH (1.51 g, 26.7 mmol) were combined in anhydrous methanol (20 mL) and stirred for 30 minutes under a dry nitrogen atmosphere, and then filtered. To the resulting filtrate was added the product from Method E (500 mg, 1.17 mmol) and the reaction mixture was stirred for 16 hours at room temperature. The solvent was removed in vacuo and the residue solvated in EtOAc (50 mL) and transferred to a separated funnel. The organic layer was washed with water and brine and dried (MgSO4). After filtration the solvent was removed in vacuo and the remaining residue recrystallized (methylene chloride/Hexanes) to give a pale yellow solid (330 mg, 58%) m.p. 165-166° C.

### Example 4

### Quinoxaline-2-carboxylic Acid (1(s)-benzyl-4(r)-carbamoyl-2(s)-hydroxy-7-methyl-octyl)-amide

### Method G

### Alkene Hydrogenation

### {1 (s)-[4(r)-(3-methyl-butyl)-5-oxo-tetrahydro-furan-2(s)-yl]-2-phenyl-ethyl}-carbamic Acid Tert-Butyl Ester

The product from Method C, from Example 2 above, (3.0 g, 8.04 mmol) was placed in a 250 mL Parr Shaker bottle and dissolved in ethanol (50 mL). Under a nitrogen atmosphere, Palladium (Pd) on activated carbon (0.30 g, 10% Pd content) was added to the solution. The mixture was placed on a Parr Shaker hydrogenator at 50 psi for 5 hours at room temperature. The hydrogenation mixture was diluted with ethyl acetate and then poured through a Celite® pad while washing copiously with ethyl acetate. The solvent of the filtrate was removed in vacuo to yield the title compound, 2.63 g (88%).

¹H NMR (400 MHz, CDCl₃): δ 7.27 (5H, m), 4.54 (1H, d, J=9.8 Hz), 4.46 (1H, t, J=6.9), 4.0 (1H, dt), 2.89 (2H, d, J=8.1), 2.57 (1H, m), 2.32 (1H, b), 1.89 (1H, m), 1.79 (1H, m), 1.52 (2H, m), 1.37 (9H, s), 1.23 (2H, m), 0.86 (6H, d, J=6.6 Hz).

The product from Method G was converted into the title compound by procedures analogous to those of Methods A and B except that quinoline-3-carboxylic acid is replaced with quinoxaline-2-carboxylic acid and methylamine is replaced with ammonia gas to yield 0.095 g (72%) of the title compound.

¹H NMR (400 MHz, CDCl₃): δ 9.61(1H, s), 8.32 (1H, d, J=8.9 Hz), 8.16 (2H, m), 7.86 (2H, m), 7.28 (10H, m), 7.19 (1H, m), 5.70 (1H, b), 5.29 (1H, b), 4.27 (1H, m), 8.21 (1H, d, J=4.4 Hz), 3.91 (1H, m), 3.11 (2H, m), 2.46 (1H, m), 1.74 (1H, t, J=6.4 Hz), 1.61 (1H, m), 1.42 (2H, m), 1.17 (1H, m), 1.09 (1H, m), 0.81 (3H, d, J=7.1 Hz), 0.79 (3H, d, J=7.1 Hz). ¹³C NMR (100 MHz, CDCl₃): d 179.11, 163.73, 143.90, 143.76, 143.15, 140.28, 137.96, 131.68, 130.84, 129.84, 129.44, 129.25, 128.58, 126.60, 68.55, 55.90, 43.44, 38.39, 36.90, 36.70, 29.77, 28.03, 22.42

### Example 5

### Quinoxaline-2-carboxylic Acid 1(s)-benzyl-4(r)-carbamoyl-2(s)-hydroxy-7,7-dimethyl-octyl)-amide

### Method H

### Triflate Alkylation

### {1-[4-(3.3-dimethyl-butyl)-5-oxo-tetrahydro-furan-2-yl]-2-phenyl-ethyl}-carbamic Acid Tert-Butyl Ester

To a flame dried round bottom flask under a nitrogen atmosphere was added terahydrofuran (THF) (2 mL) and 1,1,1,3,3,3 hexamethyldisilazane (0.82 mL, 3.88 mmol). The mixture was cooled to 0° C. and n-butyl lithium (1.48 mL of a 2.5 M solution in hexanes, 3.72 mmol) was added dropwise via syringe. The mixture was stirred for 15 minutes and then cooled to −78° C. {1 (S)-[5-Oxo-tetrahydro-furan-2(S)-yl]-2-phenyl-ethyl}-carbamic acid tert-butyl ester (0.52 g, 1.69 mmol prepared by the method of Fray, supra) dissolved in tetrahydrofuran (2 mL) was slowly added to the solution via syringe and the solution was stirred for 1 hour. A solution of the desired triflate, i.e. 3,3-dimethylbutyl triflate (0.92 g, 3.37 mmol)(prepared according to the method of Beard, et al., J Org Chem., 38, 3673 (1973)) in tetrahydrofuran (2 mL) was added dropwise via syringe and the mixture was stirred for 2 hours at −78° C. The mixture was quenched by addition of saturated aqueous ammonium chloride (NH₄Cl) (25 mL). Upon warming to room temperature, the mixture was diluted with ethyl acetate (40 mL), transferred to a separatory funnel, and washed with saturated aqueous NH₄Cl (2×40 mL), saturated NaHCO₃ (2×40 mL), and brine (40 mL). The organic layers were dried (MgSO₄) and the solvent removed under reduced pressure. The resulting crude oil was chromatographed on silica gel (25 g) eluting with 100 mL 5:1 hexanes/ethyl acetate followed by 400 mL 4:1 hexanes/ethyl acetate. This provided 0.36 g (50%) of the title compound.

TLC: (4:1 hexanes/ethyl acetate) R_{f}: 0.3. ¹H NMR (400 MHz, CDCl₃): δ 7.25 (m, 7H), 6.92 (t, 1H, J=7.5 Hz), 6.85 (d, 2H, J=8.1 Hz), 4.67 (d, 2H, J=6.0 Hz), 4.49 (t, 1H, J=9.6 Hz), 4.06 (m, 3H), 2.89 (m, 3H), 2.43 (m, 1H), 2.26 (m, 1H), 2.05 (m, 1H), 1.95 (m, 1H), 1.37 (s, 9H).

The product of Method H was converted to the title compound by procedures analogous to those of Methods A and B, from Example 1, except that quinoline-3-carboxylic acid is replaced with quinoxaline-2-carboxylic acid and methylamine is replaced with ammonia gas.

### Example 6

### Quinoxaline-2-carboxylic Acid [1(s)-benzyl-4(s)-carbamoyl-2(s)-hydroxy-4-(1-hydroxy-cyclohexyl)-butyl]-amide and

### Quinoxaline-2-carboxylic Acid [1(s)-benzyl-4(r)-carbamoyl-2(s)-hydroxy-4-(1-hydroxy-cyclohexyl)-butyl]-amide

### Method I

### {1 (s)-[4(s)-(1-hydroxy-cyclohexyl)-5-oxo-tetrahydro-furan-2(s)-yl]-2-phenyl-ethyl}-carbamic Acid Tert-Butyl Ester

To a solution of diisopropylamine (0.90 mL, 6.88 mmol) in THF (10 mL) at 0° C. was added a solution of n-butyl lithium (2.7 mL, 6.71 mmol, 2.5 M in hexanes). The solution was stirred for 15 minutes, then cooled to −78° C. To this was added dropwise a solution of {1 (S)-[5-Oxo-tetrahydro-furan-2(S)-yl]-2-phenyl-ethyl}-carbamic acid tert-butyl ester (1.0 g, 3.27 mmol prepared as in example 2, method C) in tetrahydrofuran (10 mL) and the reaction was stirred an additional 30 minutes. To this was added the appropriate ketone, e.g., cyclohexanone) (0.37 mL, 3.60 mmol), and the solution was warmed to ambient temperature. The reaction was quenched by addition of saturated aqueous bicarbonated NaHCO₃) solution and the mixture extracted with diethyl ether. The combined organics were dried over magnesium sulfate (MgSO4), filtered and concentrated. Chromatography on silica gel gave a mixture of separable diastereomers of {[1 (S)-[4(S)-(1-hydroxy-cyclohexyl)-5-oxo-tetrahydro-furan-2(S)-yl]-2-phenyl-ethyl}-carbamic acid tert-butyl ester (0.687 g) and {1 (S)-[4(R)-(1-hydroxy-cyclohexyl)-5-oxo-tetrahydro-furan-2(S)-yl]-2-phenyl-ethyl}-carbamic acid tert-butyl ester (0.269 g) in 67% overall yield.

The products from Method I were converted to the title compounds by procedures analogous to those of Methods A and B, from Example 1, except that quinoline-3-carboxylic acid is replaced with quinoxaline-2-carboxylic acid and methylamine is replaced with ammonia gas.

### Example 7

### Fluoro-quinoline-3-carboxylic Acid (1(s)-benzyl-4(s)-carbamoyl-4-cyclohexyl-2(s)-hydroxy-butyl)-amide and

### Fluoro-quinoline-3-carboxylic Acid (1(s)-benzyl-4(r)-carbamoyl-4-cyclohexyl-2(s)-hydroxy-butyl)-amide

### Method J

### {1(s)-[4(s)-(1-hydroxy-cyclohexyl)-5-oxo-tetrahydro-furan-2(s)-yl]-2-phenyl-ethyl}-carbamic Acid Tert-Butyl Ester

To a solution of the title compound from Method I, Example 5, (1.38 g, 3.42 mmol) in benzene (40 mL) was added (methoxycarbonylsulfamoyl)-triethylammonium hydroxide, inner salt (Burgess reagent) (1.30 g, 5.47 mmol) and the solution was warmed to reflux for 2 hours. The reaction was diluted with diethyl ether and washed with saturated aqueous brine. The organics were dried over magnesium sulfate, filtered and concentrated to give the crude elimination product. This was directly dissolved in 5:1 tetrahydrofuran/methanol (THF/MeOH)(30 mL) and transferred to a Parr flask containing 10% palladium on carbon (Pd/C) (1 g). The mixture was hydrogenated at 35 psi for 1.5 hours, then filtered through a pad of Celite and the filtrate concentrated. Chromatography on silica gel yielded the title compound as a mixture of separable diastereomers {1(S)-[4(S)-(1-hydroxy-cyclohexyl)-5-oxo-tetrahydro-furan-2(S)-yl]-2-phenyl-ethyl}-carbamic acid tert-butyl ester (0.53 g) and {1 (S)-[4(R)-(1-hydroxy-cyclohexyl)-5-oxo-tetrahydro-furan-2(S)-yl]-2-phenyl-ethyl}-carbamic acid tert-butyl ester (0.29 g) in 62% overall yield.

The products from Method J were converted to the title compounds by procedures analogous to those of Methods A and B, from Example 1, except that quinoline-3-carboxylic acid is replaced with quinoxaline-2-carboxylic acid and methylamine is replaced with ammonia gas.

### Examples 8-312

The compounds from Table 1 were prepared according to the methods described above, substituting where appropriate the correct R² aldehyde, R³ group (such as allylic halide, alkyl triflate, ketone, etc.), R¹ carboxylic acid or R⁴ and R⁵ amine where appropriate.

TABLE 1

| EXAM- | | | |
|---|---|---|---|
| PLE | NAME | M.P. (° C.) | LRMS |
| 8. | Quinoxaline-2-carboxylic acid | | 455 |
| | 1(S)-cyclohexylmethyl-2(S)-hydroxy-6- | | |
| | methyl-4(R)-methylcarbamoyl-heptyl)- | | |
| | amide | | |
| 9. | Quinoxaline-2-carboxylic acid | | |
| | (6-chloro-1-cyclohexylmethyl-2(S)- | | |
| | hydroxy-4(S)-methylcarbamoyl-hept-6- | | |
| | enyl)-amide | | |
| 10. | Quinoline-3-carboxylic acid | 155-157 | 414 |
| | (2(S)-hydroxy-1(S)-isobutyl-6-methyl- | | |
| | 4(R)-methylcarbamoyl-heptyl)-amide | | |
| 11. | Quinoxaline-2-carboxylic acid | 69-71 | 415 |
| | 1(S)-sec-butyl-2(S)-hydroxy-6-methyl- | | |
| | 4(R)-methylcarbamoyl-heptyl)-amide | | |
| 12. | Quinoline-3-carboxylic acid | | 452 |
| | 1(S)-cyclohexylmethyl-2(S)-hydroxy-6- | | |
| | methyl-4(R)-methylcarbamoyl-hept-6- | | |
| | enyl)-amide | | |
| 13. | Quinoxaline-2-carboxylic acid | | 453 |
| | 1(S)-cyclohexylmethyl-2(S)-hydroxy-6- | | |
| | methyl-4(R)-methylcarbamoyl-hept-6- | | |
| | enyl)-amide | | |
| 14. | N-1(S)-Cyclohexylmethyl-2(S)-hydroxy- | 115-119 | |
| | 6-methyl-4(R)-methylcarbamoyl- | | |
| | heptyl)-5-phenyl-nicotinamide | | |
| 15. | Quinoline-3-carboxylic acid 1(S)- | 162-163 | |
| | benzyl-2(S)-hydroxy-6-methyl-4(R)- | | |
| | methylcarbamoyl-heptyl)-amide | | |
| 16. | Quinoxaline-2-carboxylic acid | | 467 |
| | 1(S)-cyclohexylmethyl-4(R)- | | |
| | dimethylcarbamoyl-2(S)-hydroxy-6- | | |
| | methyl-hept-6-enyl)-amide | | |
| 17. | Quinoline-3-carboxylic acid | 171-175 | 453, |
| | 1(S)-cyclohexylmethyl-2(S)-hydroxy-6- | | 436 |
| | methyl-4(S)-methylcarbamoyl-heptyl)- | | |
| | amide | | |
| 18. | Quinoxaline-2-carboxylic acid | | 455, |
| | 1(S)-cyclohexylmethyl-2(S)-hydroxy-6- | | 437 |
| | methyl-4(S)-methylcarbamoyl-heptyl)- | | |
| | amide | | |
| 19. | Isoquinoline-4-carboxylic acid | 180-182 | 454 |
| | 1(S)-cyclohexylmethyl-2(S)-hydroxy-6- | | |
| | methyl-4(S)-methylcarbamoyl-heptyl)- | | |
| | amide | | |
| 20. | Quinoline-3-carboxylic acid | 186-188 | 440, |
| | (4(R)-carbamoyl-1(S)-cyclohexylmethyl- | | 478, |
| | 2(S)-hydroxy-6-methyl-heptyl)-amide | | 423 |
| 21. | Quinoline-3-carboxylic acid (5- | 170.5-172.5 | 494 |
| | cyclohexyl-1(S)-cyclohexylmethyl-2(S)- | | |
| | hydroxy-4(R)-methylcarbamoyl-pentyl)- | | |
| | amide | | |
| 22. | Quinoline-3-carboxylic acid 1(S)- | | 454 |
| | cyclohexylmethyl-2(S)-hydroxy-6- | | |
| | methyl-4(R)-methylcarbamoyl-heptyl)- | | |
| | amide | | |
| 23. | Quinoline-3-carboxylic acid | 200-201.5 | 454 |
| | 1(S)-cyclohexylmethyl-2(S)-hydroxy-6- | | |
| | methyl-4(S)-methylcarbamoyl-heptyl)- | | |
| | amide | | |
| 24. | Quinoline-3-carboxylic acid | 199-200.5 | 488 |
| | 1(S)-cyclohexylmethyl-2(S)-hydroxy- | | |
| | 4(R)-methylcarbamoyl-5-phenyl- | | |
| | pentyl)-amide | | |
| 25. | Quinoxaline-2-carboxylic acid | 109-110.5 | 489 |
| | 1(S)-cyclohexylmethyl-2(S)-hydroxy- | | |
| | 4(R)-methylcarbamoyl-5-phenyl- | | |
| | pentyl)-amide | | |
| 26. | Quinoline-3-carboxylic acid | 142-144 | 490, |
| | 1(S)-benzyl-4(R)-butylcarbamoyl-2(S)- | | 417 |
| | hydroxy-6-methyl-heptyl)-amide | | |
| 27. | Quinoline-3-carboxylic acid | 148-150 | 488, |
| | 1(S)-benzyl-4(R)-cyclobutylcarbamoyl- | | 417 |
| | 2(S)-hydroxy-6-methyl-heptyl)-amide | | |
| 28. | Quinoline-3-carboxylic acid | 158-162 | 524, |
| | 1(S)-benzyl-4(R)-benzylcarbamoyl- | | 417 |
| | 2(S)-hydroxy-6-methyl-heptyl)-amide | | |
| 29. | Quinoline-3-carboxylic acid | 174-179 | 474 |
| | 1(S)-benzyl-4(R)- | | |
| | cyclopropylcarbamoyl-2(S)-hydroxy-6- | | |
| | methyl-heptyl)-amide | | |
| 30. | Quinoline-3-carboxylic acid | 190-192.5 | 448 |
| | 1(S)-benzyl-2(S)-hydroxy-6-methyl- | | |
| | 4(S)-methylcarbamoyl-heptyl)-amide | | |
| 31. | Quinoline-3-carboxylic acid | 175-176 | 462 |
| | 1(S)-benzyl-4(R)-ethylcarbamoyl-2(S)- | | |
| | hydroxy-6-methyl-heptyl)-amide | | |
| 32. | Quinoline-3-carboxylic acid | | 476 |
| | 1(S)-benzyl-2(S)-hydroxy-6-methyl- | | |
| | 4(R)-propylcarbamoyl-heptyl)-amide | | |
| 33. | Quinoline-3-carboxylic acid | 158-162 | 478 |
| | [1-benzyl-2(S)-hydroxy-4(R)-(2- | | |
| | hydroxy-ethylcarbamoyl)-6-methyl- | | |
| | heptyl]-amide | | |
| 34. | Cinnoline-4(R)-carboxylic acid | 185-186.5 | 449 |
| | 1(S)-benzyl-2(S)-hydroxy-6-methyl- | | |
| | 4(R)-methylcarbamoyl-heptyl)-amide | | |
| 35. | Isoquinoline-4-carboxylic acid | 200-201 | 448 |
| | 1(S)-benzyl-2(S)-hydroxy-6-methyl- | | |
| | 4(R)-methylcarbamoyl-heptyl)-amide | | |
| 36. | Quinoxaline-2-carboxylic acid | 166-167 | 449 |
| | 1(S)-benzyl-2(S)-hydroxy-6-methyl- | | |
| | 4(R)-methylcarbamoyl-heptyl)-amide | | |
| 37. | N-1(S)-Benzyl-2(S)-hydroxy-6-methyl- | 184.5-185.5 | 478 |
| | 4(R)-methylcarbamoyl-heptyl)-5- | | |
| | bromo-nicotinamide | | |
| 38. | Quinoline-3-carboxylic acid | | 454 |
| | 1(R)-cyclohexylmethyl-2(R)-hydroxy-6- | | |
| | methyl-4(S)-methylcarbamoyl-heptyl)- | | |
| | amide | | |
| 39. | Quinoxaline-2-carboxylic acid | 196-197 | 554 |
| | [1(S)-(4-benzyloxy-benzyl)-2(S)- | | |
| | hydroxy-6-methyl-4(R)- | | |
| | methylcarbamoyl-heptyl]-amide, | | |
| 40. | Quinoline-3-carboxylic acid | 178-179 | 555 |
| | [1(S)-(4-benzyloxy-benzyl)-2(S)- | | |
| | hydroxy-6-methyl-4(R)- | | |
| | methylcarbamoyl-heptyl]-amide | | |
| 41. | Isoquinoline-1-carboxylic acid | 178-179 | 448 |
| | 1(S)-benzyl-2(S)-hydroxy-6-methyl- | | |
| | 4(R)-methylcarbamoyl-heptyl)-amide | | |
| 42. | Quinoline-4-carboxylic acid | 189-192 | 448 |
| | 1(S)-benzyl-2(S)-hydroxy-6-methyl- | | |
| | 4(R)-methylcarbamoyl-heptyl)-amide | | |
| 43. | Quinoline-6-carboxylic acid | 165-167 | 448 |
| | 1(S)-benzyl-2(S)-hydroxy-6-methyl- | | |
| | 4(R)-methylcarbamoyl-heptyl)-amide | | |
| 44. | Quinoline-3-carboxylic acid | 220.5-222.5 | 464 |
| | [2(S)-hydroxy-1(S)-(4-hydroxy-benzyl)- | | |
| | 6-methyl-4(R)-methylcarbamoyl- | | |
| | heptyl]-amide | | |
| 45. | Quinoline-2-carboxylic acid | 160-161.5 | 449 |
| | 1(S)-benzyl-2(S)-hydroxy-6-methyl- | | |
| | 4(R)-methylcarbamoyl-heptyl)-amide | | |
| 46. | Naphthalene-2-carboxylic acid | 218-220 | 447 |
| | 1(S)-benzyl-2(S)-hydroxy-6-methyl- | | |
| | 4(R)-methylcarbamoyl-heptyl)-amide | | |
| 47. | Quinoline-3-carboxylic acid | 172-174 | 486 |
| | 1(S)-benzyl-5-cyclohex-1-enyl-2(S)- | | |
| | hydroxy-4(R)-methylcarbamoyl-pentyl)- | | |
| | amide | | |
| 48. | Quinoline-3-carboxylic acid | 153-154 | 504 |
| | [1(S)-benzyl-2(S)-hydroxy-6-methyl- | | |
| | 4(R)-(3-methyl-butylcarbamoyl)-heptyl]- | | |
| | amide | | |
| 49. | Quinoxaline-2-carboxylic acid | 157-163 | 449 |
| | 1(S)-benzyl-2(S)-hydroxy-6-methyl- | | |
| | 4(S)-methylcarbamoyl-heptyl)-amide | | |
| 50. | Trifluoro-methanesulfonic acid | 168-170 | 596 |
| | 4-{3(S)-hydroxy-7-methyl-5(R)- | | |
| | methylcarbamoyl-2(S)-[(quinoline-3- | | |
| | carbonyl)-amino]-octyl}- | | |
| | phenyl ester | | |
| 51. | Trifluoro-methanesulfonic acid | | 597 |
| | 4-{3(S)-hydroxy-7-methyl-5(R)- | | |
| | methylcarbamoyl-2(S)-[(quinoxaline- | | |
| | 2-carbonyl)-amino]-octyl}-phenyl ester | | |
| 52. | Quinoline-3-carboxylic acid | 185-187 | 488 |
| | 1(S)-benzyl-5-cyclohexyl-2(S)-hydroxy- | | |
| | 4(R)-methylcarbamoyl-pentyl)-amide | | |
| 53. | Quinoxaline-2-carboxylic acid | 132-134 | 489, |
| | 1(S)-benzyl-5-cyclohexyl-2(S)-hydroxy- | | 471 |
| | 4(R)-methylcarbamoyl-pentyl)-amide | | |
| 54. | Isoquinoline-3-carboxylic acid | 150.5-151.5 | 488 |
| | 1(S)-benzyl-5-cyclohexyl-2(S)-hydroxy- | | |
| | 4(R)-methylcarbamoyl-pentyl)-amide | | |
| 55. | N-1(S)-Benzyl-5-cyclohexyl-2(S)- | 199-200.5 | 518 |
| | hydroxy-4(R)-methylcarbamoyl-pentyl)- | | |
| | 5-bromo-nicotinamide | | |
| 56. | Quinoline-3-carboxylic acid 1(S)- | | 472 |
| | benzyl-2(S)-hydroxy-6-methyl-4(R)- | | |
| | prop-2-ynylcarbamoyl-heptyl)-amide | | |
| 57. | Quinoline-3-carboxylic acid | | 456, |
| | 1(S)-cyclohexylmethyl-2(S)-hydroxy- | | 438, |
| | 4(R)-hydroxycarbamoyl-6-methyl- | | 423 |
| | heptyl)-amide | | |
| 58. | Quinoline-3-carboxylic acid 2(S)- | 176-177 | 478 |
| | hydroxy-1(S)-(4-methoxy-benzyl)-6- | | |
| | methyl-4(R)-methylcarbamoyl-heptyl]- | | |
| | amide | | |
| 59. | Isoquinoline-3-carboxylic acid (5- | 205-207 | 494 |
| | cyclohexyl-1(S)-cyclohexylmethyl-2(S)- | | |
| | hydroxy-4(R)-methylcarbamoyl-pentyl)- | | |
| | amide, | | |
| 60. | 5-Bromo-N-(5-cyclohexyl-1(S)- | 173.5-175 | 444 |
| | cyclohexylmethyl-2(S)-hydroxy-4(R)- | | |
| | methylcarbamoyl-pentyl)-nicotinamide | | |
| 61. | Quinoxaline-2-carboxylic acid | | 479 |
| | [2(S)-hydroxy-1(S)-(4-methoxy-benzyl)- | | |
| | 6-methyl-4(R)-methylcarbamoyl- | | |
| | heptyl]-amide | | |
| 62. | Isoquinoline-4-carboxylic acid | 220.5-224 | 494 |
| | (5-cyclohexyl-1(S)-cyclohexylmethyl- | | |
| | 2(S)-hydroxy-4(R)-methylcarbamoyl- | | |
| | pentyl)-amide | | |
| 63. | Quinoline-2-carboxylic acid | 120-122 | 488 |
| | 1(S)-benzyl-5-cyclohexyl-2(S)-hydroxy- | | |
| | 4(R)-methylcarbamoyl-pentyl)-amide | | |
| 64. | lsoquinoline-4-carboxylic acid | 177-180 | 488 |
| | 1(S)-benzyl-5-cyclohexyl-2(S)-hydroxy- | | |
| | 4(R)-methylcarbamoyl-pentyl)-amide, | | |
| 65. | Quinoxaline-2-carboxylic acid | 170-172 | 465 |
| | [2(S)-hydroxy-1(S)-(4-hydroxy-benzyl)- | | |
| | 6-methyl-4(R)-methylcarbamoyl- | | |
| | heptyl]-amide, | | |
| 66. | Quinoxaline-2-carboxylic acid | | 496 |
| | (5-cyclohexyl-1(S)-cyclohexylmethyl- | | |
| | 2(S)-hydroxy-4(R)-methylcarbamoyl- | | |
| | pentyl)-amide | | |
| 67. | Quinoline-3-carboxylic acid | 212.5-213.5 | 482 |
| | [1(S)-(4-chloro-benzyl)-2(S)-hydroxy-6- | | |
| | methyl-4(R)-methylcarbamoyl-heptyl]- | | |
| | amide | | |
| 68. | Quinoxaline-2-carboxylic acid | | 483 |
| | [1(S)-(4-chloro-benzyl)-2(S)-hydroxy-6- | | |
| | methyl-4(R)-methylcarbamoyl-heptyl]- | | |
| | amide | | |
| 69. | Quinoline-3-carboxylic acid | 173.5-175 | 468, |
| | 1(S)-cyclohexylmethyl-2(S)-hydroxy-7- | | 450 |
| | methyl-4(R)-methylcarbamoyl-octyl)- | | |
| | amide | | |
| 70. | Quinoxaline-2-carboxylic acid | 78-80 | 470 |
| | 1(S)-cyclohexylmethyl-2(S)-hydroxy-7- | | |
| | methyl-4(R)-methylcarbamoyl-octyl)- | | |
| | amide | | |
| 71. | Quinoline-3-carboxylic acid | 198-201 | 522 |
| | [1(S)-(4-chloro-benzyl)-5-cyclohexyl- | | |
| | 2(S)-hydroxy-4(R)-methylcarbamoyl- | | |
| | pentyl]-amide | | |
| 72. | Quinoxaline-2-carboxylic acid | | 523 |
| | [1(S)-(4-chloro-benzyl)-5-cyclohexyl- | | |
| | 2(S)-hydroxy-4(R)-methylcarbamoyl- | | |
| | pentyl]-amide | | |
| 73. | Quinoline-2-carboxylic acid | | 522 |
| | [1(S)-(4-chloro-benzyl)-5-cyclohexyl- | | |
| | 2(S)-hydroxy-4(R)-methylcarbamoyl- | | |
| | pentyl]-amide | | |
| 74. | Benzofuran-2-carboxylic acid | 181-183 | 437 |
| | 1(S)-benzyl-2(S)-hydroxy-6-methyl- | | |
| | 4(R)-methylcarbamoyl-heptyl)-amide | | |
| 75. | N-1(S)-Benzyl-2(S)-hydroxy-6-methyl- | 195-196 | 466, |
| | 4(R)-methylcarbamoyl-heptyl)-5,6- | | 432 |
| | dichloro-nicotinamide | | |
| 76. | Quinoline-3-carboxylic acid | 188-190 | 462 |
| | 1(S)-benzyl-2(S)-hydroxy-7-methyl- | | |
| | 4(R)-methylcarbamoyl-octyl)-amide | | |
| 77. | N-1(S)-Benzyl-2(S)-hydroxy-7-methyl- | 188-189 | 490 |
| | 4(R)-methylcarbamoyl-octyl)-5-bromo- | | |
| | nicotinamide | | |
| 78. | 5,6,7,8-Tetrahydro-quinoline-3- | 142.5-144.5 | 452 |
| | carboxylic acid | | |
| | 1(S)-benzyl-2(S)-hydroxy-6-methyl- | | |
| | 4(R)-methylcarbamoyl-heptyl)-amide | | |
| 79. | Quinoxaline-2-carboxylic acid | 147-149 | 463 |
| | 1(S)-benzyl-2(S)-hydroxy-7-methyl- | | |
| | 4(R)-methylcarbamoyl-octyl)-amide | | |
| 80. | Quinoline-2-carboxylic acid | 156-158 | 462 |
| | 1(S)-benzyl-2(S)-hydroxy-7-methyl- | | |
| | 4(R)-methylcarbamoyl-octyl)-amide, | | |
| 81. | lsoquinoline-4-carboxylic acid | 199-202 | 462 |
| | 1(S)-benzyl-2(S)-hydroxy-7-methyl- | | |
| | 4(R)-methylcarbamoyl-octyl)-amide | | |
| 82. | Quinoxaline-2-carboxylic acid | | 517, |
| | [1(S)-(3,4-dichloro-benzyl)-2(S)- | | 483 |
| | hydroxy-6-methyl-4(R)- | | |
| | methylcarbamoyl-heptyl]-amide | | |
| 83. | Benzo[b]thiophene-2-carboxylic acid | 179-181 | 453 |
| | 1(S)-benzyl-2(S)-hydroxy-6-methyl- | | |
| | 4(R)-methylcarbamoyl-heptyl)-amide | | |
| 84. | 2-Methyl-quinoline-3-carboxylic acid | 225-226.5 | 462 |
| | 1(S)-benzyl-2(S)-hydroxy-6-methyl- | | |
| | 4(R)-methylcarbamoyl-heptyl)-amide | | |
| 85. | 6,7-Dimethoxy-quinoline-3-carboxylic | 211-214 | 508 |
| | acid | | |
| | 1(S)-benzyl-2(S)-hydroxy-6-methyl- | | |
| | 4(R)-methylcarbamoyl-heptyl)-amide | | |
| 86. | 6,7-Difluoro-quinoline-3-carboxylic acid | 187-189 | 484, |
| | 1(S)-benzyl-2(S)-hydroxy-6-methyl- | | 466 |
| | 4(R)-methylcarbamoyl-heptyl)-amide | | |
| 87. | 1H-Benzoimidazole-2-carboxylic acid | 136-140 | 437 |
| | 1(S)-benzyl-2(S)-hydroxy-6-methyl- | | |
| | 4(R)-methylcarbamoyl-heptyl)-amide | | |
| 88. | 5-Methyl-pyrazine-2-carboxylic acid | 171.5-172.5 | 413 |
| | 1(S)-benzyl-2(S)-hydroxy-6-methyl- | | |
| | 4(R)-methylcarbamoyl-heptyl)-amide | | |
| 89. | Quinoline-3-carboxylic acid | 184-186 | 466 |
| | [1(S)-(4-fluoro-benzyl)-2(S)-hydroxy-6- | | |
| | methyl-4(R)-methylcarbamoyl-heptyl]- | | |
| | amide | | |
| 90. | Quinoxaline-2-carboxylic acid | 153-156 | 467 |
| | [1(S)-(4-fluoro-benzyl)-2(S)-hydroxy-6- | | |
| | methyl-4(R)-methylcarbamoyl-heptyl]- | | |
| | amide | | |
| 91. | 5-Chloro-1H-indole-2-carboxylic acid | 245-247 | 470 |
| | 1(S)-benzyl-2(S)-hydroxy-6-methyl- | | |
| | 4(R)-methylcarbamoyl-heptyl)-amide | | |
| 92. | Quinoxaline-2-carboxylic acid | 194-194.5 | 449, |
| | 1(S)-benzyl-4(R)-carbamoyl-2(S)- | | 432 |
| | hydroxy-7-methyl-octyl)-amide | | |
| 93. | 2-Methoxy-quinoline-3-carboxylic acid | 175-181 | 478 |
| | 1(S)-benzyl-2(S)-hydroxy-6-methyl- | | |
| | 4(R)-methylcarbamoyl-heptyl)-amide, | | |
| 94. | 5,6-Dichloro-1H-benzoimidazole-2- | 114-117 | 505 |
| | carboxylic acid 1(S)-benzyl-2(S)- | | |
| | hydroxy-6-methyl-4(R)- | | |
| | methylcarbamoyl-heptyl)-amide | | |
| 95. | Benzothiazole-2-carboxylic acid | 86-89 | 454 |
| | 1(S)-benzyl-2(S)-hydroxy-6-methyl- | | |
| | 4(R)-methylcarbamoyl-heptyl)-amide | | |
| 96. | 7,8-Difluoro-quinoline-3-carboxylic acid | 179-182 | 484 |
| | 1(S)-benzyl-2(S)-hydroxy-6-methyl- | | |
| | 4(R)-methylcarbamoyl-heptyl)-amide | | |
| 97. | 6,7,8-Trifluoro-quinoline-3-carboxylic | 156-161 | 502, |
| | acid | | 484 |
| | 1(S)-benzyl-2(S)-hydroxy-6-methyl- | | |
| | 4(R)-methylcarbamoyl-heptyl)-amide | | |
| 98. | 5,8-Dimethyl-quinoline-3-carboxylic | 197-199 | 476 |
| | acid 1(S)-benzyl-2(S)-hydroxy-6- | | |
| | methyl-4(R)-methylcarbamoyl-heptyl)- | | |
| | amide | | |
| 99. | Quinoxaline-2-carboxylic acid | 103-106 | 505 |
| | 1(S)-benzyl-4(R)-butylcarbamoyl-2(S)- | | |
| | hydroxy-7-methyl-octyl)-amide | | |
| 100. | Quinoline-3-carboxylic acid | | 516 |
| | [1(S)-(3,4-dichloro-benzyl)-2(S)- | | |
| | hydroxy-6-methyl-4(R)- | | |
| | methylcarbamoyl-heptyl]-amide | | |
| 101. | 5,6,7,8-Tetrahydro-quinoline-3- | 169.5-172.5 | 466 |
| | carboxylic acid | | |
| | 1(S)-benzyl-2(S)-hydroxy-7-methyl- | | |
| | 4(R)-methylcarbamoyl-octyl)-amide | | |
| 102. | Quinoline-3-carboxylic acid | 176-178 | 474 |
| | 1(S)-benzyl-5-cyclopentyl-2(S)- | | |
| | hydroxy-4(R)-methylcarbamoyl-pentyl)- | | |
| | amide | | |
| 103. | Quinoxaline-2-carboxylic acid | 120-122 | 475 |
| | 1(S)-benzyl-5-cyclopentyl-2(S)- | | |
| | hydroxy-4(R)-methylcarbamoyl-pentyl)- | | |
| | amide | | |
| 104. | N-1(S)-Benzyl-5-cyclopentyl-2(S)- | 194-198 | 504 |
| | hydroxy-4(R)-methylcarbamoyl-pentyl)- | | |
| | 5-bromo-nicotinamide | | |
| 105. | 5,6,7,8-Tetrahydro-quinoline-3- | 143-146 | 478 |
| | carboxylic acid 1(S)-benzyl-5- | | |
| | cyclopentyl-2(S)-hydroxy-4(R)- | | |
| | methylcarbamoyl-pentyl)-amide, | | |
| 106. | Quinoxaline-2-carboxylic acid | 217-219 | 461, |
| | 1(S)-benzyl-4(R)-carbamoyl-5- | | 444 |
| | cyclopentyl-2(S)-hydroxy-pentyl)-amide | | |
| 107. | 6,7-Dihydro-5H-[1]pyrindine-3- | 154.5-156 | 452, |
| | carboxylic acid | | 349 |
| | 1(S)-benzyl-2(S)-hydroxy-7-methyl- | | |
| | 4(R)-methylcarbamoyl-octyl)-amide | | |
| 108. | Quinoxaline-2-carboxylic acid | 95-98 | 491, |
| | [1(S)-(4,4-difluoro-cyclohexylmethyl)- | | 473 |
| | 2(S)-hydroxy-6-methyl-4(R)- | | |
| | methylcarbamoyl-heptyl]-amide | | |
| 109. | Quinoxaline-2-carboxylic acid | 95-98 | 506, |
| | [1(S)-(4,4-difluoro-cyclohexylmethyl)- | | 488 |
| | 2(S)-hydroxy-7-methyl-4(R)- | | |
| | methylcarbamoyl-octyl]-amide | | |
| 110. | Quinoxaline-2-carboxylic acid | 129-133 | 478 |
| | 1(S)-benzyl-4(R)-ethylcarbamoyl-2(S)- | | |
| | hydroxy-7-methyl-octyl)-amide | | |
| 111. | Quinoxaline-2-carboxylic acid | 125-130 | 492 |
| | 1(S)-benzyl-2(S)-hydroxy-7-methyl- | | |
| | 4(R)-propylcarbamoyl-octyl)-amide | | |
| 112. | Quinoxaline-2-carboxylic acid | 168-169 | 490, |
| | 1(S)-benzyl-4(R)- | | 472 |
| | cyclopropylcarbamoyl-2(S)-hydroxy-7- | | |
| | methyl-octyl)-amide | | |
| 113. | Quinoxaline-2-carboxylic acid | 148-150 | 504, |
| | 1(S)-benzyl-4(R)-cyclobutylcarbamoyl- | | 486 |
| | 2(S)-hydroxy-7-methyl-octyl)-amide | | |
| 114. | Quinoxaline-2-carboxylic acid | 151-154 | 530 |
| | [1(S)-(4-difluoromethoxy-benzyl)-2(S)- | | |
| | hydroxy-7-methyl-4(R)- | | |
| | methylcarbamoyl-octyl]-amide | | |
| 115. | 4-{3(S)-Hydroxy-7-methyl-5(R)- | 87-95 | 508 |
| | methylcarbamoyl-2(S)-[(quinoxaline- | | |
| | 2-carbonyl)-amino]-octyl}-benzoic acid | | |
| | methyl ester | | |
| 116. | Quinoxaline-2-carboxylic acid 1(S)- | | 379 |
| | benzyl-4-carbamoyl-2(S)-hydroxy- | | |
| | butyl)-amide | | |
| 117. | 6,7,8-Trifluoro-quinoline-3-carboxylic | 206-207 | 516, |
| | acid | | 498 |
| | 1(S)-benzyl-2(S)-hydroxy-7-methyl- | | |
| | 4(R)-methylcarbamoyl-octyl)-amide | | |
| 118. | 6,7,8-Trifluoro-quinoline-3-carboxylic | 205-206 | 502, |
| | acid | | 485 |
| | 1(S)-benzyl-4(R)-carbamoyl-2(S)- | | |
| | hydroxy-7-methyl-octyl)-amide | | |
| 119. | 6,8-Difluoro-quinoline-3-carboxylic acid | 198-200 | 498 |
| | 1(S)-benzyl-2(S)-hydroxy-7-methyl- | | |
| | 4(R)-methylcarbamoyl-octyl)-amide | | |
| 120. | 6,8-Difluoro-quinoline-3-carboxylic acid | 188-190 | 484, |
| | 1(S)-benzyl-4(R)-carbamoyl-2(S)- | | 467 |
| | hydroxy-7-methyl-octyl)-amide | | |
| 121. | Quinoxaline-2-carboxylic acid | 102-104 | 517, |
| | 1(S)-benzyl-4(R)-butylcarbamoyl-5- | | 499 |
| | cyclopentyl-2(S)-hydroxy-pentyl)-amide | | |
| 122. | 6-Methyl-pyridine-2-carboxylic acid | 74-76 | |
| | 1(S)-benzyl-2(S)-hydroxy-6-methyl- | | |
| | 4(R)-methylcarbamoyl-heptyl)-amide | | |
| 123. | Quinoxaline-2-carboxylic acid | 145.5-146.5 | 477 |
| | 1(S)-benzyl-2(S)-hydroxy-8-methyl- | | |
| | 4(R)-methylcarbamoyl-nonyl)-amide | | |
| 124. | Quinoxaline-2-carboxylic acid | 163-165 | 463 |
| | 1(S)-benzyl-4(R)-carbamoyl-2(S)- | | |
| | hydroxy-8-methyl-nonyl)-amide | | |
| 125. | Quinoxaline-2-carboxylic acid | 123-125 | 539, |
| | 1(S)-biphenyl-4-ylmethyl-2(S)-hydroxy- | | 521, |
| | 7-methyl-4(R)-methylcarbamoyl-octyl)- | | 508 |
| | amide | | |
| 126. | Quinoxaline-2-carboxylic acid | 168-170 | 447, |
| | 1(S)-benzyl-4(R)-carbamoyl-2(S)- | | 430 |
| | hydroxy-7-methyl-oct-6-enyl)-amide | | |
| 127. | Quinoxaline-2-carboxylic acid | 121-123 | |
| | (2(S)-hydroxy-6-methyl-4(R)- | | |
| | methylcarbamoyl-1(S)-naphthalen-2- | | |
| | ylmethyl-heptyl)-amide | | |
| 128. | Quinoxaline-2-carboxylic acid | 77-79 | 463, |
| | 1(S)-benzyl-4(R)-carbamoyl-2(S)- | | 446 |
| | hydroxy-7,7-dimethyl-octyl)-amide | | |
| 129. | Quinoxaline-2-carboxylic acid | 195-199 | 477, |
| | 1(S)-benzyl-2(S)-hydroxy-7,7-dimethyl- | | 459 |
| | 4(R)-methylcarbamoyl-octyl)-amide | | |
| 130. | Quinoxaline-2-carboxylic acid | 168-172 | 469, |
| | 1(S)-benzyl-4(R)-carbamoyl-2(S)- | | 452 |
| | hydroxy-5-phenyl-pentyl)-amide | | |
| 131. | Quinoxaline-2-carboxylic acid | 205-206 | 508 |
| | 1(S)-biphenyl-4-ylmethyl-4(R)- | | |
| | carbamoyl-2(S)-hydroxy-7-methyl- | | |
| | octyl)-amide | | |
| 132. | Quinoxaline-2-carboxylic acid | 170-172 | 525, |
| | [1(S)-benzyl-5-(4,4-difluoro- | | 507 |
| | cyclohexyl)-2(S)-hydroxy-4(R)- | | |
| | methylcarbamoyl-pentyl]-amide | | |
| 133. | Quinoxaline-2-carboxylic acid | 174-176 | 511, |
| | [1(S)-benzyl-4(R)-carbamoyl-5-(4,4- | | 493 |
| | difluoro-cyclohexyl)-2(S)-hydroxy- | | |
| | pentyl]-amide | | |
| 134. | Quinoxaline-2-carboxylic acid | 158.5-159.5 | 481, |
| | [1(S)-(3-fluoro-benzyl)-2(S)-hydroxy-7- | | 463 |
| | methyl-4(R)-methylcarbamoyl-octyl]- | | |
| | amide | | |
| 135. | Quinoxaline-2-carboxylic acid | 191-191.5 | 467, |
| | [4(R)-carbamoyl-1(S)-(3-fluoro-benzyl)- | | 449 |
| | 2(S)-hydroxy-7-methyl-octyl]-amide | | |
| 136. | Quinoxaline-2-carboxylic acid | 65-68 | 461, |
| | 1(S)-benzyl-2(S)-hydroxy-7-methyl- | | 443 |
| | 4(R)-methylcarbamoyl-oct-6-enyl)- | | |
| | amide | | |
| 137. | 6,7,8-Trifluoro-quinoline-3-carboxylic | 158-161 | 541, |
| | acid 1(S)-benzyl-2(S)-hydroxy-7(S)- | | 523 |
| | methyl-4(R)-methylcarbamoyl-nonyl)- | | |
| | amide | | |
| 138. | Quinoxaline-2-carboxylic acid | 185-187 | 446 |
| | 1(S)-benzyl-4(R)-carbamoyl-2(S)- | | |
| | hydroxy-7(S)-methyl-nonyl)-amide | | |
| 139. | Quinoxaline-2-carboxylic acid | 148-150 | 482, |
| | 1(S)-benzyl-7-fluoro-2(S)-hydroxy-7- | | 463 |
| | methyl-4(R)-methylcarbamoyl-octyl)- | | |
| | amide | | |
| 140. | Quinoxaline-2-carboxylic acid | 184-186 | 467, |
| | 1(S)-benzyl-4(R)-carbamoyl-7-fluoro- | | 449 |
| | 2(S)-hydroxy-7-methyl-octyl)-amide | | |
| 141. | Quinoxaline-2-carboxylic acid | 137-139.5 | 478 |
| | 1(S)-benzyl-2(S)-hydroxy-7-methyl- | | |
| | 4(R)-methylcarbamoyl-nonyl)-amide | | |
| 142. | Quinoxaline-2-carboxylic acid | 68-70 | |
| | 1(S)-benzyl-4(R)-dimethylcarbamoyl- | | |
| | 2(S)-hydroxy-7-methyl-octyl)-amide | | |
| 143. | 7,8-Difluoro-quinoline-3-carboxylic acid | 175 | 518, |
| | 1(S)-benzyl-2(S)-hydroxy-4(R)- | (Dec.) | 500 |
| | methylcarbamoyl-5-phenyl-pentyl)- | | |
| | amide | | |
| 144. | 7,8-Difluoro-quinoline-3-carboxylic acid | 198-201 | 498, |
| | 1(S)-benzyl-2(S)-hydroxy-7-methyl- | | 480 |
| | 4(R)-methylcarbamoyl-octyl)-amide | | |
| 145. | 8-Fluoro-quinoline-3-carboxylic acid | 179-183 | 480, |
| | 1(S)-benzyl-2(S)-hydroxy-7-methyl- | | 462 |
| | 4(R)-methylcarbamoyl-octyl)-amide | | |
| 146. | Quinoxaline-2-carboxylic acid | 130-132 | 462, |
| | 1(S)-benzyl-2(S)-hydroxy-4(R)- | | 448 |
| | methylcarbamoyl-non-6-enyl)-amide | | |
| 147. | Quinoxaline-2-carboxylic acid | 154-155 | 448, |
| | 1(S)-benzyl-4(R)-carbamoyl-2(S)- | | 430 |
| | hydroxy-non-6-enyl)-amide | | |
| 148. | 7,8-Difluoro-quinoline-3-carboxylic acid | 188-190 | 485, |
| | 1(S)-benzyl-4(R)-carbamoyl-2(S)- | | 467 |
| | hydroxy-7-methyl-octyl)-amide | | |
| 149. | 8-Fluoro-quinoline-3-carboxylic acid | 192-196 | 466, |
| | 1(S)-benzyl-4(R)-carbamoyl-2(S)- | | 449 |
| | hydroxy-7-methyl-octyl)-amide | | |
| 150. | Quinoxaline-2-carboxylic acid | 188.5-189.5 | 450 |
| | 1(S)-benzyl-4(R)-carbamoyl-2(S)- | | |
| | hydroxy-nonyl)-amide | | |
| 151. | 2(S)-{2(S)-hydroxy-4-phenyl-3(S)- | 178-180 | |
| | [(quinoxaline-2-carbonyl)-amino]-butyl}- | | |
| | N1,N4-dimethyl-succinamide | | |
| 152. | Quinoxaline-2-carboxylic acid | 105-108 | 496 |
| | 1(S)-benzyl-4(R)-ethylcarbamoyl-7- | | |
| | fluoro-2(S)-hydroxy-7-methyl-octyl)- | | |
| | amide | | |
| 153. | Quinoxaline-2-carboxylic acid | 110-112 | 523, |
| | 1(S)-benzyl-4(R)-butylcarbamoyl-7- | | 505 |
| | fluoro-2(S)-hydroxy-7-methyl-octyl)- | | |
| | amide | | |
| 154. | Quinoxaline-2-carboxylic acid | 145-147 | 499 |
| | [7-fluoro-1(S)-(4-fluoro-benzyl)-2(S)- | | |
| | hydroxy-7-methyl-4(R)- | | |
| | methylcarbamoyl-octyl]-amide | | |
| 155. | Quinoxaline-2-carboxylic acid | 206-207 | 536, |
| | [4(R)-carbamoyl-1(S)-(3,4-dichloro- | | 518 |
| | benzyl)-7-fluoro-2(S)-hydroxy-7-methyl- | | |
| | octyl]-amide | | |
| 156. | 7,8-Difluoro-quinoline-3-carboxylic acid | 187-189 | 571 |
| | [4(R)-carbamoyl-1(S)-(3,4-dichloro- | | |
| | benzyl)-7-fluoro-2(S)-hydroxy-7-methyl- | | |
| | octyl]-amide | | |
| 157. | Quinoxaline-2-carboxylic acid | 223-225 | 478 |
| | (4(R)-carbamoyl-2(S)-hydroxy-7- | | |
| | methyl-1(S)-phenethyl-octyl)-amide, | | |
| 158. | 7,8-Difluoro-quinoline-3-carboxylic acid | 208-210 | 463, |
| | [4(R)-carbamoyl-7-fluoro-1(S)-(4- | | 445 |
| | fluoro-benzyl)-2(S)-hydroxy-7-methyl- | | |
| | octyl]-amide | | |
| 159. | Quinoxaline-2-carboxylic acid | | 520 |
| | [4(R)-carbamoyl-7-fluoro-1(S)-(4- | | |
| | fluoro-benzyl)-2(S)-hydroxy-7-methyl- | | |
| | octyl]-amide | | |
| 160. | Quinoxaline-2-carboxylic acid | | 551 |
| | [1(S)-benzyl-7-fluoro-2(S)-hydroxy-7- | | |
| | methyl-4(R)-(4-methyl-piperazine-1- | | |
| | carbonyl)-octyl]-amide, | | |
| 161. | Quinoxaline-2-carboxylic acid | 212-214 | 477, |
| | [1(S)-benzyl-4(R)-carbamoyl-2(S)- | | 459 |
| | hydroxy-5-(tetrahydro-pyran-4(R)-yl)- | | |
| | pentyl]-amide | | |
| 162. | Quinoxaline-2-carboxylic acid | | 536 |
| | [1(S)-benzyl-7-fluoro-2(S)-hydroxy-7- | | |
| | methyl-4(R)-(piperidine-1-carbonyl)- | | |
| | octyl]-amide | | |
| 163. | Quinoxaline-2-carboxylic acid | | 537 |
| | [1(S)-benzyl-7-fluoro-2(S)-hydroxy-7- | | |
| | methyl-4(R)-(morpholine-4-carbonyl)- | | |
| | octyl]-amide, | | |
| 164. | Quinoxaline-2-carboxylic acid | 90-100 | 481, |
| | [1(S)-benzyl-3-(2-carbamoyl-indan-2- | | 464 |
| | yl)-2(S)-hydroxy-propyl]-amide | | |
| 165. | Quinoxaline-2-carboxylic acid | 212-216 | |
| | 1(S)-benzyl-2(S)-hydroxy-4(R)- | (Dec.) | |
| | methylcarbamoyl-7-phenyl-hept-6- | | |
| | enyl)-amide | | |
| 166. | Quinoline-2-carboxylic acid | 163.5-165 | 466, |
| | 1(S)-benzyl-4(R)-carbamoyl-7-fluoro- | | 449 |
| | 2(S)-hydroxy-7-methyl-octyl)-amide | | |
| 167. | 6,7-Dihydro-5H-[1]pyrindine-3- | 175-178 | 456 |
| | carboxylic acid | | |
| | 1(S)-benzyl-4(R)-carbamoyl-7-fluoro- | | |
| | 2(S)-hydroxy-7-methyl-octyl)-amide | | |
| 168. | Quinoxaline-2-carboxylic acid (1(S)- | 222-223 | 461, |
| | benzyl-4-carbamoyl-4(S)-cyclohexyl- | | 444 |
| | 2(S)-hydroxy-butyl)-amide; | | |
| 169. | Quinoxaline-2-carboxylic acid (1(S)- | 178-180 | 461, |
| | benzyl-4-carbamoyl-4(S)-cyclohexyl- | | 444 |
| | 2(S)-hydroxy-butyl)-amide | | |
| 170. | Quinoxaline-2-carboxylic acid (1(S)- | 229-232 | 447 |
| | benzyl-4-carbamoyl-4(S)-cyclohexyl- | | |
| | 2(S)-hydroxy-butyl)-amide | | |
| 171. | Quinoxaline-2-carboxylic acid (1(S)- | 126-128 | 447 |
| | benzyl-4-carbamoyl-4(S)-cyclopentyl- | | |
| | 2(S)-hydroxy-butyl)-amide; | | |
| 172. | Quinoline-3-carboxylic acid | 200-202 | 466, |
| | 1(S)-benzyl-4(R)-carbamoyl-7-fluoro- | | 449 |
| | 2(S)-hydroxy-7-methyl-octyl)-amide | | |
| 173. | N-1(S)-Benzyl-4(R)-carbamoyl-7- | 181-183 | 476 |
| | fluoro-2(S)-hydroxy-7-methyl-octyl)-5- | | |
| | bromo-nicotinamide | | |
| 174. | Quinoxaline-2-carboxylic acid | 184-187 | 466, |
| | [4(R)-carbamoyl-1-(2(S)-fluoro-benzyl)- | | 448 |
| | 2(S)-hydroxy-7-methyl-octyl]-amide | | |
| 175. | Quinoxaline-2-carboxylic acid | 213-215 | 466 |
| | [4(R)-carbamoyl-1(S)-(2-fluoro-benzyl)- | | |
| | 2(S)-hydroxy-7-methyl-octyl]-amide | | |
| 176. | Quinoxaline-2-carboxylic acid [1(S)- | | 502 |
| | benzyl-4(S)-carbamoyl-2(S)-hydroxy-4- | | |
| | (4-isopropyl-cyclohexyl)-butyl]-amide; | | |
| 177. | Quinoxaline-2-carboxylic acid | | 454, |
| | (4(R)-carbamoyl-2(S)-hydroxy-7- | | 436 |
| | methyl-1(S)-thiophen-2-ylmethyl-octyl)- | | |
| | amide | | |
| 178. | Quinoxaline-2-carboxylic acid | 195-196 | 456 |
| | (4(R)-carbamoyl-2(S)-hydroxy-7- | | |
| | methyl-1(S)-thiazol-4-ylmethyl-octyl)- | | |
| | amide | | |
| 179. | Quinoxaline-2-carboxylic acid [1(S)- | 188-190 | 516 |
| | benzyl-4(S)-carbamoyl-2(S)-hydroxy-4- | | |
| | (3,3,5,5-tetramethyl-cyclohexyl)-butyl]- | | |
| | amide | | |
| 180. | Quinoxaline-2-carboxylic acid (1(S)- | | 495 |
| | benzyl-4(S)-carbamoyl-2(S)-hydroxy-4- | | |
| | indan-2-yl-butyl)-amide; | | |
| 181. | Quinoxaline-2-carboxylic acid (1(S)- | 216-217 | 474, |
| | benzyl-4(S)-carbamoyl-4-cycloheptyl- | | 457 |
| | 2(S)-hydroxy-butyl)-amide; | | |
| 182. | Quinoxaline-2-carboxylic acid (1(S)- | | 477 |
| | benzyl-4(S)-carbamoyl-2(S)-hydroxy-5- | | |
| | propyl-octyl)-amide; | | |
| 183. | Quinoxaline-2-carboxylic acid (1(S)- | | |
| | benzyl-4(S)-carbamoyl-2(S)-hydroxy-5- | | |
| | propyl-oct-5-enyl)-amide; | | |
| 184. | Quinoxaline-2-carboxylic acid | | |
| | 1(S)-benzyl-4(R)-carbamoyl-2(S),7- | | |
| | dihydroxy-7-methyl-octyl)-amide | | |
| 185. | Quinoxaline-2-carboxylic acid | | 467, |
| | 1(S)-benzyl-7-chloro-2(S)-hydroxy- | | 449 |
| | 4(R)-methylcarbamoyl-hept-6-enyl)- | | |
| | amide | | |
| 186. | Quinoxaline-2-carboxylic acid | | 467, |
| | 1(S)-benzyl-7-chloro-2(S)-hydroxy- | | 449 |
| | 4(R)-methylcarbamoyl-hept-6-enyl)- | | |
| | amide | | |
| 187. | Quinoxaline-2-carboxylic acid | 160-162 | 467, |
| | 1(S)-benzyl-6-chloro-2(S)-hydroxy- | | 449 |
| | 4(S)-methylcarbamoyl-hept-6-enyl)- | | |
| | amide | | |
| 188. | Quinoxaline-2-carboxylic acid | 203-204.5 | |
| | 1(S)-benzyl-4(R)-carbamoyl-6-chloro- | | |
| | 2(S)-hydroxy-hept-6-enyl)-amide | | |
| 189. | Quinoxaline-2-carboxylic acid | 171-174 | 447, |
| | 1(S)-benzyl-4(S)-carbamoyl-6- | | 429 |
| | cyclopropyl-2(S)-hydroxy-hexyl)-amide | | |
| 190. | Quinoxaline-2-carboxylic acid | 146-148 | 461, |
| | 1(S)-benzyl-6-cyclopropyl-2(S)- | | 443 |
| | hydroxy-4(R)-methylcarbamoyl-hexyl)- | | |
| | amide | | |
| 191. | Quinoxaline-2-carboxylic acid [1(S)- | 218-220 | 475, |
| | benzyl-4(R)-carbamoyl-2(S)-hydroxy- | | 457 |
| | 4(S)-(4-methyl-cyclohexyl)-butyl]- | | |
| | amide; | | |
| 192. | Quinoxaline-2-carboxylic acid (1(S)- | 190-191 | 495, |
| | benzyl-4(S)-carbamoyl-2(S)-hydroxy-4- | | 477 |
| | indan-2-yl-butyl)-amide; | | |
| 193. | Quinoxaline-2-carboxylic acid | 184-187 | 553, |
| | [1(S)-benzyl-4(R)-carbamoyl-2(S)- | | 536 |
| | hydroxy-5-(4-trifluoromethoxy-phenyl)- | | |
| | pentyl]-amide | | |
| 194. | Quinoxaline-2-carboxylic acid | 164-166 | 487, |
| | [1(S)-benzyl-4(R)-carbamoyl-5-(4- | | 470 |
| | fluoro-phenyl)-2(S)-hydroxy-pentyl]- | | |
| | amide | | |
| 195. | Quinoxaline-2-carboxylic acid | 165-166 | 436 |
| | 1(S)-benzyl-4(R)-carbamoyl-7-chloro- | | |
| | 2(S)-hydroxy-hept-6-enyl)-amide | | |
| 196. | Quinoxaline-2-carboxylic acid | 158-160 | 436 |
| | 1(S)-benzyl-4(R)-carbamoyl-7-chloro- | | |
| | 2(S)-hydroxy-hept-6-enyl)-amide | | |
| 197. | 3-Hydroxy-quinoxaline-2-carboxylic | 185-189 | 483, |
| | acid 1(S)-benzyl-4(R)-carbamoyl-7- | | 465 |
| | fluoro-2(S)-hydroxy-7-methyl-octyl)- | | |
| | amide | | |
| 198. | Quinoxaline-2-carboxylic acid | 183-184 | |
| | 1(S)-benzyl-4(R)-benzylcarbamoyl-7- | | |
| | fluoro-2(S)-hydroxy-7-methyl-octyl)- | | |
| | amide | | |
| 199. | Quinoxaline-2-carboxylic acid | 188-191 | |
| | {1(S)-benzyl-7-fluoro-2(S)-hydroxy-7- | | |
| | methyl-4(R)-[(pyridin-3-ylmethyl)- | | |
| | carbamoyl]-octyl}-amide | | |
| 200. | Quinoxaline-2-carboxylic acid | | 571, |
| | 1(S)-benzyl-8,8-trifluoro-2(S)-hydroxy- | | 553 |
| | 4(R)-methylcarbamoyl-7- | | |
| | trifluoromethyl-octyl)-amide | | |
| 201. | Quinoxaline-2-carboxylic acid | 187-193 | 553 |
| | 1(S)-benzyl-4(R)-carbamoyl-8,8- | | |
| | trifluoro-2(S)-hydroxy-7-trifluoromethyl- | | |
| | octyl)-amide | | |
| 202. | Quinoxaline-2-carboxylic acid | 170-173 | 502 |
| | [2(S)-hydroxy-7-methyl-4(R)- | | |
| | methylcarbamoyl-1(S)-(4- | | |
| | methylcarbamoyl-benzyl)-octyl]-amide | | |
| 203. | Quinoxaline-2-carboxylic acid (1(S)- | 215-218 | 448, |
| | benzyl-4(S)-carbamoyl-5-ethyl-2(S)- | | 431 |
| | hydroxy-heptyl)-amide; | | |
| 204. | Quinoxaline-2-carboxylic acid [1(S)- | 151-154 | |
| | benzyl-4(S)-carbamoyl-2(S)-hydroxy-4- | | |
| | (tetrahydro-pyran-4-yl)-butyl]-amide; | | |
| 205. | Quinoxaline-2-carboxylic acid | 155-156 | 572 |
| | [1(S)-benzyl-7-fluoro-2(S)-hydroxy-7- | | |
| | methyl-4(R)-(2-pyridin-2-yl- | | |
| | ethylcarbamoyl)-octyl]-amide | | |
| 206. | Quinoxaline-2-carboxylic acid | 162-164 | 617 |
| | [1(S)-benzyl-4(R)-(3,4-dimethoxy- | | |
| | benzylcarbamoyl)-7-fluoro-2(S)- | | |
| | hydroxy-7-methyl-octyl]-amide | | |
| 207. | Quinoxaline-2-carboxylic acid 1(S)- | | 420 |
| | benzyl-4(R)-carbamoyl-2(S)-hydroxy-6- | | |
| | methoxy-hexyl)-amide | | |
| 208. | Quinoxaline-2-carboxylic acid | 172-175 | 450 |
| | 1(S)-benzyl-4(R)-carbamoyl-7-chloro- | | |
| | 2(S)-hydroxy-oct-6-enyl)-amide | | |
| 209. | Quinoxaline-2-carboxylic acid | 108-111 | 463 |
| | 1(S)-benzyl-7-chloro-2(S)-hydroxy- | | |
| | 4(R)-methylcarbamoyl-oct-6-enyl)- | | |
| | amide | | |
| 210. | Quinoxaline-2-carboxylic acid [1(S)- | 221-222 | 489, |
| | benzyl-4(R)-carbamoyl-4-(3,5-dimethyl- | | 471 |
| | cyclohexyl)-2(S)-hydroxy-butyl]-amide; | | |
| 211. | Quinoxaline-2-carboxylic acid {1(S)- | 138-140 | 557, |
| | benzyl-7-fluoro-2(S)-hydroxy-7-methyl- | | 540 |
| | 4(R)-[(pyridin-2-ylmethyl)-carbamoyl]- | | |
| | octyl}-amide | | |
| 212. | Quinoxaline-2-carboxylic acid {1(S)- | 138-140 | 587, |
| | benzyl-7-fluoro-2(S)-hydroxy-4(R)-[2- | | 569 |
| | (4-hydroxy-phenyl)-ethylcarbamoyl]-7- | | |
| | methyl-octyl}-amide | | |
| 213. | Quinoxaline-2-carboxylic acid {1(S)- | 174-175 | 563, |
| | benzyl-7-fluoro-2(S)-hydroxy-7-methyl- | | 545 |
| | 4(R)-[(thiophen-2-ylmethyl)- | | |
| | carbamoyl]-octyl}-amide | | |
| 214. | Quinoxaline-2-carboxylic acid | 194.5-196.5 | 482 |
| | 1(S)-benzyl-4(R)-carbamoyl-2(S)- | | |
| | hydroxy-6-phenoxy-hexyl)-amide | | |
| 215. | Quinoxaline-2-carboxylic acid | 113-118 | 448 |
| | 1(S)-benzyl-4(R)-carbamoyl-2(S)- | (Mix) | |
| | hydroxy-6-isopropoxy-hexyl)-amide | | |
| 216. | Quinoxaline-2-carboxylic acid {1(S)- | 207-210 | 650 |
| | benzyl-7-fluoro-2(S)-hydroxy-7-methyl- | | |
| | 4(R)-[2-(4-sulfamoyl-phenyl)- | | |
| | ethylcarbamoyl]-octyl}-amide | | |
| 217. | Quinoxaline-2-carboxylic acid {1(S)- | 100-104 | 558 |
| | benzyl-7-fluoro-2(S)-hydroxy-7-methyl- | | |
| | 4(R)-[(pyridin-4-ylmethyl)-carbamoyl]- | | |
| | octyl}-amide | | |
| 218. | Quinoxaline-2-carboxylic acid [1(S)- | 78-79 | 555, |
| | benzyl-4(R)-(2-ethylsulfanyl- | | 537 |
| | ethylcarbamoyl)-7-fluoro-2(S)-hydroxy- | | |
| | 7-methyl-octyl]-amide | | |
| 219. | Quinoxaline-2-carboxylic acid [1(S)- | 48-50 | 507 |
| | benzyl-7-fluoro-2(S)-hydroxy-4(R)-(2- | | |
| | methoxy-ethylcarbamoyl)-7-methyl- | | |
| | octyl]-amide | | |
| 220. | Quinoxaline-2-carboxylic acid [1(S)- | 154-155 | 572 |
| | benzyl-7-fluoro-2(S)-hydroxy-7-methyl- | | |
| | 4(R)-(2-pyridin-3-yl-ethylcarbamoyl)- | | |
| | octyl]-amide | | |
| 221. | Quinoxaline-2-carboxylic acid [1(S)- | 78-80 | 572 |
| | benzyl-7-fluoro-2(S)-hydroxy-7-methyl- | | |
| | 4(R)-(2-pyridin-4-yl-ethylcarbamoyl)- | | |
| | octyl]-amide | | |
| 222. | Quinoxaline-6-carboxylic acid | 190-192 | 467 |
| | 1(S)-benzyl-4(R)-carbamoyl-7-fluoro- | | |
| | 2(S)-hydroxy-7-methyl-octyl)-amide | | |
| 223. | Quinoxaline-2-carboxylic acid | 184-189 | 479, |
| | 1(S)-benzyl-6-tert-butoxy-4(R)- | | 461 |
| | carbamoyl-2(S)-hydroxy-hexyl)-amide | | |
| 224. | Quinoxaline-2-carboxylic acid {1(S)- | 100-105 | 574 |
| | benzyl-7-fluoro-2(S)-hydroxy-7-methyl- | | |
| | 4(R)-[2-1-methyl-1H-pyrrol-2-yl)- | | |
| | ethylcarbamoyl]-octyl}-amide | | |
| 225. | Quinoxaline-2-carboxylic acid [1(S)- | 140-150 | 511, |
| | benzyl-4(S)-carbamoyl-4-(1,1-dioxo- | | 494 |
| | thiopyran-4-yl)-2(S)-hydroxy-butyl]- | | |
| | amide; | | |
| 226. | Quinoxaline-2-carboxylic acid {1(S)- | | 640, |
| | benzyl-7-fluoro-2(S)-hydroxy-4(R)-[2- | | 622 |
| | (6-methoxy-1H-indol-3-yl)- | | |
| | ethylcarbamoyl]-7-methyl-octyl}-amide, | | |
| 227. | Quinoxaline-2-carboxylic acid | 135 | 587, |
| | [1(S)-benzyl-7-fluoro-2(S)-hydroxy- | | 569 |
| | 4(R)-(2-methoxy-benzylcarbamoyl)-7- | | |
| | methyl-octyl]-amide | | |
| 228. | Quinoxaline-2-carboxylic acid | | 587, |
| | [1(S)-benzyl-7-fluoro-2(S)-hydroxy- | | 569 |
| | 4(R)-(3-methoxy-benzylcarbamoyl)-7- | | |
| | methyl-octyl]-amide | | |
| 229. | Quinoxaline-2-carboxylic acid [1(S)- | 152-154 | 577 |
| | benzyl-7-fluoro-2(S)-hydroxy-7-methyl- | | |
| | 4(R)-(2-thiophen-2-yl-ethylcarbamoyl)- | | |
| | octyl]-amide | | |
| 230. | Quinoxaline-2-carboxylic acid {1(S)- | 107-108 | 610 |
| | benzyl-7-fluoro-2(S)-hydroxy-4(R)-[2- | | |
| | (1H-indol-3-yl)-ethylcarbamoyl]-7- | | |
| | methyl-octyl}-amide | | |
| 231. | Quinoxaline-2-carboxylic acid {4(R)-[2- | | 586 |
| | (4-amino-phenyl)-ethylcarbamoyl]-1(S)- | | |
| | benzyl-7-fluoro-2(S)-hydroxy-7-methyl- | | |
| | octyl}-amide | | |
| 232. | Quinoxaline-2-carboxylic acid {1(S)- | 109-112 | 631, |
| | benzyl-4(R)-[2-(3,5-dimethoxy-phenyl)- | | 613 |
| | ethylcarbamoyl]-7-fluoro-2(S)-hydroxy- | | |
| | 7-methyl-octyl}-amide | | |
| 233. | Quinoxaline-2-carboxylic acid {1(S)- | | 631, |
| | benzyl-4(R)-[2-(3,4-dimethoxy-phenyl)- | | 613 |
| | ethylcarbamoyl]-7-fluoro-2(S)-hydroxy- | | |
| | 7-methyl-octyl}-amide | | |
| 234. | Quinoxaline-2-carboxylic acid {1(S)- | 155.5-156.5 | 547 |
| | benzyl-7-fluoro-4(R)-[(furan-2- | | |
| | ylmethyl)-carbamoyl]-2(S)-hydroxy-7- | | |
| | methyl-octyl}-amide | | |
| 235. | Quinoxaline-2-carboxylic acid {1(S)- | | 631, |
| | benzyl-4(R)-[2-(2,5-dimethoxy-phenyl)- | | 613 |
| | ethylcarbamoyl]-7-fluoro-2(S)-hydroxy- | | |
| | 7-methyl-octyl}-amide | | |
| 236. | Quinoxaline-2-carboxylic acid | 114-115 | 587, |
| | [1(S)-benzyl-7-fluoro-2(S)-hydroxy- | | 569 |
| | 4(R)-(4-methoxy-benzylcarbamoyl)-7- | | |
| | methyl-octyl]-amide | | |
| 237. | Quinoxaline-2-carboxylic acid | 150-152 | 505, |
| | 1(S)-benzyl-4(R)-carbamoyl-6- | | 487 |
| | cyclohexyloxy-2(S)-hydroxy-hexyl)- | | |
| | amide | | |
| 238. | Quinoxaline-2-carboxylic acid {4(R)- | | 596 |
| | [(1H-benzoimidazol-2-ylmethyl)- | | |
| | carbamoyl]-1(S)-benzyl-7-fluoro-2(S)- | | |
| | hydroxy-7-methyl-octyl}-amide | | |
| 239. | Quinoxaline-2-carboxylic acid [1(S)- | 217-219 | 551, |
| | benzyl-7-fluoro-2(S)-hydroxy-4(R)- | | 533 |
| | (2(S)-hydroxymethyl-pyrrolidine-1- | | |
| | carbonyl)-7-methyl-octyl]-amide | | |
| 240. | Quinoxaline-2-carboxylic acid {1(S)- | 111-115 | 551, |
| | benzyl-7-fluoro-2(S)-hydroxy-7-methyl- | | 533 |
| | 4(R)-[(tetrahydrofuran-2-ylmethyl)- | | |
| | carbamoyl]-octyl}-amide | | |
| 241. | Quinoxaline-2-carboxylic acid [1(S)- | 176-179 | 497, |
| | benzyl-4(S)-carbamoyl-4-(4,4-difluoro- | | 478 |
| | cyclohexyl)-2(S)-hydroxy-butyl]-amide | | |
| 242. | Quinoxaline-2-carboxylic acid | 99-101 | |
| | [1(S)-benzyl-4(R)-(2,3-dimethoxy- | | |
| | benzylcarbamoyl)-7-fluoro-2(S)- | | |
| | hydroxy-7-methyl-octyl]-amide | | |
| 243. | Quinoxaline-2-carboxylic acid [1(S)- | 187-189 | 477, |
| | benzyl-4(S)-carbamoyl-2(S)-hydroxy-4- | | 379 |
| | (1-hydroxy-cyclohexyl)-butyl]-amide; | | |
| 244. | Quinoxaline-2-carboxylic acid [1(S)- | 195-198 | 491 |
| | benzyl-4(S)-carbamoyl-4-(2,6-dimethyl- | | |
| | tetrahydro-pyran-4-yl)-2(S)-hydroxy- | | |
| | butyl]-amide; | | |
| 245. | Quinoxaline-2-carboxylic acid | 225-227 | 485, |
| | [4(R)-carbamoyl-7-fluoro-1(S)-(3- | | 467 |
| | fluoro-benzyl)-2(S)-hydroxy-7-methyl- | | |
| | octyl]-amide | | |
| 246. | 7,8-Difluoro-quinoline-3-carboxylic acid | >220 | 502, |
| | 1(S)-benzyl-4(R)-carbamoyl-7-fluoro- | | 485 |
| | 2(S)-hydroxy-7-methyl-octyl)-amide | | |
| 247. | N-1(S)-Benzyl-4(R)-carbamoyl-7- | >220 | 484, |
| | fluoro-2(S)-hydroxy-7-methyl-octyl)- | | 466 |
| | 5,6-dichloro-nicotinamide | | |
| 248. | Benzofuran-2-carboxylic acid 1(S)- | 190-192 | 455, |
| | benzyl-4(R)-carbamoyl-7-fluoro-2(S)- | | 438 |
| | hydroxy-7-methyl-octyl)-amide | | |
| 249. | Cinnoline-4-carboxylic acid 1(S)- | 198-199.5 | 469, |
| | benzyl-4(R)-carbamoyl-7-fluoro-2(S)- | | 451 |
| | hydroxy-7-methyl-octyl)-amide | | |
| 250. | Quinoxaline-2-carboxylic acid | 185.5-187.5 | 593, |
| | [4(R)-carbamoyl-7-fluoro-2(S)-hydroxy- | | 576 |
| | 1(S)-(4-iodo-benzyl)-7-methyl-octyl]- | | |
| | amide, | | |
| 251. | Pyrazine-2-carboxylic acid | 211-212 | 417, |
| | 1(S)-benzyl-4(R)-carbamoyl-7-fluoro- | | 319 |
| | 2(S)-hydroxy-7-methyl-octyl)-amide, | | |
| 252. | 6,7,8-Trifluoro-quinoline-3-carboxylic | 195-197 | 520, |
| | acid | | 503 |
| | 1(S)-benzyl-4(R)-carbamoyl-7-fluoro- | | |
| | 2(S)-hydroxy-7-methyl-octyl)-amide, | | |
| 253. | Quinoline-6-carboxylic acid | 170-173 | 466, |
| | 1(S)-benzyl-4(R)-carbamoyl-7-fluoro- | | 449 |
| | 2(S)-hydroxy-7-methyl-octyl)-amide, | | |
| 254. | Isoquinoline-3-carboxylic acid | 194-197 | 466, |
| | 1(S)-benzyl-4(R)-carbamoyl-7-fluoro- | | 448 |
| | 2(S)-hydroxy-7-methyl-octyl)-amide, | | |
| 255. | 2-Methoxy-quinoline-3-carboxylic acid | 213-216 | 496, |
| | 1(S)-benzyl-4(R)-carbamoyl-7-fluoro- | | 479 |
| | 2(S)-hydroxy-7-methyl-octyl)-amide, | | |
| 256. | 1H-Benzoimidazole-2-carboxylic acid | 168-169 | 456, |
| | 1(S)-benzyl-4(R)-carbamoyl-7-fluoro- | | 438 |
| | 2(S)-hydroxy-7-methyl-octyl)-amide, | | |
| 257. | Benzothiazole-2-carboxylic acid | 152.5-155 | 472, |
| | 1(S)-benzyl-4(R)-carbamoyl-7-fluoro- | | 455 |
| | 2(S)-hydroxy-7-methyl-octyl)-amide | | |
| 258. | 5-Methyl-pyrazine-2-carboxylic acid | 194-197 | 431 |
| | 1(S)-benzyl-4(R)-carbamoyl-7-fluoro- | | |
| | 2(S)-hydroxy-7-methyl-octyl)-amide | | |
| 259. | Quinoxaline-2-carboxylic acid | | 470, |
| | 1(S)-benzyl-4(R)-carbamoyl-2(S)- | | 453 |
| | hydroxy-5-pyridin-3-yl-pentyl)-amide | | |
| 260. | Quinoxaline-2-carboxylic acid [1(S)- | 210-211 | 477, |
| | benzyl-4(S)-carbamoyl-2(S)-hydroxy-4- | | 459 |
| | (1-hydroxy-cyclohexyl)-butyl]-amide; | | |
| 261. | Quinoline-3-carboxylic acid (1(S)- | 231 | 460, |
| | benzyl-4(S)-carbamoyl-4-cyclohexyl- | | 443 |
| | 2(S)-hydroxy-butyl)-amide | | |
| 262. | Quinoline-2-carboxylic acid (1(S)- | 208-210 | 460, |
| | benzyl-4(S)-carbamoyl-4-cyclohexyl- | | 443 |
| | 2(S)-hydroxy-butyl)-amide | | |
| 263. | Fluoro-quinoline-3-carboxylic acid | 238-240 | 478, |
| | (1(S)-benzyl-4(S)-carbamoyl-4- | | 461 |
| | cyclohexyl-2(S)-hydroxy-butyl)-amide | | |
| 264. | N-(1(S)-Benzyl-4(S)-carbamoyl-4- | 174-177 | 461 |
| | cyclohexyl-2(S)-hydroxy-butyl)-5,6- | | |
| | dichloro-nicotinamide; | | |
| 265. | N-(1(S)-Benzyl-4(S)-carbamoyl-4- | 255-256 | 475, |
| | cyclohexyl-2(S)-hydroxy-butyl)-5- | | 458 |
| | bromo-nicotinamide; | | |
| 266. | Quinoxaline-2-carboxylic acid | 159-160.5 | 453 |
| | (4(R)-carbamoyl-7-fluoro-2(S)-hydroxy- | | |
| | 7-methyl-1(S)-phenyl-octyl)-amide, | | |
| 267. | Quinoxaline-2-carboxylic acid | | 470, |
| | 1(S)-benzyl-4(R)-carbamoyl-2(S)- | | 453 |
| | hydroxy-5-pyridin-2-yl-pentyl)-amide, | | |
| 268. | Quinoxaline-2-carboxylic acid [4(R)- | 206-207 | 482 |
| | carbamoyl-2(S)-hydroxy-4-(1-hydroxy- | | |
| | cyclohexyl)-1(S)-thiophen-2-ylmethyl- | | |
| | butyl]-amide; | | |
| 269. | Quinoxaline-2-carboxylic acid [1(S)- | 123-125 | 495, |
| | benzyl-4(S)-carbamoyl-2(S)-hydroxy-4- | | 379 |
| | (4-hydroxy-tetrahydro-thiopyran-4-yl)- | | |
| | butyl]-amide; | | |
| 270. | 1,3-Dimethyl-1H-pyrazolo[3,4- | 189.5-191 | 484, |
| | b]pyridine-5-carboxylic acid 1(S)- | | 467 |
| | benzyl-4(R)-carbamoyl-7-fluoro-2(S)- | | |
| | hydroxy-7-methyl-octyl)-amide, | | |
| 271. | Quinoxaline-2-carboxylic acid (1(S)- | 165-166 | |
| | benzyl-7-fluoro-2(S)-hydroxy-4(R)- | | |
| | hydroxycarbamoyl-7-methyl-octyl)- | | |
| | amide | | |
| 272. | Quinoxaline-2-carboxylic acid (1(S)- | | |
| | benzyl-7-fluoro-2(S)-hydroxy-4(R)- | | |
| | methoxycarbamoyl-7-methyl-octyl)- | | |
| | amide | | |
| 273. | 7,8-Difluoro-quinoline-3-carboxylic acid | 233-235 | |
| | (1(S)-benzyl-4(R)-carbamoyl-2(S)- | | |
| | hydroxy-5-phenyl-pentyl)-amide | | |
| 274. | Quinoxaline-2-carboxylic acid [1(S)- | 182-185 | |
| | benzyl-4(R)-carbamoyl-5-(2-chloro- | | |
| | phenyl)-2(S)-hydroxy-pentyl]-amide | | |
| 275. | Quinoxaline-2-carboxylic acid (1(S)- | 168-171 | |
| | benzyl-4(R)-carbamoyl-2(S)-hydroxy-5- | | |
| | o-tolyl-pentyl)-amide | | |
| 276. | Quinoxaline-2-carboxylic acid (1(S)- | 190-192 | |
| | benzyl-2(S)-hydroxy-4(R)- | | |
| | hydroxycarbamoyl-5-phenyl-pentyl)- | | |
| | amide | | |
| 277. | Quinoxaline-2-carboxylic acid [1(S)- | 192-195 | 463, |
| | benzyl-4(S)-carbamoyl-2(S)-hydroxy-4- | | 446 |
| | (1-hydroxy-cyclopentyl)-butyl]-amide | | |
| 278. | Quinoxaline-2-carboxylic acid [1(S)- | 230-233 | 490 |
| | benzyl-4(S)-carbamoyl-2(S)-hydroxy-4- | | |
| | (1-hydroxy-4-methyl-cyclohexyl)-butyl]- | | |
| | amide | | |
| 279. | Quinoxaline-2-carboxylic acid [1(S)- | 199-201 | |
| | benzyl-4(S)-carbamoyl-5-(3,4-dichloro- | | |
| | phenyl)-2(S)-hydroxy-pentyl]-amide | | |
| 280. | Quinoxaline-2-carboxylic acid [1(S)- | 171-173 | |
| | benzyl-4(R)-carbamoyl-5-(2-fluoro- | | |
| | phenyl)-2(S)-hydroxy-pentyl]-amide | | |
| 281. | Quinoxaline-2-carboxylic acid [1(S)- | 110-112 | 477 |
| | benzyl-2(S)-hydroxy-4(S)- | | |
| | hydroxycarbamoyl-4-(1-hydroxy- | | |
| | cyclopentyl)-butyl]-amide | | |
| 282. | Quinoxaline-2-carboxylic acid [1(S)- | 187-188 | 476 |
| | benzyl-4(S)-carbamoyl-2(S)-hydroxy-4- | | |
| | (1-hydroxy-3-methyl-cyclopentyl)-butyl]- | | |
| | amide | | |
| 283. | Quinoxaline-2-carboxylic acid [1(S)- | 114-116 | 506 |
| | benzyl-2(S)-hydroxy-4(S)- | | |
| | hydroxycarbamoyl-4-(1-hydroxy-4- | | |
| | methyl-cyclohexyl)-butyl]-amide | | |
| 284. | N-(1(S)-Benzyl-4(R)-carbamoyl-2(S)- | | 494, |
| | hydroxy-5-phenyl-pentyl)-5-bromo- | | 496 |
| | nicotinamide | | |
| 285. | 8-Fluoro-quinoline-3-carboxylic acid | 206-209 | |
| | (1(S)-benzyl-4(R)-carbamoyl-2(S)- | | |
| | hydroxy-5-phenyl-pentyl)-amide | | |
| 286. | 6,7-Dihydro-5H-[1]pyrindine-3- | 182-186 | |
| | carboxylic acid (1(S)-benzyl-4(R)- | | |
| | carbamoyl-2(S)-hydroxy-5-phenyl- | | |
| | pentyl)-amide | | |
| 287. | Quinoline-3-carboxylic acid (1(S)- | 203-206 | |
| | benzyl-4(R)-carbamoyl-2(S)-hydroxy-5- | | |
| | phenyl-pentyl)-amide | | |
| 288. | Quinoxaline-2-carboxylic acid [1(S)- | 234-236 | 504 |
| | benzyl-4(S)-carbamoyl-2(S)-hydroxy-4- | | |
| | (1-hydroxy-3,5-dimethyl-cyclohexyl)- | | |
| | butyl]-amide | | |
| 289. | Quinoxaline-2-carboxylic acid [1(S)- | | 520 |
| | benzyl-2(S)-hydroxy-4(S)- | | |
| | hydroxycarbamoyl-4-(1-hydroxy-3,5- | | |
| | dimethyl-cyclohexyl)-butyl]-amide | | |
| 290. | Quinoxaline-2-carboxylic acid [1(S)- | 189-191 | 491 |
| | benzyl-4(S)-carbamoyl-2(S)-hydroxy-4- | | |
| | (1-hydroxy-cycloheptyl)-butyl]-amide | | |
| 291. | Quinoxaline-2-carboxylic acid [1(S)- | 118-119 | 506 |
| | benzyl-2(S)-hydroxy-4(S)- | | |
| | hydroxycarbamoyl-4-(1-hydroxy- | | |
| | cycloheptyl)-butyl]-amide | | |
| 292. | Quinoxaline-2-carboxylic acid [1(S)- | 176-179 | |
| | benzyl-4(R)-carbamoyl-5-(3-fluoro- | | |
| | phenyl)-2(S)-hydroxy-pentyl]-amide | | |
| 293. | Quinoxaline-2-carboxylic acid (1(S)- | 178-179 | |
| | benzyl-4(R)-carbamoyl-2(S)-hydroxy-5- | | |
| | m-tolyl-pentyl)-amide | | |
| 294. | Quinoxaline-2-carboxylic acid (1(S)- | 146-148 | |
| | benzyl-2(S)-hydroxy-4- | | |
| | isobutylcarbamoyl-butyl)-amide | | |
| 295. | Quinoxaline-2-carboxylic acid [1(S)- | 206-207 | 528 |
| | benzyl-4(S)-carbamoyl-2(S)-hydroxy-4- | | |
| | (2-hydroxy-adamantan-2-yl)-butyl]- | | |
| | amide | | |
| 296. | Quinoxaline-2-carboxylic acid [1(S)- | 268-269 | 516 |
| | benzyl-4(S)-carbamoyl-2(S)-hydroxy-4- | | |
| | (9-hydroxy-bicyclo[3.3.1]non-9-yl)- | | |
| | butyl]-amide | | |
| 297. | Quinoxaline-2-carboxylic acid [1(S)- | 133-134 | 544 |
| | benzyl-2(S)-hydroxy-4(S)-(2-hydroxy- | | |
| | adamantan-2-yl)-4-hydroxycarbamoyl- | | |
| | butyl]-amide | | |
| 298. | Quinoxaline-2-carboxylic acid [1(S)- | 130-132 | 532 |
| | benzyl-2(S)-hydroxy-4(S)-(9-hydroxy- | | |
| | bicyclo[3.3.1]non-9-yl)-4- | | |
| | hydroxycarbamoyl-butyl]-amide | | |
| 299. | Quinoxaline-2-carboxylic acid [1(S)- | 147-148 | |
| | benzyl-4(R)-carbamoyl-2(S)-hydroxy-5- | | |
| | (3-methoxy-phenyl)-pentyl]-amide | | |
| 300. | Quinoxaline-2-carboxylic acid [1(S)- | 227-228 | 519 |
| | benzyl-4(S)-carbamoyl-2(S)-hydroxy-4- | | |
| | (1-hydroxy-4-propyl-cyclohexyl)-butyl]- | | |
| | amide | | |
| 301. | Quinoxaline-2-carboxylic acid [1(S)- | 115-117 | 533 |
| | benzyl-2(S)-hydroxy-4(S)- | | |
| | hydroxycarbamoyl-4-(1-hydroxy-4- | | |
| | propyl-cyclohexyl)-butyl]-amide | | |
| 302. | Quinoxaline-2-carboxylic acid [1(S)- | | 500, |
| | benzyl-4(R)-carbamoyl-2(S)-hydroxy-5- | | 483 |
| | (4-methoxy-phenyl)-pentyl]-amide | | |
| 303. | Quinoxaline-2-carboxylic acid [1(S)- | 246-248 | 504 |
| | benzyl-4(S)-carbamoyl-4(S)-(4-ethyl-1- | | |
| | hydroxy-cyclohexyl)-2-hydroxy-butyl]- | | |
| | amide | | |
| 304. | Quinoxaline-2-carboxylic acid [1(S)- | 210-211 | 505 |
| | benzyl-4(S)-carbamoyl-2(S)-hydroxy-4- | | |
| | (1-hydroxy-4,4-dimethyl-cyclohexyl)- | | |
| | butyl]-amide | | |
| 305. | Quinoxaline-2-carboxylic acid [1(S)- | 118-123 | 520 |
| | benzyl-2(S)-hydroxy-4(S)- | | |
| | hydroxycarbamoyl-4-(1-hydroxy-4,4- | | |
| | dimethyl-cyclohexyl)-butyl]-amide | | |
| 306. | Quinoxaline-2-carboxylic acid [1(S)- | 207.5-208.5 | |
| | benzyl-4(S)-carbamoyl-4-(4,4-difluoro- | | |
| | 1-hydroxy-cyclohexyl)-2(S)-hydroxy- | | |
| | butyl]-amide | | |
| 307. | Quinoxaline-2-carboxylic acid [1(S)- | 130-131 | 572 |
| | benzyl-4(S)-(4,4-difluoro-1-hydroxy- | | |
| | cyclohexyl)-2(S)-hydroxy-4- | | |
| | hydroxycarbamoyl-but yl]-amide | | |
| 308. | Quinoxaline-2-carboxylic acid [1(S)- | 250-252 | 545 |
| | benzyl-4(S)-carbamoyl-2(S)-hydroxy-4- | | |
| | (1-hydroxy-4-trifluoromethyl- | | |
| | cyclohexyl)-butyl]-amide | | |
| 309. | Quinoxaline-3-carboxylic acid 1(S)- | 94-98 | 454 |
| | cyclohexylmethyl-2(S)-hydroxy-6- | | |
| | methyl-4(R)-methylcarbamoyl-heptyl)- | | |
| | amide | | |
| 310. | Quinoxaline-2-carboxylic acid [1(S)- | 174-175.5 | 522 |
| | benzyl-7-fluoro-2(S)-hydroxy-7-methyl- | | |
| | 4(R)-(pyrrolidine-1-carbonyl)-octyl]- | | |
| | amide | | |
| 311. | N-(1(S)-Benzyl-4(S)-carbamoyl-4- | 218-220 | 470 |
| | cyclohexyl-2(S)-hydroxy-butyl)-5- | | |
| | bromo-nicotinamide | | |
| 312. | Quinoxaline-2-carboxylic acid (1(S)- | 147-149 | 482, 467 |
| | benzyl-7-fluoro-4(R)- | | |
| | hydrazinocarbonyl-2(S)-hydroxyl-7- | | |
| | methyl-octyl)-amide | | |

### Example 313

### Quinoxaline-2-carboxylic Acid (4(r)-carbamoyl-2(s),7-dihydroxy-7-methyl-1(s)-thiophen-2-ylmethyl-octyl)-amide

To a flame dried round bottom flask under a nitrogen atmosphere was added tetrahydrofuran (5 mL) followed by 1,1,1,3,3,3-hexamethyldisilazane (0.78 mL, 3.7 mmol). The mixture was cooled to 0° C. and n-butyl lithium (1.4 mL of a 2.5 M solution in hexanes, 3.38 mmol) was added. The mixture was stirred for 15 minutes, then cooled to −78° C. in dry ice/acetone bath. {1(S)-[5-Oxo-tetrahydro-furan-2(S)-yl]-2-thienyl-ethyl}-carbamic acid tert-butyl ester (500 mg, 1.61 mmol) (prepared by the method of Fray, J. Org. Chem., (51) 4828 (1986) using BOC-L-2-thienylalanine as a starting material) dissolved in tetrahydrofuran (6 mL) was added dropwise via syringe and stirring continued for 30 minutes. A solution of 4-bromo-2-methyl-2-butene (0.21 mL, 1.77 mmol) in 5 mL of THF was added dropwise via syringe. Stirring was continued for 3 hours during which time the temperature rose to −60° C. The mixture was quenched by slow addition of saturated, aqueous ammonium chloride. Upon warming to room temperature, the solution was diluted with ether and transferred to a separatory funnel. The organic phase was washed with saturated aqueous citric acid, saturated aqueous sodium bicarbonate (NaHCO₃), and brine. The organic layer was dried over magnesium sulfate (MgSO₄) and the solvent removed under reduced pressure. Thin layer chromatography in 2:1 hexane/diethyl ether (Et₂O) revealed product with an R_{f} of 0.25. The resulting crude oil was chromatographed on silica gel eluting with 2:1 hexanes/diethyl ether to provide 450 mg (74%) of the lactone.

To the lactone from above (450 mg, 1.19 mmol) was added neat trifluoroacetic acid (4.5 mL). The resulting solution was stirred for 1 hour and the trifluoroacetic acid removed in vacuo. The resulting amine salt (100 mg, 0.34 mmol) was solvated in methylene chloride (15 mL) and triethylamine (0.2 mL, 1.34 mmol). Quinoxalyl chloride (71 mg, 0.37 mmol) was added as a solid and the mixture stirred for 18 hours. The mixture was transferred to a separatory funnel and washed with citric acid, NaHCO₃ and brine. The organic layer was dried (MgSO₄) and the solvents filtered. The filtrate was concentrated in vacuo and the resulting residue was chromatographed on silica gel eluting with 2:1 hexanes:ethyl acetate to provide 108 mg (71%) of the quinoxaline amide. This material was solvated in MeOH and ammonia gas was bubbled in for 5 minutes. The resulting solution was stirred for 16 hour and the solvent removed in vacuo. The remaining residue was recrystallized (methylene chloride/methanol/Hexanes) to provide the title compound (60 mg, 53%). Melting point (MP) 158-159. Low Resolution Mass Spectrum (LRMS) 471, 453, 436. Solubility greater than 250 mg/mL.

Table 2 refers to the preparation of compounds of the formula I by methods analogous to the methods of Example 313.

TABLE 2

| | | M.P. | |
|---|---|---|---|
| Example | Name | (° C.) | LRMS |
| 314. | Quinoxaline-2-carboxylic acid | 161-163 | 499, 481, 464 |
| | 4(R)-carbamoyl-1(S)-(3-chloro- | | |
| | benzyl)-2(S),7-dihydroxy-7- | | |
| | methyl-octyl]-amide | | |
| 315. | 7,8-Difluoro-quinoline-3- | 171-173 | 501, 484 |
| | carboxylic acid (1S)-benzyl- | | |
| | 4(R)-carbamoyl-2(S),7- | | |
| | dihydroxy-7-methyl-octyl)- | | |
| | amide | | |
| 316. | Quinoxaline-2-carboxylic acid | 153-155 | 483, 465, 448 |
| | [4(R)-carbamoyl-1(S)-(3-fluoro- | | |
| | benzyl)-2(S),7-dihydroxy-7- | | |
| | methyl-octyl]-amide | | |
| 317. | 6,7,8-Trifluoro-quinoline-3- | 185-188 | 519, 502 |
| | carboxylic acid (1(S)-benzyl- | | |
| | 4(R)-carbamoyl-2(S),7- | | |
| | dihydroxy-7-methyl-octyl)- | | |
| | amide | | |
| 318. | Quinoxaline-2-carboxylic acid | 108-110 | 482, 464, 447 |
| | (1(S)-benzyl-2(S),7-dihydroxy- | | |
| | 4(R)-hydroxycarbamoyl-7- | | |
| | methyl-octyl)-amide | | |
| 319. | Quinoxaline-2-carboxylic acid | | 481, 464 |
| | [4(R)-carbamoyl-1(S)-(2- | | |
| | chloro-benzyl)-2(S),7- | | |
| | dihydroxy-7-methyl-octyl]- | | |
| | amide | | |
| 320. | Quinoxaline-2-carboxylic acid | 130-131 | 499 |
| | [1(S)-(2-fluoro-benzyl)-2(S),7- | | |
| | dihydroxy-4(R)- | | |
| | hydroxycarbamoyl-7-methyl- | | |
| | octyl]-amide | | |
| 321. | Quinoxaline-2-carboxylic acid | 147-148 | 483 |
| | [4(R)-carbamoyl-1(S)-(2-fluoro- | | |
| | benzyl)-2(S),7-dihydroxy-7- | | |
| | methyl-octyl]-amide | | |
| 322. | Quinoxaline-2-carboxylic acid | 150-153 | 517, 499, 466 |
| | [1(S)-(3,4-difluoro-benzyl)- | | |
| | 2(S),7-dihydroxy-4(R)- | | |
| | hydroxycarbamoyl-7-methyl- | | |
| | octyl]-amide | | |
| 323. | Quinoxaline-2-carboxylic acid | 110-120 | 501, 483, 466 |
| | [4(R)-carbamoyl-1(S)-(3,4- | | |
| | difluoro-benzyl)-2(S),7- | | |
| | dihydroxy-7-methyl-octyl]- | | |
| | amide | | |
| 324. | Quinoxaline-2-carboxylic acid | 155-158 | 515, 497, 480 |
| | (4(R)-carbamoyl-2(S),7- | | |
| | dihydroxy-7-methyl-1(S)- | | |
| | naphthalen-1-ylmethyl-octyl)- | | |
| | amide | | |

### Example 325

### Quinoxaline-2-carboxylic Acid [1-(3-fluoro-benzyl)-2,7-dihydroxy-4-(1H-imidazol-2-yl)-7-methyl-octyl]-amide

To a solution of trifluoro-acetic acid 3-(5-{2-(3-fluoro-phenyl)-1-[(quinoxaline-2-carbonyl)-amino]-ethyl}-2-oxo-tetrahydro-furan-3-yl)-1,1-dimethyl-propyl ester (212 mg, 0.378 mmol) in methanol (4 mL) was added aminoacetalaldehyde dimethyl acetal (0.375 mL, 3.44 mM) and stirred for 14 days. The reaction was concentrated to provide the crude product which was purified by silica get chromatography to yield the title compound (197 mg, 91%).

### Acetic Acid 3-(2.2-dimethoxy-ethylcarbamoyl)-142-(3-fluoro-phenyl)-1-[(quinoxaline-2-carbonyl)-amino]-ethyl-6-hydroxy-6-methyl-heptyl ester

To a solution of quinoxaline-2-carboxylic acid [4-(2,2-dimethoxyethylcarbamoyl)-1-(3-fluoro-benzyl)-2,7-dihydroxy-7-methyl-octyl]-amide (192 mg, 0.336 mmol) in pyridine (0.6 mL) was added dimethylaminopyridine (DMAP) (10 mg, 0.082 mmol) and acetic anhydride (0.093 mL, 0.984 mmol). The resulting solution was stirred for 3 hours then diluted with methylene chloride and washed with 1 M hydrochloric acid. The organic layer was dried over sodium sulfate, filtered and concentrated to give the title compound as a white foam (198 mg, 96%).

### Acetic Acid 1-{2-(3-fluoro-phenyl)-1-[(quinoxaline-2-carbonyl)-amino]-ethyl}-6-hydroxy-3-(1H-imidazol-2-yl)-6-methyl-heptyl Ester

To a solution of acetic acid 3-(2,2-dimethoxy-ethylcarbamoyl)-1-{2-(3-fluorophenyl)-1-[(quinoxaline-2-carbonyl)-amino]-ethyl}-6-hydroxy-6-methyl-heptyl ester (150 mg, 0.245 mmol) in acetic acid (2 mL) was added ammonium acetate (1.5 g 19.5 mmol). The resulting mixture was heated to 115° C. for 3 hours, cooled to ambient temperature and diluted with ethyl acetate. The solution was then neutralized with saturated aqueous sodium bicarbonate. The organic layer was dried over sodium sulfate, filtered and concentrated. Chromatography on silica gel gave the title compound (22.5 mg, 17%).

### Quinoxaline-2-carboxylic Acid [1-(3-fluoro-benzyl)-2,7-dihydroxy-4-(1H-imidazol-2-yl)-7-methvylctyl]-amide

To a solution of acetic acid 1-{2-(3-fluoro-phenyl)-1-[(quinoxaline-2-carbonyl)amino]-ethyl}-6-hydroxy-3-(1H-imidazol-2-yl)-6-methyl-heptyl ester (32 mg, 0.058 mmol) in methanol (1 mL) was added potassium carbonate (100 mg, 0.724 mmol). The resulting solution was stirred for 2 hours then concentrated. The crude product was dissolved in a mixture of methylene chloride and water. The organic layer was dried over sodium sulfate, filtered and concentrated. Chromatography on silica gel gave the title compound (32 mg, >100%).

The title compounds for examples 326-339 were prepared by a method analogous to that described in Example 325.

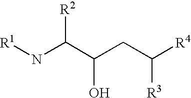

| EXAMPLE | R¹ | R² | R³ | R⁴ |
|---|---|---|---|---|
| 326 | 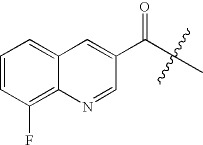 | 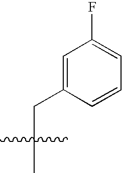 | 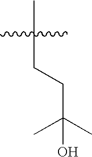 | 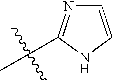 |
| 327 | 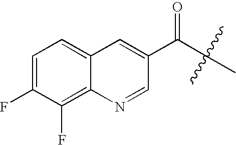 | 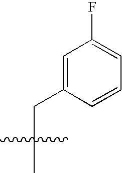 | 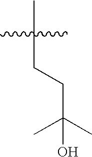 | 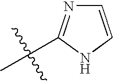 |
| 328 | 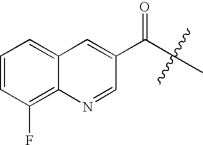 | 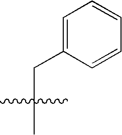 | 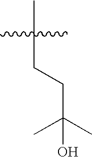 | 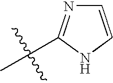 |
| 329 | 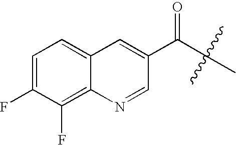 | 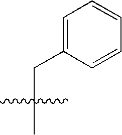 | 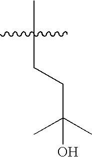 | 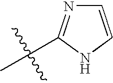 |
| 330 | 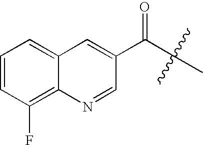 | 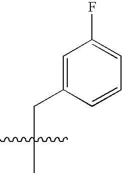 | 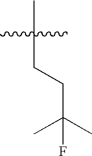 | 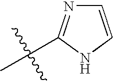 |
| 331 | 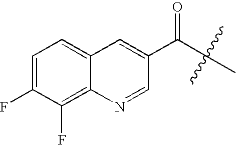 | 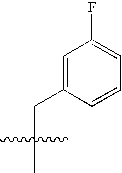 | 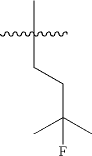 | 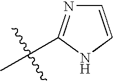 |
| 332 | 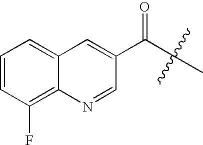 | 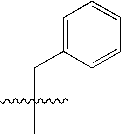 | 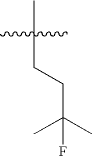 | 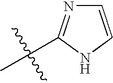 |
| 333 | 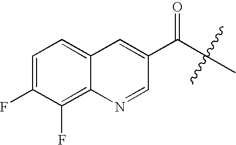 | 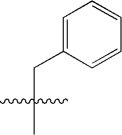 | 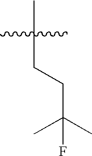 | 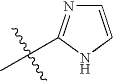 |
| 334 | 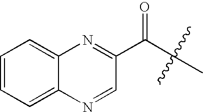 | 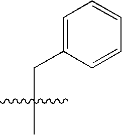 | 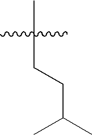 | 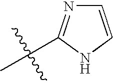 |
| 335 | 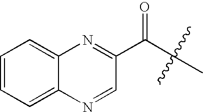 | 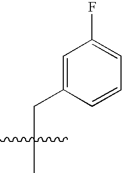 | 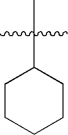 | 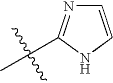 |
| 336 | 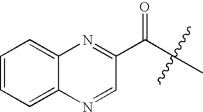 | 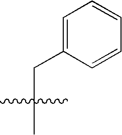 | 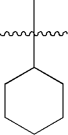 | 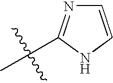 |
| 337 | 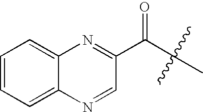 | 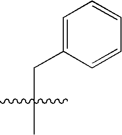 | 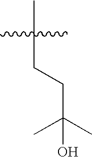 | 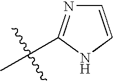 |
| 338 | 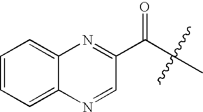 | 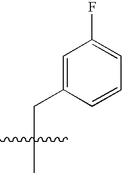 | 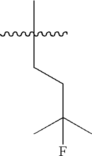 | 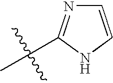 |
| 339 | 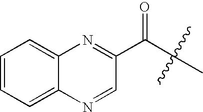 | 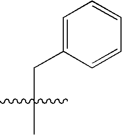 | 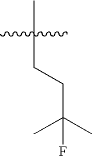 | 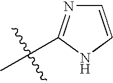 |

### Example 340

### Quinoxaline-2-carboxylic Acid [1-benzyl-7-fluoro-2-hydroxy-7-methyl-4-(4H-[1,2,4]triazol-3-yl)-octyl]-amide

### Acetic acid 3-carbamoyl-6-fluoro-6-methyl-142-phenyl-1-[(Quinoxaline-2-carbonyl)-amino]-ethyl]-heptyl ester

To a solution of quinoxaline-2-carboxylic acid (1-benzyl-4-carbamoyl-7-fluoro-2-hydroxy-7-methyl-octyl)-amide (1.01 g, 2.14 mmol) in pyridine (4 mL) was added dimethylaminopyridine (DMAP) (65 mg, 0.533 mmol) and acetic anhydride (0.400 mL, 4.23 mmol). The resulting solution was stirred for 2 hours, then diluted with methylene chloride and washed with 1 M hydrochloric acid. The organic layer was dried over sodium sulfate, filtered and concentrated to give the title compound as a white foam (1.16 g, >100%).

### Acetic acid 3-(dimethylaminomethylene-carbamoyl)-6-fluoro-6-methyl-1-{2-phenyl-1-[(quinoxaline-2-carbonyl)-amino]-ethyl}-heptyl Ester

A solution of acetic acid 3-carbamoyl-6-fluoro-6-methyl-1-(2-phenyl-1[(quinoxaline-2-carbonyl)-amino]-ethyl}-heptyl ester (522 mg, 1.03 mmol) in N,N-dimethylformamide dimethyl acetal (2 mL) was heated to 50° C. for two hours, cooled to ambient temperature and diluted with methylene chloride and water. The organic layer was washed with saturated aqueous sodium chloride, dried over sodium sulfate, filtered and concentrated to give the title compound as a white foam (580 mg, 100%).

### Acetic Acid 6-fluoro-6-methyl-142-phenyl-1-[(quinoxaline-2-carbonyl)-amino]ethyl-3-(4H-[1,2,4]triazol-3-yl)-heptyl Ester

To a solution of acetic acid 3-(dimethylaminomethylene-carbamoyl)-6-fluoro-6-methyl-1-{2-phenyl-1-[(quinoxaline-2-carbonyl)-amino]-ethyl}-heptyl ester (580 mg, 1.03 mmol) in acetic acid (2.5 mL) was added hydrazine (35 wt. % in water, 0.040 mL). The resulting solution was heated to 50° C. for 4 hours, cooled to ambient temperature, diluted with ethyl acetate, and neutralized with saturated aqueous sodium bicarbonate. The organic later was dried over sodium sulfate, filtered, and concentrated to give the title compound as a white foam (580 mg, >100%).

### Quinoxaline-2-carboxylic Acid [1-benzyl-7-fluoro-2-hydroxy-7-methyl-4-(4H-[1,2,4]triazol-3-yl)-octyl]-amide

To a solution of acetic acid 6-fluoro-6-methyl-1-{2-phenyl-1-[(quinoxaline-2-carbonyl)-amino]-ethyl}-3-(4H-[1,2,4]triazol-3-yl)-heptyl ester (575 mg, 1.08 mmol) in methanol (10 mL) was added potassium carbonate (276 mg, 2.00 mmol), stirred for 5 hours, and concentrated. The crude product was dissolved in ethyl acetate and water. The organic layer was then washed with saturated aqueous sodium chloride, dried over sodium sulfate, filtered and concentrated. Chromatography on silica gel gave the title compound (459 mg, 87%).

The title compounds for examples 341-342 were prepared by a method analogous to that described in Example 340.

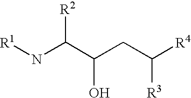

| EXAMPLE | R¹ | R² | R³ | R⁴ |
|---|---|---|---|---|
| 341 | 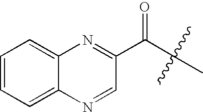 | 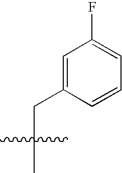 | 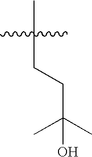 | 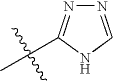 |
| 342 | 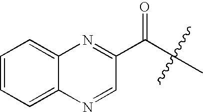 | 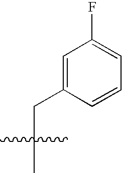 | 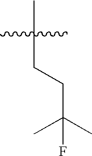 | 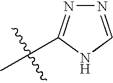 |

### Example 343

### Quinoxaline-2-carboxylic Acid [1-benzyl-4-(4,5-dihydro-1H-imidazol-2-yl)-7-fluoro-2-hydroxy-7-methyl-octyl]-amide

### Quinoxaline-2-carboxylic Acid [1-benzyl-4-(4.5-dihydro-1H-imidazol-2-yl)-7-fluoro-2-hydroxy-7-methyl-octyl]-amide

To a solution of ethylenediamine (0.040 mL, 0.598 mmol) in toluene (2 mL) at −10° C. was added trimethylaluminum (2.0 M in hexanes, 0.300 mL, 0.600 mmol) and stirred for 15 minutes. A solution of quinoxaline-2-carboxylic acid {1-[4-(3-fluoro-3-methyl-butyl)-5-oxo-tetrahydro-furan-2-yl]-2-phenyl-ethyl}-amide (250 mg, 0.556 mmol) in toluene (3 mL) was then added and the reaction warmed to ambient temperature, then heated to reflux for 3 hours. The reaction was cooled to ambient temperature and quenched carefully with water (1 mL). The solution was diluted with methylene chloride and methanol and then filtered, washing the filtrate with methanol. The organics were concentrated and the crude product was purified by chromatography on silica gel to give the title compound (74 mg, 17%).

The title compounds for examples 344-345 were prepared by a method analogous to that described in Example 343.

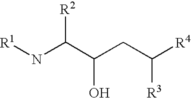

| EXAMPLE | R¹ | R² | R³ | R⁴ |
|---|---|---|---|---|
| 344 | 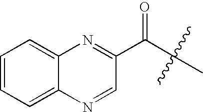 | 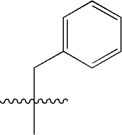 | 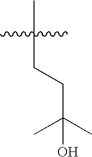 | 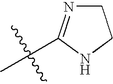 |
| 345 | 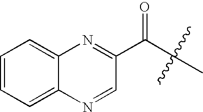 | 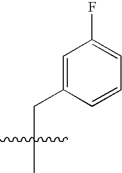 | 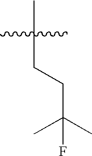 | 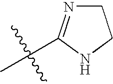 |

### Example 346

### Quinoxaline-2-carboxylic Acid [4-(5-amino-[1,3,4]oxadiazol-2-yl)-1-benzyl-7-fluoro-2-hydroxy-7-methyl-octyl]-amide

### Quinoxaline-2-carboxylic Acid (1-benzyl-7-fluoro-4-hvdrazinocarbonyl-2-hydroxy-7-methyl-octyl)-amide

To a solution of quinoxaline-2-carboxylic acid {1-[4-(3-fluoro-3-methyl-butyl)-5-oxo-tetrahydro-furan-2-yl]-2-phenyl-ethyl}-amide (220 mg, 0.489 mmol) in methanol (5 mL) was added excess hydrazine (0.500 mL) and stirred for 18 hours.

The reaction was concentrated to give the title compound (222 mg, 94%).

### Quinoxaline-2-carboxylic Acid [4-(5-amino-[1,3,4]oxadiazol-2-yl)-1-benzyl-7-fluoro-2-hydroxy-7-methyl-octyl]-amide

To a solution of quinoxaline-2-carboxylic acid (1-benzyl-7-fluoro-420 hydrazinocarbonyl-2-hydroxy-7-methyl-octyl)-amide (110 mg, 0.228 mmol) in dioxane (0.5 mL) and water (0.5 mL) was added cyanogen bromide (31 mg, 0.296 mmol) and potassium hydrogencarbonate (31 mg, 0.310 mmol). The reaction was heated to reflux for 1 hour then cooled to ambient termperature. The dioxane/water was removed by adding benzene (5 mL) and concentrating (2×). The remaining solid was dissolved in ethyl acetate and water. The layers were separated and the aqueous layer extracted with ethyl acetate. The combined organics were dried over sodium sulfate and concentrated. Recrystallization of the crude product using a mixture of ethyl acetate, hexanes and methanol gave the title compound (64 mg, 55%).

The title compounds for examples 347-357 were prepared by a method analogous to that described in Example 346.

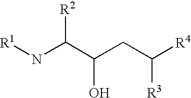

| EXAMPLE | R¹ | R² | R³ | R⁴ |
|---|---|---|---|---|
| 347 | 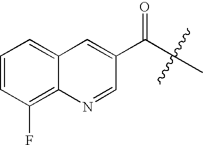 | 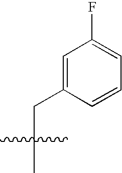 | 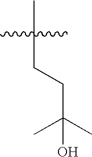 | 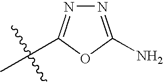 |
| 348 | 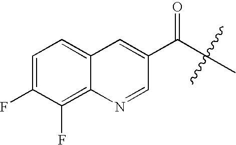 | 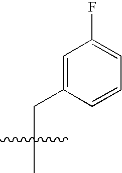 | 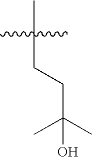 | 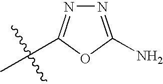 |
| 349 | 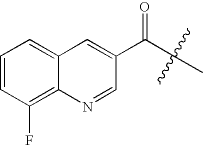 | 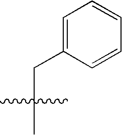 | 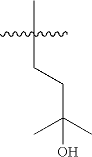 | 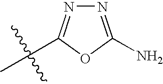 |
| 350 | 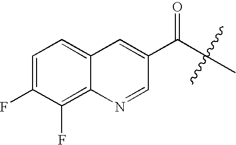 | 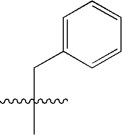 | 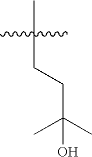 | 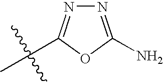 |
| 351 | 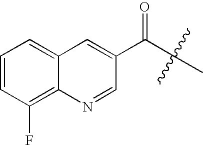 | 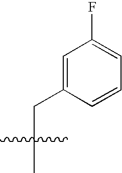 | 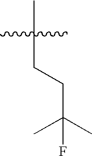 | 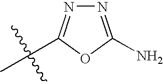 |
| 352 | 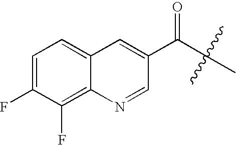 | 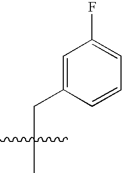 | 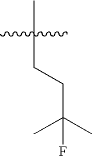 | 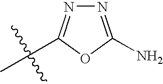 |
| 353 | 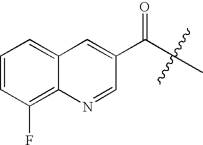 | 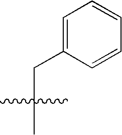 | 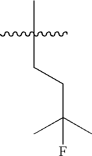 | 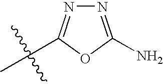 |
| 354 | 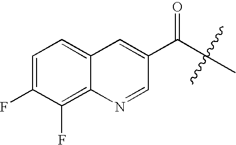 | 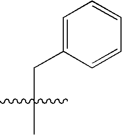 | 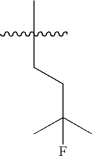 | 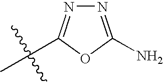 |
| 355 | 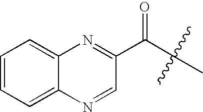 | 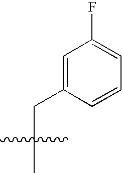 | 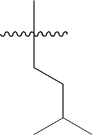 | 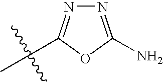 |
| 356 | 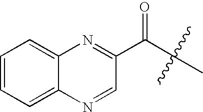 | 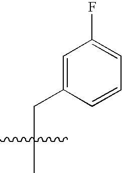 | 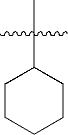 | 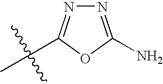 |
| 357 | 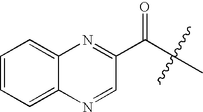 | 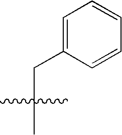 | 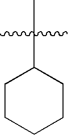 | 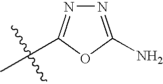 |

### Example 358

### Quinoxaline-2-carboxylic Acid [1-benzyl-7-fluoro-2-hydroxy-7-methyl-4-(5-oxo-4,5-dihydro-[1,3,4]oxadiazol-2-yl)-octyl]-amide

### Quinoxaline-2-carboxylic Acid [1-benzyl-7-fluoro-2-hydroxy-7-methyl-4-(5-oxo-4,5-dihydro-[1,3,4]oxadiazol-2-yl)-octyl]-amide

To a solution of quinoxaline-2-carboxylic acid (1-benzyl-7-fluoro-4-hydrazinocarbonyl-2-hydroxy-7-methyl-octyl)-amide (62 mg, 0.129 mmol) in tetrahydrofuran (2 mL) was added triethylamine (0.018, 0.129 mmol) at 0° C. was added carbonyldiimidazole (23 mg, 0.142 mmol). The reaction was allowed to warm to ambient temperature and stirred a total of 20 hours before diluting with ethyl acetate (10 mL) and hexane (2 mL). The mixture was washed with saturated aqueous ammonium chloride, saturated aqueous sodium bicarbonate, and saturated aqueous sodium chloride. The organic layer was dried over magnesium sulfate, filtered and concentrated. Chromatography on silica gel gave the title compound (54 mg, 82%).

The title compounds for examples 359-360 were prepared by a method analogous to that described in Example 358.

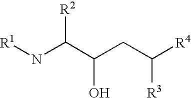

| EXAMPLE | R¹ | R² | R³ | R⁴ |
|---|---|---|---|---|
| 359 | 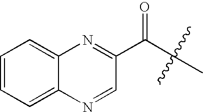 | 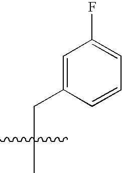 | 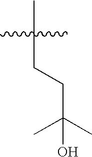 | 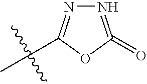 |
| 360 | 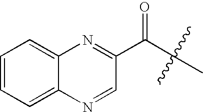 | 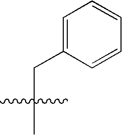 | 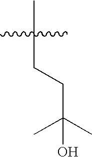 | 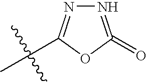 |

### Example 361

### Quinoxaline-2-carboxylic Acid [1-benzyl-4-(4.5-dihydro-oxazol-2-yl)-7-fluoro-2-hydroxy-7-methyl-octyl]-amide

### 2-(3-Fluoro-3-methyl-butyl)-4-hydroxy-6-phenyl-5-[(quinoxaline-2-carbonyl)-amino]-hexanoic Acid

To a solution of quinoxaline-2-carboxylic acid {1-[4-(3-fluoro-3-methyl-butyl)-5-oxo-tetrahydro-furan-2-yl]-2-phenyl-ethyl}-amide (4 g, 8.90 mmol) in tetrahydrofuran was added lithium hydroxide (1 M in water, 28 mL) and stirred for 2 hours. The reaction was then concentrated, and concentrated from benzene (2×) to give the title compound (4.2 g, 100%).

### 4-(tert-Butyl-dimethyl-silanyloxy)-2-(3-fluoro-3-methyl-butyl)-6-phenyl-5-[(guinoxaline-2-carbonyl)-amino]-hexanoic Acid

To a solution of 2-(3-fluoro-3-methyl-butyl)-4-hydroxy-6-phenyl-5-[(quinoxaline-2-carbonyl)-amino]-hexanoic acid (1.63 g, 3.49 mmol) in dimethylformamide (10 mL) was added t-butyldimethylsilyl choride (3.2 g, 20.9 mmol) and imidazole (2.9 g, 41.9 mmol). The reaction was stirred for 4 days then quenched with methanol and stirred another 0.5 hours. The solution was diluted with ether and water. The organic layer was washed with saturated aqueous sodium chloride, dried over magnesium sulfate, filtered and concentrated. Chromatography on silica gel gave the title compound (784 mg, 39%).

### Quinoxaline-2-carboxylic Acid [1-benzyl-2-(tert-butyl-dimethyl-silanyloxy)-7-fluoro-4-(2-hydroxy-ethylcarbamovi)-7-methylioctyl]-amide

To a solution of 4-(tert-butyl-dimethyl-silanyloxy)-2-(3-fluoro-3-methyl-butyl)-6-phenyl-5-[(quinoxaline-2-carbonyl)-amino]-hexanoic acid (515 mg, 0.885 mmol) in methylene chloride (9 mL) was added ethanolamine (0.080 mL, 1.33 mmol), 1-hydroxybenzotriazole (215 mg, 1.59 mmol), 1-(3-dimethylaminopropyl)-3-ethylcarbodiimide hydrochloride (288 mg, 1.50 mmol) and triethylamine (0.247 mL, 1.77 mmol). The resulting solution was stirred for 17 hours then diluted with ethyl acetate and washed with water then saturated aqueous sodium chloride. The organic layer was then dried over sodium sulfate, filtered, and concentrated. Chromatography on silica gel gave the title compound (343 mg, 62%).

### Quinoxaline-2-carboxylic Acid [1-benzyl-2-(tert-butyl-dimethyl-silanyloxy)-4-(4,5-dihydro-oxazol-2-yl)-7-fluoro-7-methyl-octyl-amide

To a solution of quinoxaline-2-carboxylic acid [1-benzyl-2-(tert-butyldimethyl-silanyloxy)-7-fluoro-4-(2-hydroxy-ethylcarbamoyl)-7-methyl-octyl]-amide (100 mg, 0.160 mmol) in methylene chloride (1.5 mL) was added triphenylphosphine (63 mg, 0.240 mmol), hexachloroethane (57 mg, 0.240 mmol), and triethylamine (0.045 mL, 0.320 mmol). The reaction was stirred for 2 hours than chromatographed directly on silica gel to give the title compound (72.5 mg, 75%).

### Quinoxaline-2-carboxylic Acid [1-benzyl-4-(4,5-dihydro-oxazol-2-yl)-7-fluoro-2-hydroxy-7-methyl-octyl]-amide

To a solution of quinoxaline-2-carboxylic acid [1-benzyl-2-(tert-butyl-dimethylsilanyloxy)-4-(4,5-dihydro-oxazol-2-yl)-7-fluoro-7-methyl-octyl]-amide (41 mg, 0.068 mmol) in tetrahydrofuran (0.70 mL) was added tris(dimethylamino)sulfur (trimethylsilyl)difluoride (56 mg, 0.203 mmol). The reaction was stirred for 1 hour then quenched with methanol and concentrated. Chromatography on silica gel gave the title compound (27.8 mg, 84%).

The title compounds for examples 362-373 were prepared by a method analogous to that described in Example 361.

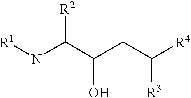

| EXAMPLE | R¹ | R² | R³ | R⁴ |
|---|---|---|---|---|
| 362 | 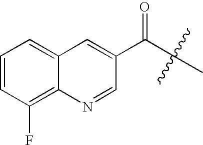 | 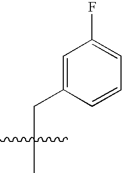 | 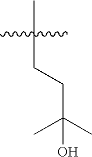 | 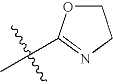 |
| 363 | 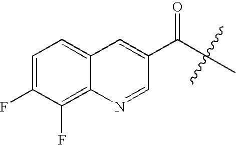 | 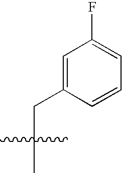 | 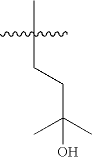 | 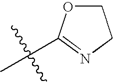 |
| 364 | 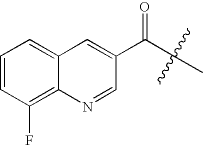 | 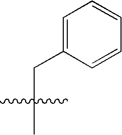 | 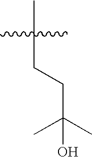 | 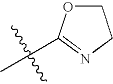 |
| 365 | 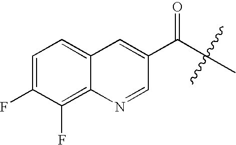 | 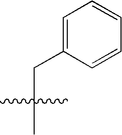 | 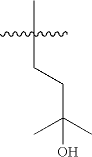 | 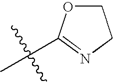 |
| 366 | 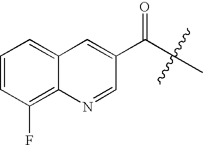 | 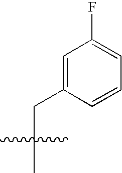 | 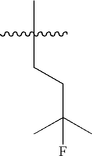 | 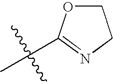 |
| 367 | 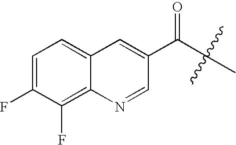 | 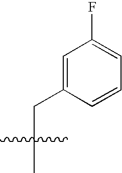 | 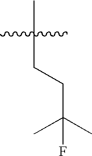 | 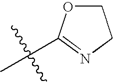 |
| 368 | 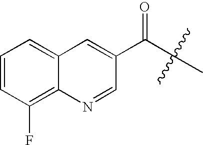 | 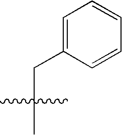 | 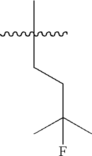 | 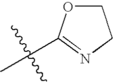 |
| 369 | 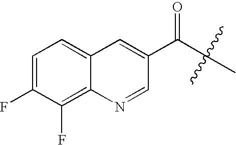 | 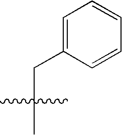 | 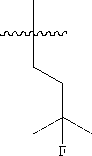 | 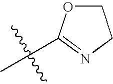 |
| 370 | 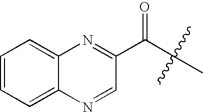 | 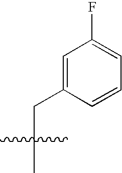 | 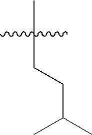 | 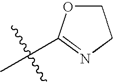 |
| 371 | 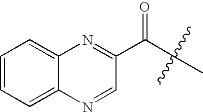 | 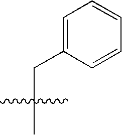 | 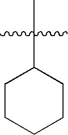 | 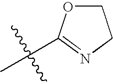 |
| 372 | 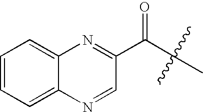 | 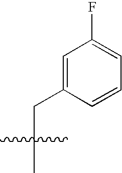 | 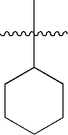 | 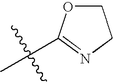 |
| 373 | 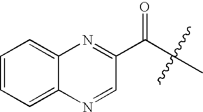 | 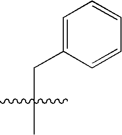 | 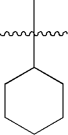 | 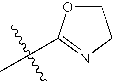 |

### Example 374

### Quinoxaline-2-carboxylic Acid (1-benzyl-7-fluoro-2-hydroxy-7-methyl-4-oxazol-2-yl-octyl)-amide

### Quinoxaline-2-carboxylic Acid [1-benzyl-2-(tert-butyl-dimethyl-silanyloxy)-7-fluoro-7-methyl-4-(2-oxo-ethylcarbamoyl)-octyl]-amide

To a solution of quinoxaline-2-carboxylic acid [1-benzyl-2-(tert-butyldimethyl-silanyloxy)-7-fluoro-4-(2-hydroxy-ethylcarbamoyl)-7-methyl-octyl]-amide (250 mg, 0.400 mmol) in methylene chloride was added 1,1,1-triacetoxy-1,1-dihydro-1,2-benziodoxol-3-(1H)-one [Dess-Martin periodinane] (340 mg, 0.800 mmol). The reaction was stirred for 2 hours and then diluted with ether and quenched with a 1:1 mixture of saturated aqueous sodium thiosulfate:sodium bicarbonate. The layers were separated and the aqueous layer extracted with ethyl acetate. The combined organics were washed with a 1:1 mixture of saturated aqueous sodium thiosulfate:sodium bicarbonate, water, and saturated sodium chloride. The organic layer was then dried over sodium sulfate, filtered, and concentrated. Chromatography on silica gel gave the title compound (233 mg, 94%).

### Quinoxaline-2-carboxylic Acid [1-benzyl-2-(tert-butyl-dimethyl-silanyloxy)-7-fluoro-7-methyl-4-oxazol-2-yl-octyl]-amide

To a solution of quinoxaline-2-carboxylic acid [1-benzyl-2-(tert-butyl-dimethyl-silanyloxy)-7-fluoro-7-methyl-4-(2-oxo-ethylcarbamoyl)-octyl]-amide (230 mg, 0.369 mmol) in methylene chloride (3.5 mL) was added triphenylphosphine (145 mg, 0.554 mmol), hexachloroethane (131 mg, 0.554 mmol) and triethylamine (0.103 mL, 0.739 mmol). The reaction was stirred for 16 hours than concentrated. Chromatography on silica gel gave the title compound (137 mg, 62%).

### Quinoxaline-2-carboxylic Acid (1-benzyl-7-fluoro-2-hydroxy-7-methyl-4-oxazol-2-yl-octyl)-amide

To a solution of quinoxaline-2-carboxylic acid [1-benzyl-2-(tert-butyl-dimethyl-silanyloxy)-7-fluoro-7-methyl-4-oxazol-2-yl-octyl]-amide (133 mg, 0.220 mmol) in tetrahydrofuran (2 mL) was added tris(dimethylamino)sulfur (trimethylsilyl)difluoride (180 mg, 0.660 mmol). The reaction was stirred for 1 hour then quenched with methanol and concentrated. Chromatography on silica gel gave the title compound (73 mg, 68%).

The title compounds for examples 375-385 were prepared by a method analogous to that described in Example 374.

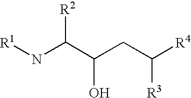

| EXAMPLE | R¹ | R² | R³ | R⁴ |
|---|---|---|---|---|
| 375 | 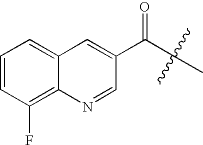 | 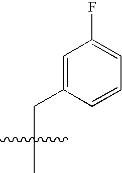 | 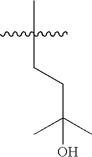 | 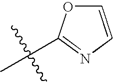 |
| 376 | 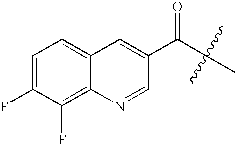 | 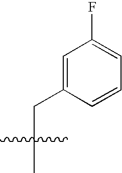 | 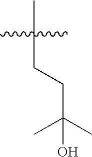 | 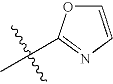 |
| 377 | 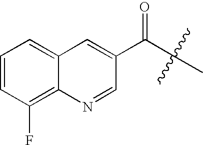 | 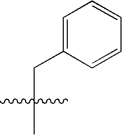 | 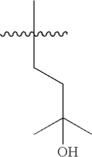 | 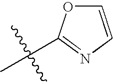 |
| 378 | 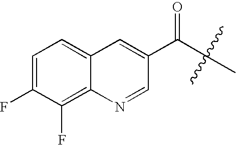 | 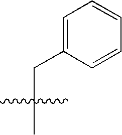 | 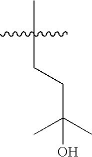 | 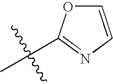 |
| 379 | 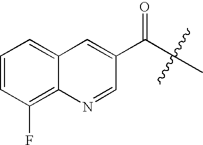 | 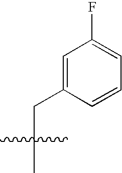 | 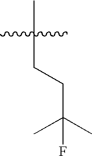 | 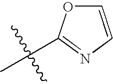 |
| 380 | 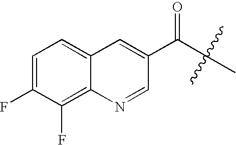 | 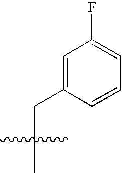 | 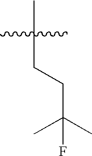 | 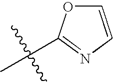 |
| 381 | 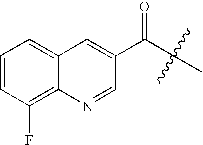 | 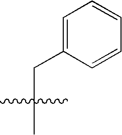 | 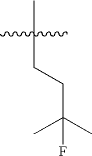 | 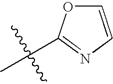 |
| 382 | 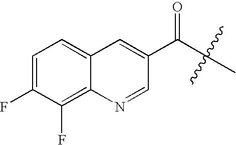 | 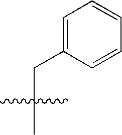 | 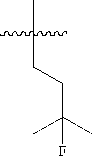 | 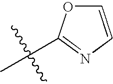 |
| 383 | 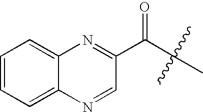 | 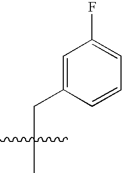 | 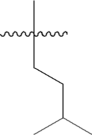 | 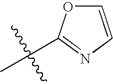 |
| 384 | 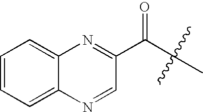 | 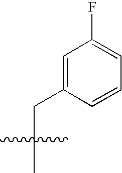 | 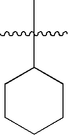 | 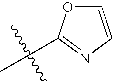 |
| 385 | 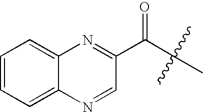 | 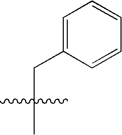 | 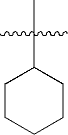 | 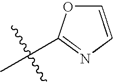 |

### Example 386

### Quinoxaline-2-carboxylic Acid (4-benzenesulfonyl-1-benzyl-2-hydroxy-7-methyl-octyl)-amide

### (4-Benzenesulfonyl-1-benzyl-2-hydroxy-7-methyl-octyl)-carbamic Acid Benzyl Ester

To a solution of 3.0 equivalents of (4-methyl-pentane-1-sulfonyl)-benzene (previously prepared by Gaoni, J. Org. Chem. 1982, 47, 2564) in tetrahydrofuran cooled to −78° C. is added 3.0 equivalents of n-butyl lithium and stirred for 30 min. One equivalent of (1-oxiranyl-2-phenyl-ethyl)-carbamic acid benzyl ester (previously prepared by Kaldor, et al. J. Med. Chem., 1997, p. 3979) in THF is then added dropwise and the reaction stirred for 1.5 h. The reaction is then quenched with saturated aqueous sodium bicarbonate and warmed to ambient temperature. After standard aqueous work-up and extraction, followed by concentration and silica gel chromatography the title compound is obtained.

### 2-Amino-5-benzenesulfonyl-8-methyl-1-phenyl-nonan-3-ol

To a solution of (4-benzenesulfonyl-1-benzyl-2-hydroxy-7-methyl-octyl)carbamic acid benzyl ester in ethanol is added 10 mole % palladium hydroxide on carbon. The mixture is then shaken on a Parr shaker under 50 psi of hydrogen for approximately 18 h. The catalyst is filtered off and the solution concentrated to give the title compound.

### Quinoxaline-2-carboxylic Acid (4-benzenesulfonyl-1-benzyl-2-hydroxy-7-methyl-octyl)-amide

To a solution of one equivalent of 2-amino-5-benzenesulfonyl-8-methyl-1 phenyl-nonan-3-ol in methylene chloride is added 1.05 equivalents each of 2-quinoxalinecarboxylic acid, N-methyl morpholine, and O-benzotriazol-1-yl-N,N,N′,N′-teteramethyluronium hexafluorophosphate. The reaction mixture is stirred at ambient temperature for 18 h. After standard aqueous work-up and extraction, followed by concentration and silica gel chromatography the title compound is obtained.

The title compounds for examples 387-396 are prepared by a method analogous to that described in Example 386.

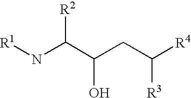

| EXAMPLE | R¹ | R² | R³ | R⁴ |
|---|---|---|---|---|
| 387 | 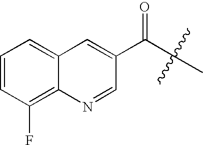 | 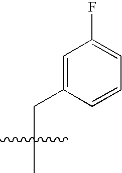 | 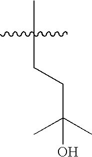 | 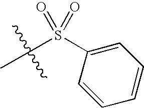 |
| 388 | 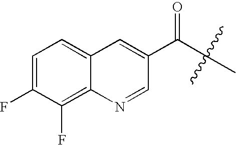 | 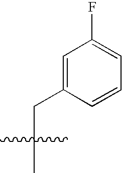 | 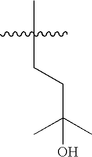 | 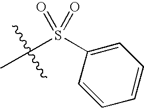 |
| 389 | 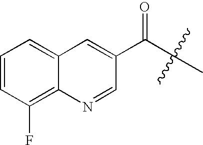 | 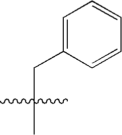 | 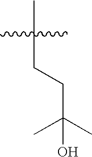 | 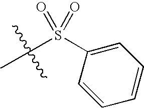 |
| 390 | 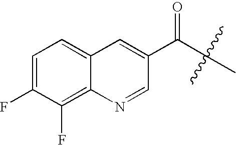 | 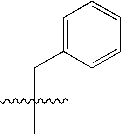 | 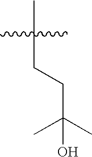 | 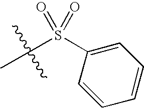 |
| 391 | 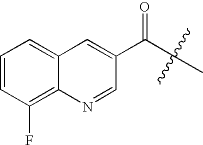 | 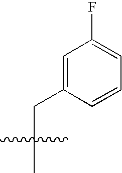 | 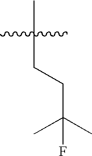 | 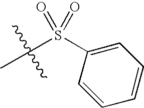 |
| 392 | 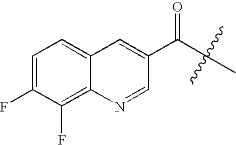 | 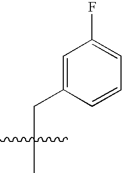 | 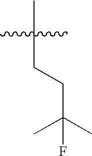 | 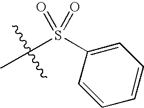 |
| 393 | 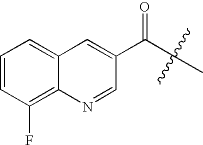 | 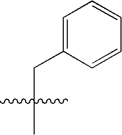 | 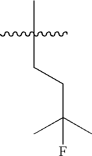 | 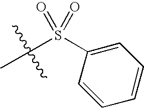 |
| 394 | 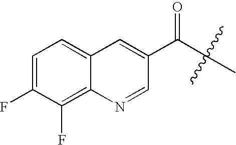 | 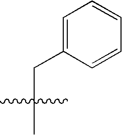 | 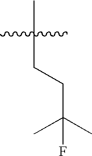 | 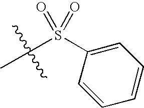 |
| 395 | 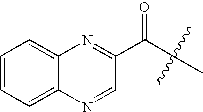 | 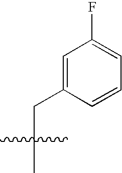 | 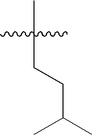 | 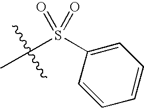 |
| 396 | 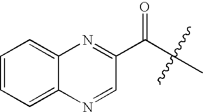 | 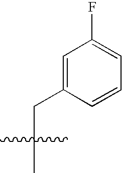 | 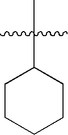 | 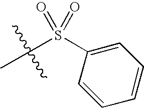 |

### Example 397

### Quinoxaline-2-carboxylic Acid (1-benzyl-7-fluoro-2-hydroxy-7-methyl-4-thiocarbamoyl-octyl)-amide

Acetic acid 6-fluoro-6-methyl-142-ghenyl-1-[(rcuinoxaline-2-carbonyl)-amino]ethyl)-3-thiocarbamoyl-heptyl Ester

To a solution of 1.0 equivalent of acetic acid 3-carbamoyl-6-fluoro-6-methyl]-{2-phenyl-1-[(quinoxaline-2-carbonyl)-amino]-ethyl}-heptyl ester in tetrahydrofuran cooled to 0° C. is added 0.5 equivalents of Lawesson's reagent dropwise. The yellow suspension is allowed to warm to room temperature and stirred for about 5 h. The reaction mixture is concentrated to dryness, then purified by silica gel chromatography to give the title compound.

### Quinoxaline-2-carboxylic Acid (1-benzyl-7-fluoro-2-hydroxy-7-methyl-4-thiocarbamoyl-octyl)-amide

To a solution of 1.0 equivalents of acetic acid 6-fluoro-6-methyl-1-{2-phenyl-1-[(quinoxaline-2-carbonyl)-amino]-ethyl}-3-thiocarbamoyl-heptyl ester in methanol is added 2.0 equivalents of potassium carbonate, stirred for approximately 5 hours, and concentrated. The crude product is dissolved in ethyl acetate and water. The organic layer is then washed with saturated aqueous sodium chloride, dried over sodium sulfate, filtered and concentrated. Chromatography on silica gel gives the title compound.

The title compounds for examples 398-400 are prepared by a method analogous to that described in Example 397.

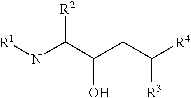

| EXAMPLE | R¹ | R² | R³ | R⁴ |
|---|---|---|---|---|
| 398 | 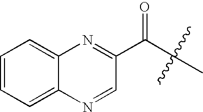 | 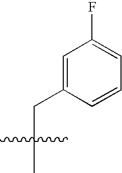 | 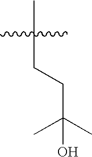 | 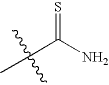 |
| 399 | 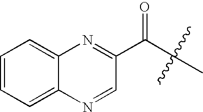 | 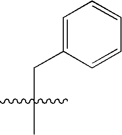 | 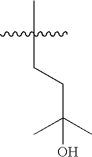 | 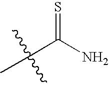 |
| 400 | 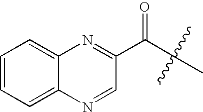 | 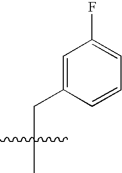 | 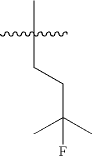 | 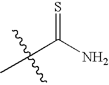 |

### Example 401

### Quinoxaline-2-carboxylic Acid (1-benzyl-4-carbamimidoyl-7-fluoro-2-hydroxy-7-methyl-octyl)-amide

### Acetic acid 3-carbamimidoyl-6-fluoro-6-methyl-1-[2-phenyl-1-[(Quinoxaline-2-carbonyl)-amino]-ethyl]-heptyl ester

To a solution of acetic acid 6-fluoro-6-methyl-1-{2-phenyl-1-[(quinoxaline-2-carbonyl)-amino]-ethyl}-3-thiocarbamoyl-heptyl ester in acetone is added excess methyl iodide. The reaction is then refluxed for approximately 2 h, then cooled and concentrated. The crude product is taken up in saturated solution of ammonia in methanol and stirred for approximately 15 hrs. The reaction mixture is concentrated to dryness, then purified by silica gel chromatography to give the title compound.

### Quinoxaline-2-carboxylic Acid (1-benzyl-4-carbamimidoyl-7-fluoro-2-hydroxy-7-methyl-octyl)-amide

To a solution of 1.0 equivalents of acetic acid 3-carbamimidoyl-6-fluoro-6-methyl-1-{2-phenyl-1-[(quinoxaline-2-carbonyl)-amino]-ethyl}-heptyl ester in methanol is added 2.0 equivalents of potassium carbonate, stirred for approximately 5 hours, and concentrated. The crude product is dissolved in ethyl acetate and water. The organic layer is then washed with saturated aqueous sodium chloride, dried over sodium sulfate, filtered and concentrated. Chromatography on silica gel gives the title compound.

The title compounds for examples 402-404 are prepared by a method analogous to that described in Example 401.

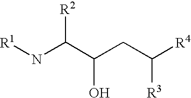

| EXAMPLE | R¹ | R² | R³ | R⁴ |
|---|---|---|---|---|
| 402 | 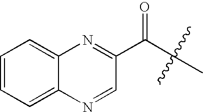 | 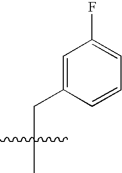 | 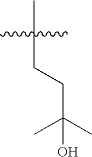 | 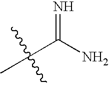 |
| 403 | 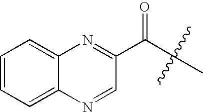 | 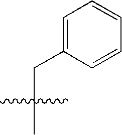 | 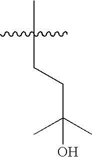 | 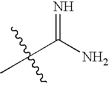 |
| 404 | 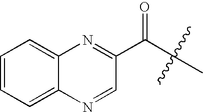 | 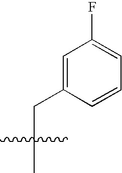 | 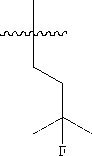 | 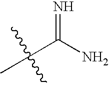 |

### Example 405

### Quinoxaline-2-carboxylic Acid [4-(acetylimino-amino-methyl)-1-benzyl-7-fluoro-2-hydroxy-7-methyl-octyl]-amide

### Qinoxaline-2-carboxylic Acid [4-(acetylimino-amino-methyl)-1-benzyl-7-fluoro-2-hydroxy-7-methyl-octyl]-amide

To a solution of 1.0 equivalents of quinoxaline-2-carboxylic acid (1-benzyl-4-carbamimidoyl-7-fluoro-2-hydroxy-7-methyl-octyl)-amide in methylene chloride is added 1.0 equivalents of triethylamine followed by 1.0 equivalents of acetyl chloride. The reaction is stirred at ambient temperature for approximately 5 hours. After standard aqueous work-up and extraction, followed by concentration and silica gel chromatography the title compound is obtained.

The title compounds for examples 406-410 are prepared by a method analogous to that described in Example 405.

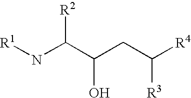

| EXAMPLE | R¹ | R² | R³ | R⁴ |
|---|---|---|---|---|
| 406 | 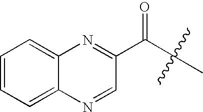 | 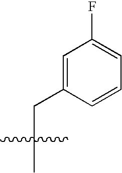 | 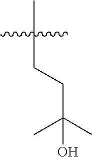 | 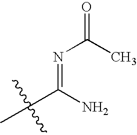 |
| 407 | 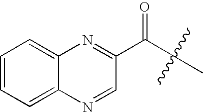 | 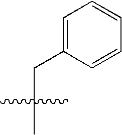 | 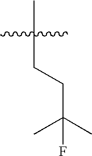 | 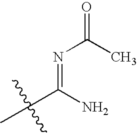 |
| 408 | 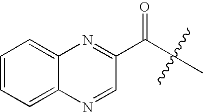 | 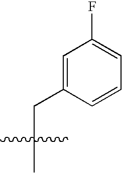 | 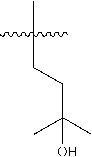 | 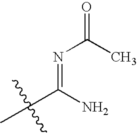 |
| 409 | 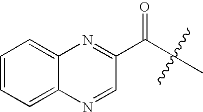 | 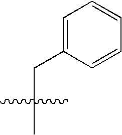 | 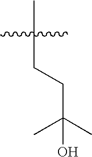 | 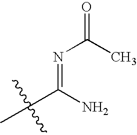 |
| 410 | 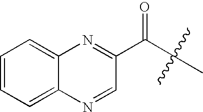 | 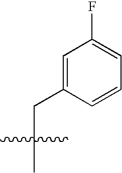 | 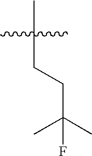 | 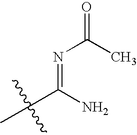 |

### Example 411

### Quinoxaline-2-carboxylic Acid [4-(amino-methanesulfonylimino-methyl)-1-benzyl-7-fluoro-2-hydroxy-7-methyl-octyl]-amide

### Quinoxaline-2-carboxylic Acid [4-(amino-methanesulfonylimino-methyl)-1-benzyl-7-fluoro-2-hydroxy-7-methyl-octyl]-amide

To a solution of 1.0 equivalents of quinoxaline-2-carboxylic acid (1-benzyl-4-carbamimidoyl-7-fluoro-2-hydroxy-7-methyl-octyl)-amide in methylene chloride is added 1.0 equivalents of triethylamine followed by 1.0 equivalents of methanesulfonyl chloride. The reaction is stirred at ambient temperature for approximately 5 hours. After standard aqueous work-up and extraction, followed by concentration and silica gel chromatography the title compound is obtained.

The title compounds for examples 412-418 are prepared by a method analogous to that described in Example 411.

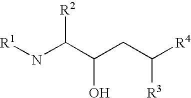

| EXAMPLE | R¹ | R² | R³ | R⁴ |
|---|---|---|---|---|
| 412 | 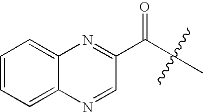 | 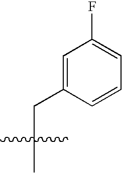 | 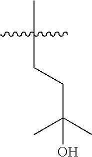 | 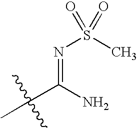 |
| 413 | 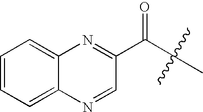 | 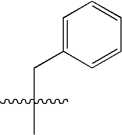 | 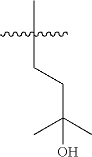 | 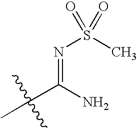 |
| 414 | 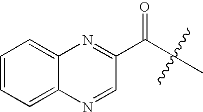 | 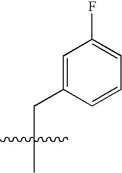 | 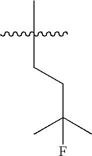 | 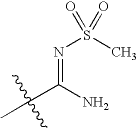 |
| 415 | 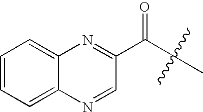 | 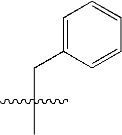 | 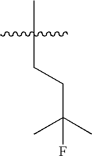 | 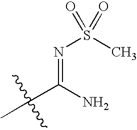 |
| 416 | 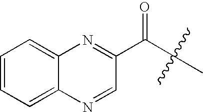 | 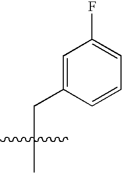 | 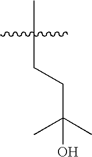 | 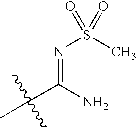 |
| 417 | 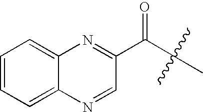 | 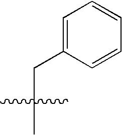 | 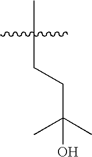 | 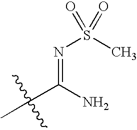 |
| 418 | 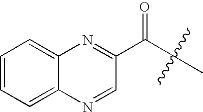 | 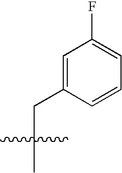 | 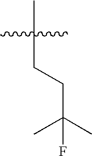 | 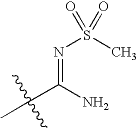 |

### Example 419

### Quinoxaline-2-carboxylic Acid [4-(cyanoimino-amino-methyl)-1-benzyl-7-fluoro-2-hydroxy-7-methyl-octyl]-amide

Quinoxaline-2-carboxylic Acid [4-(cyanoimino-amino-methyl)-1-benzyl-7-fluoro-2-hydroxy-7-methyl-octyl]-amide

To a solution of 1.0 equivalents of quinoxaline-2-carboxylic acid (1-benzyl-4-carbamimidoyl-7-fluoro-2-hydroxy-7-methyl-octyl)-amide in methylene chloride is added 1.0 equivalents of cyanogen bromide. The reaction is stirred at ambient temperature for approximately 15 hours. After standard aqueous work-up and extraction, followed by concentration and silica gel chromatography the title compound is obtained.

The title compounds for examples 420-422 are prepared by a method analogous to that described in Example 419.

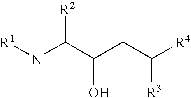

| EXAMPLE | R¹ | R² | R³ | R⁴ |
|---|---|---|---|---|
| 420 | 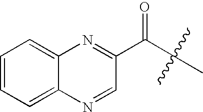 | 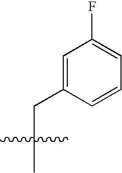 | 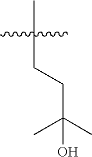 | 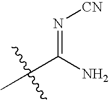 |
| 421 | 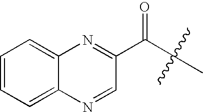 | 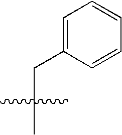 | 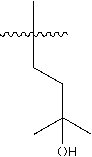 | 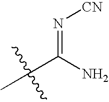 |
| 422 | 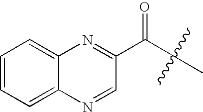 | 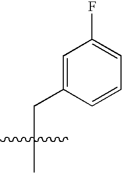 | 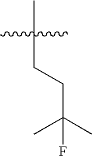 | 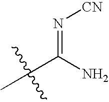 |

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating or preventing a disorder or condition selected from the group consisting of fibrosis, Alzheimer's disease, conditions associated with leptin production, sequelae associated with cancer, cancer metastasis, diseases or conditions related to production of cytokines at inflammatory sites, and tissue damage caused by inflammation induced by infectious agents; wherein the method comprises administering to a mammal in need of such treatment or prevention a pharmaceutically effective amount of a compound of formula (I) 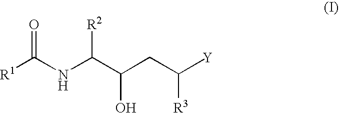
wherein R¹ is (C₂-C₉)heteroaryl optionally substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—N H—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
R² is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein m is zero, one, two, three or four; wherein each of said phenyl, naphthyl, (C₃-C₁₀)cycloalkyl and (C₂-C₉)heteroaryl moieties of said phenyl-(CH₂)ₘ—, naphthyl-(CH₂)ₘ—, (C₃-C₁₀)cycloalkyl-(CH₂)ₘ— and (C₂-C₉)heteroaryl(CH₂)ₘ— groups may optionally be substituted with one or more substituents, wherein each substituent is independently hydrogen, halo, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl-(C═O)O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N(C═O)—, H₂N(C═O)—(C₁-C₆)alkyl, (C₁-C₆)alkyl-HN(C═O)—(C₁-C₆)alkyl, [(C₁-C₆)alkyl]₂N(C═O)—(C₁-C₆)alkyl, H(O═C)—NH—, (C₁-C₆)alkyl(C═O)—NH, (C₁-C₆)alkyl(C═O)[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, or (C₂-C₉)heteroaryl;
R³ is hydrogen, (C₁-C₁₀)alkyl, (C₃-C₁₀)cycloalkyl-(CH₂)ₙ—, (C₂Cg)heterocycloalkyl-(CH₂)ₙ—, (C₂-C₉)heteroaryl-(CH₂)ₙ— or aryl-(CH₂)ₙ—; wherein n is zero, one, two, three, four, five or six;
wherein the (C₁-C₁₀)alkyl moiety of said R³ (C₁-C₁₀)alkyl group may optionally be substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—N H, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and wherein any of the carbon-carbon single bonds of said (C₁-C₁₀)alkyl may optionally be replaced by a carbon-carbon double bond;
wherein the (C₃-C₁₀)cycloalkyl moiety of said R³ (C₃-C₁₀)cycloalkyl-(CH₂)ₙ— group may optionally be substituted by one to three substitutents, wherein each substituent is independently hydrogen, halo, CN, (C₁-C₆)alkyl, hydroxy, hydroxy(C₁-C₆)alkyl, (C₁-C₆)alkoxy, (C₁-C₆)alkoxy(C₁-C₆)alkyl, HO—(C═O)—, (C₁-C₆)alkyl-O(C═O)—, HO—(C═O)—(C₁-C₆)alkyl, (C₁-C₆)alkyl-O—(C═O)—(C₁-C₆)alkyl, (C₁-C₆)alkyl(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)-[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂NSO₂—, H₂N—SO₂—(C₁-C₆)alkyl, (C₁-C₆)alkylHN—SO₂—(C₁-C₆)alkyl, [(C₁-C₆)alkyl]₂N—SO₂(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
wherein the (C₂-C₉)heterocycloalkyl moiety of said R³ (C₂-C₉)heterocycloalkyl(CH₂)ₙ— group comprises nitrogen, sulfur, oxygen, >S(═O), >SO₂ or >NR⁶, wherein said (C₂-C₉)heterocycloalkyl moiety of said (C₂-C₉)heterocycloalkyl-(CH₂)ₙ— group may optionally be substituted on any of the ring carbon atoms capable of forming an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
wherein the (C₂-C₉)heteroaryl moiety of said R³ (C₂-C₉)heteroaryl-(CH₂)ₙ— group comprises nitrogen, sulfur or oxygen wherein said (C₂-C₉)heteroaryl moiety of said (C₂-C₉)heteroaryl-(CH₂)ₙ— group may optionally be substituted on any of the ring carbon atoms capable of forming an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—N H—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and
wherein said aryl moiety of said R³ aryl-(CH₂)ₙ— group is optionally substituted phenyl or naphthyl, wherein said phenyl and naphthyl may optionally be substituted with from one to three substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O(C₁-C₆)alkyl, H(O═C)—, H(O═C)—(C₁-C₆)alkyl, (C₁-C₆)alkyl(O═C)—, (C₁-C₆)alkyl(O═C)(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
or R³ and the carbon to which it is attached form a five to seven membered carbocyclic ring, wherein any of the carbon atoms of said five membered carbocyclic ring may optionally be substituted with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O(C₁-C₆)alkyl, H(O═C)—, H(O═C)—(C₁-C₆)alkyl, (C₁-C₆)alkyl(O═C)—, (C₁-C₆)alkyl(O═C)(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl)₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; wherein one of the carbon-carbon bonds of said five to seven membered carbocyclic ring may optionally be fused to an optionally substituted phenyl ring, wherein said phenyl substitutents may be hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—N H—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
Y is (C₂-C₉)heteroaryl, (C₂-C₉)heterocycloalkyl, R⁵(R)⁶N-sulfonyl or a group of the formula 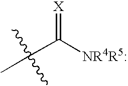
X is O, S, or NR¹²;
R⁴ is hydrogen, (C₁-C₆)alkyl, hydroxy, (C₁-C₆)alkoxy, hydroxy(C₁-C₆)alkyl, (C₁-C₆)alkoxy(C═O)—, (C₃-C₁₀)cycloalkyl-(CH₂)ₚ—, (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ—, wherein p is zero, one, two, three or four; wherein said (C₂-C₉)heterocycloalkyl, (C₂-C₉)heteroaryl, phenyl and naphthyl groups of said (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ— may be optionally substituted on any of the ring atoms capable of supporting an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C), (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino (C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[NH](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
or R⁴ and R⁵ together with the nitrogen atom to which they are attached form a (C₂-C₉)heterocycloalkyl group wherein any of the ring atoms of said (C₂-C₉)heterocycloalkyl group may optionally be substituted with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C), (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino (C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)-[N H](C₁-C₆)alkyl, (C₁-C₆)alkyl(C═O)[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
R⁵ is hydrogen, (C₁-C₆)alkyl or amino;
R⁶ is hydrogen, (C₁-C₆)alkyl, (C₁-C₆)alkoxy-(CH₂)_{g}—, (C₁-C₆)alkoxy(C═O)(CH₂)_{g}—, (C₁-C₆)alkyl-(SO₂)—(CH₂)_{g}—, (C₆-C₁₀)aryloxy-(CH₂)_{g}—, (C₆-C₁₀)aryloxy(C═O)(CH₂)_{g}—, or (C₆-C₁₀)aryl-(SO₂)—(CH₂)_{g}—, wherein g is an integer from zero to four; and
R¹² is hydrogen, CN, (C═O)-(C₁-C₉)alkyl, or (SO₂)-(C₁-C₉)alkyl;
with the proviso that when either R⁴ or R⁵ is hydrogen, and the other of R⁴ or R⁵ is (C₁-C₆)alkyl, R² is (C₃-C₁₀)cycloalkyl or isopropyl and R³ is (C₃-C₅)alkyl, phenyl, methylvinyl, dimethylvinyl, halovinyl, hydroxy(C₁-C₃)alkyl or amino(C₁-C₄)alkyl then R¹ must be other than indol-5-yl, 6-azaindol-2-yl, 2,3-dichloro-pyrol-5-yl, 4-hydroxyquinolin-3-yl, 2-hydroxyquinoxalin-3-yl, 6-azaindolin-3-yl, or optionally substituted indol-2 or 3-yl;
or a pharmaceutically acceptable form thereof.

2. The method according to claim 1, wherein said compound of formula I has the formula Ia 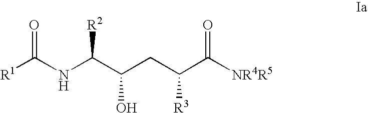
wherein R¹, R², R³, R⁴ and R⁵ are as described in claim 1.

3. The method according to claim 2, wherein R¹ is optionally substituted pyrazolo[3,4-b]pyridinyl, cinnolinyl, pyridinyl, 6,7-dihydro-5H-[1]pyrindinyl, benzothiazolyl, indolyl, pyrazinyl, benzoimidazolyl, benzofuranyl, benzo[b]thiophenyl, naphthalenyl, quinoxalinyl, isoquinolinyl, 5,6,7,8-tetrahydro-quinolin-3-yl or quinolinyl.

4. The method according to claim 2, wherein R¹ is optionally substituted pyrazolo[3,4-b]pyridin-5-yl, cinnolin-4-yl, pyridin-2-yl, 6,7-dihydro-5H-[1]pyrindin-3-yl, benzothiazol-2-yl, indol-2-yl, pyrazin-2-yl, benzoimidazol-2-yl, benzofuran-2-yl, benzo[b]thiophen-2-yl, naphthalen-2-yl, quinoxalin-2-yl, quinoxalin-6-yl, isoquinolin-1-yl, isoquinolin-3-yl, isoquinolin-4-yl, 5,6,7,8-tetrahydro-quinolin-3-yl, quinolin-2-yl, quinolin-3-yl, quinolin-4-yl or quinolin-6-yl.

5. The method according to claim 2, wherein R¹ is optionally substituted quinoxalin-2-yl, quinoxalin-6-yl, quinolin-2-yl, quinolin-3-yl, quinolin-4-yl or quinolin-6-yl.

6. The method according to claim 2, wherein R² is optionally substituted benzyl.

7. The method according to claim 2, wherein R³ is optionally substituted (C₁-C₁₀)alkyl or (C₃-C₁₀)cycloalkyl-(CH₂)ₙ—.

8. The method according to claim 2, wherein R³ is optionally substituted n-butyl, t-butyl, isobutyl, n-pentyl, 2-methyl-pentyl, cyclopentyl, or cyclohexyl.

9. The method according to claim 2, wherein R³ is substituted by fluoro or hydroxy.

10. The method according to claim 2, wherein R³ is 4,4-difluoro-cyclohexylmethyl, 2-fluoro-2-methyl-butyl, isobutyl, or 1-hydroxy-cyclohexyl.

11. The method according to claim 2, wherein the compound is:
quinoxaline-2-carboxylic acid 4(R)-carbamoyl-1(S)-(3-chloro-benzyl)-2(S),7-dihydroxy-7-methyl-octyl]-amide;
7,8-difluoro-quinoline-3-carboxylic acid (1S)-benzyl-4(R)-carbamoyl-2(S),7-dihydroxy-7-methyl-octyl)-amide;
6,7,8-trifluoro-quinoline-3-carboxylic acid (1 (S)-benzyl-4(R)-carbamoyl-2(S),7-dihydroxy-7-methyl-octyl)-amide;
quinoxaline-2-carboxylic acid [4(R)-carbamoyl-1 (S)-(3-fluoro-benzyl)-2(S),7-dihydroxy-7-methyl-octyl]-amide;
quinoxaline-2-carboxylic acid (1 (S)-benzyl-2(S),7-dihydroxy-4(R)-hydroxycarbamoyl-7-methyl-octyl)-amide;
quinoxaline-2-carboxylic acid [4(R)-carbamoyl-1 (S)-(2-chloro-benzyl)-2(S),7-dihydroxy-7-methyl-octyl]-amide;
quinoxaline-2-carboxylic acid [1 (S)-(2-fluoro-benzyl)-2(S),7-dihydroxy-4(R)-hydroxycarbamoyl-7-methyl-octyl]-amide;
quinoxaline-2-carboxylic acid [4(R)-carbamoyl-1 (S)-(2-fluoro-benzyl)-2(S),7-dihydroxy-7-methyl-octyl]-amide;
quinoxaline-2-carboxylic acid [1 (S)-(3,4-difluoro-benzyl)-2(S),7-dihydroxy-4(R)-hydroxycarbamoyl-7-methyl-octyl]-amide;
quinoxaline-2-carboxylic acid [4(R)-carbamoyl-1 (S)-(3,4-difluoro-benzyl)-2(S),7-dihydroxy-7-methyl-octyl]-amide; or
quinoxaline-2-carboxylic acid (4(R)-carbamoyl-2(S),7-dihydroxy-7-methyl-1(S)-naphthalen-1-ylmethyl-octyl)-amide.

12. The method according to claim 1, wherein the compound is administered as a 15 composition comprising the compound of formula I or Ia and a pharmaceutically acceptable carrier.

13. The method according to claim 12, wherein the disorder or condition is selected from the group consisting of pulmonary fibrosis, fibrosis associated with end-stage renal disease, fibrosis caused by radiation, tubulointerstitial fibrosis, subepithelial fibrosis, scleroderma, hepatic fibrosis, primary and secondary biliary cirrhosis, obesity, cachexia, anorexia, type II diabetes, hyperlipidemia and hypergonadism, sequelae associated with multiple myeloma, breast cancer, joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith, viral induced encephalomyelitis or demyelination, gastrointestinal inflammation, bacterial meningitis, cytomegalovirus, adenoviruses, Herpes viruses, fungal meningitis, lyme disease, and malaria.
